(19) United States
(12) Patent Application Publication
(10) Pub. No.: US 2024/0398853 A1
(43) Pub. Date: Dec. 5, 2024

(54) MULTIVALENT POLYCATION INHIBITION OF POLYANIONS IN BLOOD

(71) Applicants: The Regents of the University of Michigan, Ann Arbor, MI (US); The University of British Columbia, Vancouver (CA)

(72) Inventors: James H. Morrissey, Ann Arbor, MI (US); Jayachandran N. Kizhakkedathu, New Westminster (CA); Chanel C. La, Vancouver (CA); Charles A. Hayes, Vancouver (CA); Stephanie A. Smith, Ann Arbor, MI (US); Sreeparna Vappala, Vancouver (CA)

(21) Appl. No.: 18/695,064

(22) PCT Filed: Sep. 21, 2022
(86) PCT No.: PCT/US22/44259

§ 371 Date: Mar. 25, 2024

Related U.S. Application Data
(60) Provisional application No. 63/247,635, filed on Sep. 23, 2021.

(51) Int. Cl. A61K 31/785; A61K 47/60; A61P 7/04

(52) U.S. Cl. CPC A61K 31/785; A61K 47/60; A61P 7/04

(57) ABSTRACT

Provided herein are compositions and methods to prevent and to treat thrombosis, to reverse the anticoagulant action of heparin, and to prevent and to treat the thrombotic and antifibrinolytic effect of nucleic acids. In particular, provided herein are compositions, methods, kits, systems and uses for switchable charge state multivalent biocompatible polycations for polyanion inhibition in blood.

This invention was made with government support under HL120877 and HL135823 awarded by the National Institutes of Health. The government has certain rights in the invention.

### FIELD

Provided herein are compositions and methods to prevent and to treat thrombosis, to reverse the anticoagulant action of heparin, and to prevent and to treat the thrombotic and antifibrinolytic effect of nucleic acids. In particular, provided herein are compositions, methods, kits, systems and uses for switchable charge state multivalent biocompatible polycations for polyanion inhibition in blood.

### BACKGROUND

Synthetic polycations provide biological applications as binding partners for polyanions including, for example, polyphosphates, heparin, and extracellular nucleic acids, due to strong affinity for their polyanion ligands. However, polycations are notorious for nonspecific interactions with negatively charged components in the blood and body with consequential deleterious off-target effects. An unmet challenge to the present is to increase the binding affinity and selectivity of biologic and clinical polycations while enhancing their biocompatibility.

Synthetic polycations provide applications in a diversity of biological settings, including gene delivery via interaction with the phosphate backbone of DNA (Liu, Z. et al. Hydrophobic Modifications of Cationic Polymers for Gene Delivery. Prog. Polym. Sci. 2010, 35 (9), 1144-1162., Tseng, W.-C. et al. The Role of Dextran Conjugation in Transfection Mediated by Dextran-Grafted Polyethylenimine. J. Gene Med. 2004, 6 (8), 895-905., and Pandey, A. P. and Sawant, K. K. Polyethylenimine: A Versatile, Multifunctional Non-Viral Vector for Nucleic Acid Delivery. Mater. Sci. Eng. C 2016, 68, 904-918.), lipid design in the delivery of nucleic acid medicines (Semple, S. C. et al. Rational Design of Cationic Lipids for SiRNA Delivery. Nat. Biotechnol. 2010, 28 (2), 172-176., Allen, T. M. and Cullis, P. R. Liposomal Drug Delivery Systems: From Concept to Clinical Applications. Adv. Drug Deliv. Rev. 2013, 65 (1), 36-48.), drug delivery vehicles (Kalaska, B. et al. Heparin-Binding Copolymer Reverses Effects of Unfractionated Heparin, Enoxaparin, and Fondaparinux in Rats and Mice. Transl. Res. 2016, 177, 98-112., Kim, K. et al. Polycations and Their Biomedical Applications. Prog. Polym. Sci. 2016, 60, 18-50., and Cole, H. et al. Nanoparticle Antigen Uptake in Epithelial Monolayers Can Predict Mucosal but Not Systemic in Vivo Immune Response by Oral Delivery. Carbohydr. Polym. 2018, 190, 248-254., Tarasova, E. et al. Cytocompatibility and Uptake of Polycations-Modified Halloysite Clay Nanotubes. Appl. Clay Sci. 2019, 169, 21-30.), anti-inflammatory agents (Lee, J. et al. Nucleic Acid-Binding Polymers as Anti-Inflammatory Agents. Proc. Natl. Acad. Sci. 2011, 108 (34), 14055-14060., and Gunasekaran, Pet al. Cationic Amphipathic Triazines with Potent Anti-Bacterial, Anti-Inflammatory and Anti-Atopic Dermatitis Properties. Sci. Rep. 2019, 9 (1), 1292.) and immunomodulatory applications (Yim, H. et al. A Self-Assembled Polymeric Micellar Immunomodulator for Cancer Treatment Based on Cationic Amphiphilic Polymers. Biomaterials 2014, 35 (37), 9912-9919., and Wusiman, A. et al., Macrophage Immunomodulatory Activity of the Cationic Polymer Modified PLGA Nanoparticles Encapsulating Alhagi Honey Polysaccharide. Int. J. Biol. Macromol. 2019, 134, 730-739.). Synthetic polycations may serve as binding partners for polyanions (for example, polyphosphates, heparins, nucleic acids), and the binding behavior can be tuned for specificity with the ability to control molecular weight, geometry, and charge distribution, thereby providing attenuation of nonspecific interactions. (La, C. et al. Targeting Biological Polyanions in Blood: Strategies toward the Design of Therapeutics. Biomacromolecules 2020, 21 (7), 2595-2621.) Polycations tested for these applications include polyethyleneimine (PEI) (Liu, ibid and Pandey, ibid) poly-L-lysine (PLL) (Zauner, W. et al. Polylysine-Based Transfection Systems Utilizing Receptor-Mediated Delivery. Adv. Drug Deliv. Rev. 1998, 30 (1), 97-113.), polyallylamine (Boussif, O. et al. Synthesis of Polyallylamine Derivatives and Their Use as Gene Transfer Vectors in Vitro. Bioconjug. Chem. 1999, 10 (5), 877-883.), and polyamidoamine (PAMAM) dendrimers (Caminade, A.-M. and Majoral, J.-P. Which Dendrimer to Attain the Desired Properties? Focus on Phosphorhydrazone Dendrimers. Molecules 2018, 23 (3), 622.). Though polycations have demonstrated utility in certain settings (Godbey, W. T. et al. Poly(Ethylenimine) and Its Role in Gene Delivery. J. Controlled Release 1999, 60 (2), 149-160., Kunath, K. et al. Low-Molecular-Weight Polyethylenimine as a Non-Viral Vector for DNA Delivery: Comparison of Physicochemical Properties, Transfection Efficiency and in Vivo Distribution with High-Molecular-Weight Polyethylenimine. J. Controlled Release 2003, 89 (1), 113-125., and Lungwitz, U et al. Polyethylenimine-Based Non-Viral Gene Delivery Systems. Eur. J. Pharm. Biopharm. 2005, 60 (2), 247-266.), their highly charged state causes toxicity due to nonspecific binding to cell surfaces and circulating proteins, as well as delayed toxicity from complexing with DNA and associating with cellular processing. (Godbey, W. T, et al. Improved Packing of Poly(Ethylenimine)/DNA Complexes Increases Transfection Efficiency. Gene Ther. 1999, 6 (8), 1380-1388.) This dilemma exemplifies the conventional balance sought in the design of a polycationic therapeutic system. While the cationic charges are necessary for the targeted site and application, a preponderance of biological surfaces are polyanionic. (Bernkop-Schn0rch, A. Strategies to Overcome the Polycation Dilemma in Drug Delivery. Adv. Drug Deliv. Rev. 2018, 136-137, 62-72.). When administered into the circulatory system, nonspecific binding events result in formation of harmful clusters of cells including blood cells and other cell surfaces, and negatively charged plasma proteins, which results in toxicity. (Fischer, D. et al. A Novel Non-Viral Vector for DNA Delivery Based on Low Molecular Weight, Branched Polyethylenimine: Effect of Molecular Weight on Transfection Efficiency and Cytotoxicity. Pharm. Res. 1999, 16 (8), 1273-1279.) Factors that contribute to the toxicity of polycations include molecular weight, degree of branching, strength of charge, ionic strength in solution, zeta potential, and particle size. (Kircheis, R. et al. Polycation-Based DNA Complexes for Tumor-Targeted Gene Delivery in Vivo. J. Gene Med. 1999, 1 (2), 111-120., Alazzo, A. et al. Investigating the Intracellular Effects of Hyperbranched Polycation-DNA Complexes on Lung Cancer Cells Using LC-MS-Based Metabolite Profiling. Mol. Omics 2019, 15 (1), 77-87., and Hu, Y. et al. Kinetic Control in Assembly of Plasmid DNA/Polycation Complex Nanoparticles. ACS Nano 2019, 13 (9), 10161-10178.).

Attempts to increase the biocompatibility of polycations have been reported. (Kim, Y. et al. Polyethylenimine with Acid-Labile Linkages as a Biodegradable Gene Carrier. J. Controlled Release 2005, 103 (1), 209-219., Hu, J. et al., A Biodegradable Polyethylenimine-Based Vector Modified by Trifunctional Peptide R18 for Enhancing Gene Transfection Efficiency In Vivo. PLOS ONE 2016, 11 (12), e0166673., and Wang, W. et al. Engineering Biodegradable Micelles of Polyethylenimine-Based Amphiphilic Block Copolymers for Efficient DNA and SiRNA Delivery. J. Controlled Release 2016, 242, 71-79.). Further examples include generation of biodegradable polycations and charge shielding utilizing surface functionalization with certain functional groups or neutral polymers such as polyethylene glycol or polyglycerol. (Hellmund, M. et al. Adjustment of Charge Densities and Size of Polyglycerol Amines Reduces Cytotoxic Effects and Enhances Cellular Uptake. Biomater. Sci. 2015, 3 (11), 1459-1465., and Ghasemi, A. eta al. Synthesis and Characterization of Polyglycerol Coated Superparamagnetic Iron Oxide Nanoparticles and Cytotoxicity Evaluation on Normal Human Cell Lines. Colloids Surf Physicochem. Eng. Asp. 2018, 551, 128-136.). Whereas these methods decrease toxicity of polycations either by decreasing the charge density or by reducing the molecular size upon biodegradation, they have major limitations due to shrouding of charges resulting in decreased binding to the target site when polycations are surface modified with neutral polymers. In conventional biodegradable systems, breakdown of the polymer backbone causes decreased binding to target site after degradation. Hence, it is crucial to develop polycations that are stable in physiological conditions while providing improved biocompatibility and binding capacity. Other than shrouding the cationic charge, another approach has been to improve the biocompatibility of synthetic polycations using polymeric systems or nanosystems that differ in charge state with pH of the medium. (Thambi, T. et al. Stimuli-Sensitive Injectable Hydrogels Based on Polysaccharides and Their Biomedical Applications. Macromol. Rapid Commun. 2016.). pH-sensitive polymers typically contain simple functional groups such as amines or carboxylic acids that can be protonated or deprotonated. (Bazban-Shotorbani, S. et al. Revisiting Structure-Property Relationship of PH-Responsive Polymers for Drug Delivery Applications. J. Controlled Release 2017, 253, 46-63., and Wei, R. et al. Bidirectionally PH-Responsive Zwitterionic Polymer Hydrogels with Switchable Selective Adsorption Capacities for Anionic and Cationic Dyes. Ind. Eng. Chem. Res. 2018, 57 (24), 8209-8219.). For example, polymers composed of monomers with pendant primary, secondary, and tertiary amines adopt a protonated state at low pH. The changed state of charge is a direct result of the pendant amines accepting protons when the pH of the environment is lower than their respective pKₐ values, and releasing the protons when the polymers are moved to environments with pH higher than the pKₐ.

Synthetic polycations have been developed as targeted inhibitors of polyphosphates (polyP). (Smith, S. A. et al. Inhibition of Polyphosphate as a Novel Strategy for Preventing Thrombosis and Inflammation. Blood 2012, 120 (26), 5103-5110., Shenoi, R. A. et al. Affinity-Based Design of a Synthetic Universal Reversal Agent for Heparin Anticoagulants. Sci. Transl. Med. 2014, 6 (260), 260ra150., Travers, R. J. et al. Nontoxic Polyphosphate Inhibitors Reduce Thrombosis While Sparing Hemostasis. Blood 2014, 124 (22), 3183-3190., Kalathottukaren, M. et al. Alteration of Blood Clotting and Lung Damage by Protamine Are Avoided Using the Heparin and Polyphosphate Inhibitor UHRA. Blood 2017, 129 (10), 1368-1379, Kalathottukaren, M. T. et al. A Polymer Therapeutic Having Universal Heparin Reversal Activity: Molecular Design and Functional Mechanism. Biomacromolecules 2017, 18 (10), 3343-3358., and Kalathottukaren, M. T. et al. Comparison of Reversal Activity and Mechanism of Action of UHRA, Andexanet, and PER977 on Heparin and Oral FXa Inhibitors. Blood Adv. 2018, 2 (16), 2104-2114.) PolyPs are polymers of inorganic phosphates with densely packed anionic charges connected by high-energy phosphoanhydride bonds. PolyP plays an important role in blood clot formation by acting as a procoagulant stimulus at several enzymatic steps of the blood coagulation cascade, accelerating the clotting process. (Smith, S. A. et al. Polyphosphate Modulates Blood Coagulation and Fibrinolysis. Proc. Natd. Acad. Sci. U.S.A 2006, 103 (4), 903-908., Smith, S. A. and Morrissey, J. H. Polyphosphate Enhances Fibrin Clot Structure. Blood 2008, 112 (7), 2810-2816., Morrissey, J. H. and Smith, S. A. Polyphosphate as Modulator of Hemostasis, Thrombosis, and Inflammation. J. Thromb. Haemost. 2015, 13, S92-S97. Travers, R. J. et al. Polyphosphate, Platelets, and Coagulation. Int. J. Lab. Hematol. 2015, 37, 31-35., and Baker, C. J.; Smith, S. A.; Morrissey, J. H. Polyphosphate in Thrombosis, Hemostasis, and Inflammation. Res. Pract. Thromb. Haemost. 2019, 3 (1), 18-25.). PolyP is a potent accelerant of coagulation. (Morrissey, J. H. Tissue Factor: A Key Molecule in Hemostatic and Nonhemostatic Systems. Int. J. Hematol. 2004, 79 (2), 103-108, and Colman, R. W. and Schmaier, A. H. Contact System: A Vascular Biology Modulator With Anticoagulant, Profibrinolytic, Antiadhesive, and Proinflammatory Attributes. Blood 1997, 90 (10), 3819-3843. PolyP is thereby a therapeutic target. By targeting and inhibiting an accelerant instead of a key component of the clotting process, novel antithrombotics with lower risk of bleeding in comparison to current anticoagulation therapies may be developed. (Smith, 2006, ibid, Smith, 2008, ibid, Ruiz, ibid.) Naked cationic structures such as low molecular weight PEI and PAMAM dendrimers are effective at inhibiting polyP in vitro with strong electrostatic interactions between polyP and these polymers. (Smith, 2012, ibid, Jain, S. et al. Nucleic Acid Scavengers Inhibit Thrombosis without Increasing Bleeding. Proc. Natl. Acad. Sci. 2012, 109 (32), 12938-12943.) However, high toxicity has constrained acceptance of this approach.

Heparins, another class of polyanions with substantial therapeutic utility, are a polydisperse and heterogenous mixture of sulfated polysaccharides belonging to the glycosaminoglycan family of carbohydrates that are broadly used for their anticoagulant and antithrombotic properties. Heparin based anticoagulants, including unfractionated heparin (UFH), low-molecular weight heparins (LMWHs), enoxaparin, tinzaparin, dalteparin, and fondaparinux (a synthetic heparin pentasaccharide) are the most widely administered class of anticoagulants to the present. The antithrombotic activity of heparin arises from a specific pentasaccharide sequence that binds antithrombin (AT/AT-III), a serine protease inhibitor and an endogenous anticoagulant, thereby accelerating the inhibition of coagulation. A major adverse side effect of heparins is bleeding that causes increased mortality and hospitalization. Accordingly, heparin reversal is often required in patients under emergency conditions that require a safe and effective heparin antidote. In certain medical procedures such as cardiopulmonary bypass surgery and certain intravascular surgical procedures that require high doses of heparin anticoagulation, reversal of heparin after the surgery or procedure is routine to prevent hemorrhage.

Currently, the highly cationic polypeptide, protamine sulfate (PS), is the sole FDA-approved heparin antidote. PS functions via electrostatic binding to polyanionic heparin to form a stable ion pair that does not exhibit anticoagulant activity, leading to neutralization of the anticoagulant effects of heparin. PS has only limited efficacy in neutralizing LMWHs and has no reversal activity against fondaparinux, one of the main limitations of these otherwise superior anticoagulants to UFH. Furthermore, PS itself often lead to complications such as hypotension, excessive bleeding and hypersensitivity due to its lack of specificity. Despite being the conventional standard of care in numerous cardiac surgical procedures, heparin and PS must be carefully titrated to prevent severe bleeding. PS has unpredictable activity with a very narrow therapeutic window and has been linked to increased incidences of hypersensitivity, among other adverse outcomes.

Due to heparin's hemorrhagic and non-bleeding side effects, heparin analogues have been investigated in search of improved anticoagulant properties by developing synthetic heparinoids composed of functionalized polysaccharide backbones such as heparan sulfates, dermatan sulfates, chitosan sulfates, among many others. This work has led to a better understanding of the structural parameters that govern the properties of heparins, demonstrating that the anticoagulant and antithrombotic properties of heparinoids are directly affected by the structure of the polysaccharide macromolecules, the quantity and distribution of appended sulfated groups, and their molecular weight.

In parallel to efforts to develop viable heparin alternatives, work has focused on the development of heparin antidotes in view of the multiple limitations and complications associated with protamine. Approaches to the development of protamine alternatives include: protamine-like variants, peptide-based approaches, cationic polymers, polyvalent scaffold based-approaches, and dendritic universal heparin reversal agent (UHRA). Most of these approaches target heparin via an electrostatic binding mechanism and rely on polycationic functional groups to bind the polyanionic heparin macromolecules. However, each of these approaches has specific limitations. Synthetic structures carry an advantage over peptide-based approaches by eliminating the need for biologically sourced starting materials, and the associated risks of contamination and sensitivity-based reactions by the patient. Besides improved control over purity of the final materials, synthetic protamine alternatives also provide complete control of the final molecular structure which allows for facile modification of the heparin antidote to specifically tune the activity of the drug. The ability to tune these protamine alternatives has improved significantly as the effect of physicochemical properties on the biological activity are better understood. Although synthetic heparin antidotes are a promising route forward, the high quantity of positive charge these molecules must bear to effectively neutralize heparin has limited the utility of current synthetic protamine alternatives, with persistent side reactions and affinity to other negatively charged biological compounds that result in nonspecific interactions with blood and tissue in the body.

A third type of polyanion or polyanion assembly is extracellular nucleic acids (ecNAs) including extracellular DNA and neutrophil extracellular traps (NETs). Clinical and biochemical studies show that pathological thrombosis is initiated by the contact pathway of blood coagulation, with polyanion triggers comprising extracellular nucleic acids (ecNAs) and neutrophil extracellular traps (NETs). Although no therapies currently exist to target these polyanion triggers, blocking the triggers provides the opportunity to prevent thrombosis while sparing normal hemostasis.

Accordingly, there is a need for polyanion inhibitors that increase charge upon binding to target polyanion molecules that also exhibit minimal cationic charge at physiological pH, thereby providing reagents and therapeutics with enhanced biocompatibility and selective polyanion binding behavior.

### SUMMARY

Provided herein are compositions and methods to prevent and to treat thrombosis, to reverse the anticoagulant action of heparin, and to prevent and to treat the thrombotic and antifibrinolytic effect of nucleic acids. In particular, provided herein are compositions, methods, kits, systems and uses for switchable charge state multivalent biocompatible polycations for polyanion inhibition in blood.

In some embodiments, the present invention provides a method of preventing and/or treating thrombosis, comprising administering a macromolecular polyphosphate inhibitor (MPI) to a subject wherein the administering prevents and/or treats said thrombosis. In certain embodiments, the MPI comprises one or more cationic binding groups (CBGs,) and one or more biocompatible scaffolds. In further embodiments, the one or more CBGs is a linear alkyl amine. In still further embodiments, the one or more biocompatible scaffolds is a polyethylene glycol (PEG) scaffold and/or a polyglycerol scaffold. In particular embodiments, the MPI is MPI 8. In given embodiments, the subject is a human subject. In specific embodiments, the administering is parenteral administering.

In some embodiments, the present invention provides a method of reversing anticoagulation, comprising administering a macromolecular polyphosphate inhibitor (MPI) to a subject wherein the administering prevents and/or treats said anticoagulation. In certain embodiments, the anticoagulation is heparin anticoagulation, UFH heparin anticoagulation, enoxaparin anticoagulation, tinzaparin anticoagulation, dalteparin anticoagulation and fondaparinux anticoagulation. In further embodiments, the MPI comprises one or more cationic binding groups (CBGs,) and one or more biocompatible scaffolds. In still further embodiments, the one or more CBGs is a linear alkyl amine. In particular embodiments, the one or more biocompatible scaffolds is a polyethylene glycol (PEG) scaffold and/or a polyglycerol scaffold. In given embodiments, the MPI is MPI 2. In other embodiments, the subject is a human subject. In yet other embodiments, the administering is parenteral administering.

In some embodiments, the present invention provides a composition comprising a) one or more cationic binding groups (CBGs), b) one or more biocompatible scaffolds, and c) a pharmaceutically acceptable carrier. In certain embodiments, one or more CBGs is a linear alkyl amine. In other embodiments, the one or more biocompatible scaffolds is a polyethylene glycol (PEG) scaffold and/or a polyglycerol scaffold. In further embodiments, the composition is MPI 8. In still further embodiments, the composition is MPI 2. In particular embodiments, the present invention provides use of the preceding embodiments. In given embodiments, the present invention provides use of the preceding embodiments for the treatment of disease in a subject.

In some embodiments, the present invention provides a polymeric compound, comprising a) a hyperbranched polyglyercol core, b) a plurality of polyethylene glycol chains covalently attached to the hyperbranched polyglyercol core, and c) a plurality of linear alkylamine moieties covalently attached to the hyperbranched polyglyercol core. In certain embodiments, the linear alkylamine moieties have structures of formula (I):

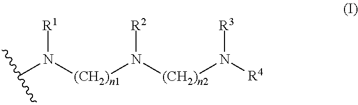

or a pharmaceutically salt thereof, wherein n1 and n2 are each independently selected from 2 and 3, R¹, R², R³, and R⁴ are each independently selected from C1-C3 alkyl; and

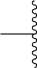

is the point of attachment to the hyperbranched polyglyercol core. In further embodiments, n1 and n2 are each 2. In other embodiments, n1 and n2 are each 3. In given embodiments, R¹, R², R³, and R⁴ are each methyl. In particular embodiments, the linear alkylamines have a structure selected from:

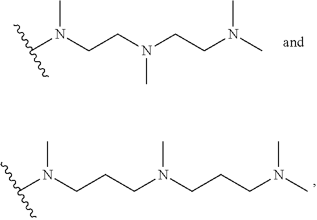 [SMILES: *N(C)CCN(C)CCN(C)C ; *N(C)CCCN(C)CCCN(C)C]

or a salt thereof.

In additional embodiments, the polymeric compound has a molecular weight of about 8 kDa to about 25 kDa, or about 10 kDa to about 23 kDa. In some embodiments, the core has a number average molecular weight of about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, or about 25 kDa, or any range therebetween. In specific embodiments, the polymeric compound has an average of about 10 to about 25 linear alkylamine moieties covalently attached to the hyperbranched polyglyercol core, e.g., an average of about 10, about 11, about 12, about 13, about 14, about 15, about 16, about 17, about 18, about 19, about 20, about 21, about 22, about 23, about 24, or about 25 linear alkylamine moieties covalently attached to the core.

### DESCRIPTION OF THE FIGURES

FIG. 1 shows the chemical structure of an inhibitor, binding groups and polyphosphate. a) Chemical structure of a generalized macromolecular polyphosphate inhibitor (MPI) in which the hyperbranched polyglycerol core is shown in black, the polyethylene glycol corona is shown in red, and the cationic binding groups are shown in blue. b) cationic binding groups in some embodiments of the present invention. c) Anionic polyphosphate structure showing the high charge density of this biopolymer.

FIG. 2 shows ¹H NMR taken on a 300 MHz spectrometer in CDCl₃ of HPG-mPEG₃₅₀. From top to bottom, each step of the post polymerization process of HPG-mPEG to MPI is shown.

FIG. 3 shows a characterization of MPI size and charge characteristics. a) Sample gel permeation chromatography trace for polymer size and dispersity characterization and b) sample conductometric titration of MPI to determine the average number of cationic binding groups (CBGs) loaded on the MPI. Voltage was measured as freshly standardized 0.1506 M NaOH was titrated into an acidified solution of MPI 9 at 25° C.

FIG. 4 shows an evaluation of free unbound MPI charge content. Potentiometric titrations were used to evaluate charge content of MPI library at 25° C. and 160 mM NaCl. a) A sample titration of 0.15 M NaOH into a solution of acidified MPI 3 while measuring the change in potential. b) The speciation plot calculated from titration MPI 3 with NaOH.

FIG. 5 shows binding curves and summary K_{d} values obtained for each MPI towards surface bound polyP by surface plasmon resonance (SPR). Two lengths of polyP were investigated. N=3 runs in running buffer of HEPES buffer with 140 mM NaCl at pH 7.4 and 25° C., error bars represent SD. Statistical significance for measured K_{d} was determined by comparing each MPI to the UHRA control using a one-way ANOVA followed by a Dunnett post hoc test. A significant increase K_{d} was observed in several MPI candidates, MPI 6, 7 and 9 compared to the buffer control. (****P<0.0001, ***P<0.001 *P<0.05) a) Sample binding curve of MPI 3 binding to long chain polyP, P1070. b) Sample binding curve of MPI 3 binding to medium chain polyP, P560. c) Summary of binding affinity of MPI library to long chain polyP. d) Summary of binding affinity of MPI library to medium chain polyP.

FIG. 6 shows summary binding curves for MPI binding to 3 surface-bound polyP sizes (LC polyP:P1070, MC polyP:P560) using surface plasmon resonance (SPR). Binding affinities were obtained from steady state affinity for each MPI for each run and then averaged over 3 runs. All concentrations were run at 25° C. in 20 mM HEPES running buffer with 140 mM NaCl, pH 7.4.

FIG. 7 shows binding curves obtained by isothermal titration calorimetry (ITC). a) Binding curve of small molecule CBG I, the same CBG appended onto MPI 3, binding to polyP (P700). Small molecule CBG I shows a weak binding curve. b) Binding curve of MPI 3 to polyP (P700), showing a strong binding curve with multivalent effects present upon binding. Sample titration is shown, while results were averaged of 3 repeated titrations. Heat of dilution has been subtracted. Tests were performed using HEPES buffer with 10 mM NaCl. All titrations were performed at 25° C.

FIG. 8 shows binding curves from ITC for MPIs that were evaluated for binding to polyP in different buffers with 10-150 mM NaCl as labelled. All experiments were conducted at pH 7.4 and 25° C. One representative titration is shown for each experiment while physical parameters were taken as the mean of 3 titrations each subtracted by their respective heat of dilution.

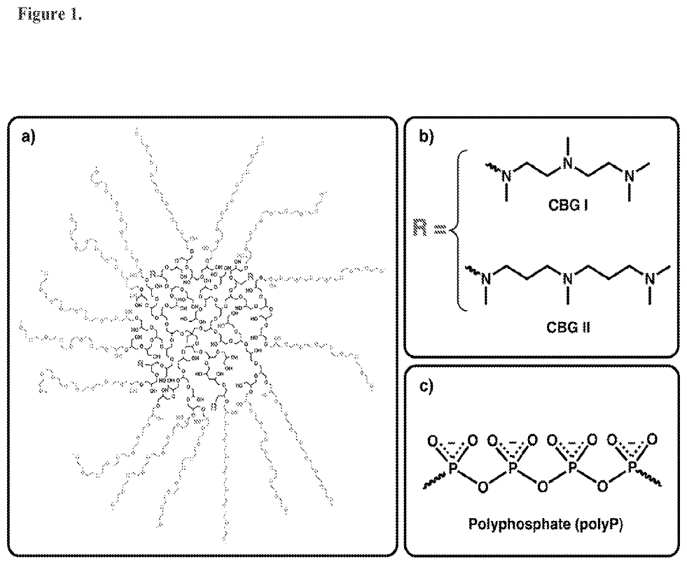

FIG. 1 shows binding curves obtained from ITC that compare MPI 3 binding to multiple sizes of polyP with strong binding affinities for all polyP sizes. a) Binding curve of MPI 3 to SC polyP (P45). b) Binding curve of MPI 3 to medium chain (MC) polyP (P75). c) Binding curve of MPI 3 to long chain (LC) polyP (P700). Sample titration is shown, with final fits reported in Table 5 as an average of 3 repeated titrations. The heat of dilution has been subtracted and was determined by using sodium phosphate buffer with 10 mM NaCl. All titrations were performed at 25° C.

FIG. 10 shows binding curves obtained using ITC for similar scaffold macromolecules with different CBG binding to polyP. Examples of ITC titrations are shown with MPIs and UHRA with different CBG structures binding to polyP (P75). a) Binding curve of MPI 1 to P75. b) Binding curve of MPI 5 to P75. c) Binding curve of MPI 3 to P75. d) Binding curve of UHRA-8 to P75. e) A slight decrease in the value of the dissociation constant was observed as the number of charges on the same scaffold increases. Sample titrations are shown, with reported K_{d} values representing the average of 3 repeat titrations ±SD. The heat of dilution has been subtracted and was determined using sodium phosphate buffer with 10 mM NaCl. All titrations were performed at 25° C.

FIG. 11 shows binding curves obtained using ITC that compare constructs made of the different scaffold sizes. Sample ITC titrations are shown with MPIs with different scaffold sizes binding to polyP (P45). a) Binding curve of MPI 9 to P45. b) Binding curve of MPI 5 to P45. c) Binding curve of MPI 7 to P45. d) Binding curve of MPI 3 to P45. e) A slight decrease in the obtained dissociation constants from a) to d) was observed as the scaffold size increased. Reported K_{d} values represent the average of 3 repeat titrations ±SD. The heat of dilution has been subtracted and was determined using sodium phosphate buffer with 10 mM NaCl. All titrations were performed at 25° C. Differences in the dissociation constants (a versus b and c versus d) are not statistically significant.

FIG. 12 shows enthalpy in different buffer environments compared to heat of ionization with recruitment of protons upon MPI binding to polyP. AHobserved obtained from binding MPI 3 and MC polyP (P75) using ITC200 in four buffers is plotted against reported ΔH_{ionization}, values for each buffer at pH 7.4, 150 mM NaCl and 25° C.

FIG. 13 shows binding properties observed in ³¹P NMR titration experiments, a) Overlay of ³¹P NMR spectra obtained for 25 molar ratios of CBG I/P45 showing a broad chemical shift which gradually shifts as the small molecule cationic binding group (CBG I) was added. b) Overlay of ³¹P NMR spectra obtained for 25 molar ratios of MPI 3/P45. As the bound phosphate peak increases, the unbound phosphate peak decreases, indicating a strong binding interaction between MPI 9 and P45. c) Change in chemical shift was plotted over molar ratio as CBG I was slowly titrated into a solution of polyP (P45) indicating a weak binding strength for this reaction. d) Change in chemical shift was plotted over molar ratio as MPI 3 was slowly titrated into a solution of polyP (P45) showing the strong binding curve for this interaction. e) Examples of spectra of MPI 3 added to P45 with a change in chemical shift from the internal phosphates, while the terminal phosphate peak remains relatively unshifted, indicating that terminal phosphates play a role in the binding event. The red curve shows unbound polyP while blue curve shows bound polyP to MPI.

FIG. 14 shows ³¹P NMR spectra showing the properties of phosphate nuclei during binding to a cationic binding partner. a) Overlay of ³¹P NMR spectra obtained for 25 molar ratios of MPI9/P45. b) Change in chemical shift was plotted over molar ratio as MPI 9 was slowly titrated into a solution of polyP (P45) showing the strong binding curve for this interaction.

FIG. 15 shows inhibition of polyP procoagulant effects on plasma clot times. The percentage of plasma clotting time was calculated from the clot time of the buffer control (100%) and the polyP control (0%). The plasma clotting assay was performed using human platelet-poor plasma (PPP), 20 donors pooled. N=3 biological replicates, 3 technical replicates each.

Negative control: PPP with tricine buffer. Positive control: PPP with polyP (700 monomer units, 20 μM monomer concentration). a) Actual clot time of positive and negative controls, showing a significant decrease in clot time upon addition of polyP. b)-c) Calculated percentages of clotting times to demonstrate polyP inhibition. The dotted line indicates the value for plasma incubated with buffer (i.e., buffer control).

FIG. 16 shows thrombin generation curves showing inhibition of long chain polyP by specific MPI compounds. Sample thrombin generation curves were pooled with normal plasma clotting initiated with and without added LC polyP. Addition of MPI (MPI 1, MPI 6 and MPI 8, respectively as shown) returns the clotting parameters of added 0.2 mM polyP to normal (no polyP added). Concentrations of MPI are indicated in the figure legend, in units of μg/mL.

FIG. 17 shows thrombin generation parameters showing LC polyP inhibition by MPI 1, MPI 6 and MPI 8. MPI 1, 6 and 8 show a dose-dependent inhibition of LC polyP in all thrombin generation parameters. N=3. Error bars represent SD. a) Lag time. b) Endogenous thrombin potential. c) Peak thrombin. d) Time to peak as a function of the MPI concentration. The dotted lines represent the positive and negative controls, as indicated.

FIG. 18 shows thrombin generation curves showing inhibition of short chain polyP by MPI compounds. Sample thrombin generation curves are FXII deficient plasma with clotting initiated with and without added SC polyP. Addition of MPI (MPI 1, MPI 6 and MPI 8, respectively are shown) returns the clotting parameters of added 5 μM polyP back to those seen when no polyP is added. Concentrations of MPI are indicated in the figure legend, in units of p g/mL.

FIG. 19 shows thrombin generation parameters showing short chain polyP inhibition by MPI compounds in FXII deficient plasma. MPI 1, 6 and 8, show a dose-dependent inhibition of LC polyP in all thrombin generation parameters. N=3. Error bars are SD. a) Lag time. b) Endogenous thrombin potential. c) Peak thrombin. d) Time to peak as a function of MPI concentration.

FIG. 20 shows properties of the MPI library in human plasma and whole blood in the absence of polyP. a) Tissue factor (TF) initiated plasma (20 donors pooled) clotting in a turbidimetric microplate clotting assay. Concentrations: MPI and protamine sulfate (PS) (protamine sulfate) [200 μg/mL], UHRA [75 μg/mL]. Lag time was measured and plotted as a ratio of lag time of buffer control. Absorbance measured at λ=405 nm. Clot time of MPI incubated plasma did not change significantly compared to UHRA at both concentrations tested. b) Percentage of activated platelets in platelet-rich plasma (PRP) incubated with either MPI, positive (TRAP), or negative (buffer) controls and measured by flow cytometry. MPI [100 μg/mL] did not induce platelet activation. PRP was isolated from citrated whole blood. Error bars indicate SD, n=2 donors. c)-d) Clotting parameters measured by rotational thromboelastometry. Fresh citrated blood was collected, and clotting initiated with CaCl₂) with or without added polycation. c) Clot time normalized to buffer control, showing effects of MPI and UHRA on whole blood clotting. d) Maximum clot firmness normalized to buffer control, showing effects of MPI and UHRA. a)-d) Error bars indicate standard error of the mean (SE, n=3 unless otherwise stated). Statistical significance was determined by comparing each MPI or polycation to the buffer control using a one-way ANOVA followed by a Dunnett post hoc test. A significant change was not observed for any MPI candidates compared to the buffer control. PolyP inhibitors such as UHRA or PS demonstrated significant deviation from clotting parameters of buffer controls. (****P<0.0001, *P<0.05, ns=not significant).

FIG. 21 shows that MPIs do not affect lag time in recalcification-triggered plasma clotting system. Re-calcification of human platelet poor plasma (PPP) in a turbidity microplate clotting assay. Lag time was measured and plotted as a ratio of lag time relative to the negative buffer control. Absorbance measured at A=405 nm. Data are all not significantly different from the negative buffer control, indicating no influence on plasma clot time. Error bars represent SD, n=3 biological replicates, 3 technical replicates each. a) 75 μg/mL b) 200 μg/mL.

FIG. 22 shows that MPI does not affect lag time in TF-initiated plasma clotting system. Clot times were measured on a coagulometer in a FXII-deficient plasma clotting system with clotting initiated by 100 μM tissue factor. N=3 experimental replicates, error bars indicate standard deviation. The negative control was a mixture of plasma, HBS and TF without MPI added, shown as grey dashed line with standard deviation as grey dotted lines. Clot times of plasma with added MPI [100 μg/mL] show no significant difference in clot time relative to the negative buffer control; most lie within a standard deviation of buffer control. Only UHRA-8 was significant with ****P<0.0001.

FIG. 23 shows that MPI compounds have no influence on thrombin generation in plasma. TF initiated human plasma (20 donors pooled) clotting parameters in a thrombin generation assay were acquired by calibrated automated thrombography. Buffer control represents plasma clotted with added buffer. MPI candidates when added in lieu of buffer show minimal effects on thrombin generation compared to UHRA-8. n=3 biological replicates, 3 technical replicates each. Error bars indicate SD. a) Lag time. b) Endogenous thrombin potential, ETP, demonstrating the total amount of thrombin generated. c) Time to peak, showing the time until peak thrombin is generated. d) Peak thrombin generated as a function of MPI or UHRA concentration.

FIG. 24 shows that specific MPI compounds have no influence on human platelet activation. Platelet activation in PRP was measured by flow cytometry. All MPIs tested do not induce a significant percentage of platelet activation compared to both plasma and buffer negative controls. Certain candidates (MPI 1, MPI 6, MPI 8) show platelet activation percentages in the same range as negative controls even up to high concentrations of 200 μg/mL. PRP was isolated from citrated whole blood. Error bars indicate SD, n=3 donors. ****P<0.0001, ns=not significant.

FIG. 25 shows that specific MPI compounds do not influence whole blood clotting. Rotational thromboelastometry was performed on fresh citrated human whole blood with clotting activated by CaCl₂). 100 μg/mL MPI was added to whole blood and buffer control was performed with each experiment, per donor. Error bars indicate SD, n=3 donors. **P<0.001, ***P<0.0005, ****P<0.0001. a) Clot time normalized to buffer control. b) Maximum clot firmness normalized to buffer control.

FIG. 26 shows that the effective dose of MPI 8 does not interfere with fibrin clot fiber thickness and morphology. Clots were made by incubating 2.6 mg/mL human fibrinogen in 2.5 mM CaCl₂) and 200 μM polyP (P700) or MPI 8 (20 μg/mL) when indicated, then clotting was initiated with 3 nM thrombin. Clots were allowed to mature for 1 hour and processed for SEM imaging. Images were acquired using a Helios 650 focused ion beam scanning electron microscope. Scanning electron micrographs of fibrin clots formed in the presence of a) buffer only, b) polyP only, c) MPI 8 only, and d) polyP with MPI 8 are shown. Clot images were taken at three magnifications 5000×, 10 000× and 25 000×. Images from 10 000× are depicted.

FIG. 27 shows that MPI 8 reduces both fibrin and platelet accumulation in mouse cremaster arteriole thrombosis model. Results were obtained from C57/BL6 mice in a cremaster arteriole thrombosis model showing accumulation of fluorescently labelled platelets post laser injury to untreated and treated mouse. N=8 injuries averaged per group.

FIG. 28 shows that MPI 8 delays time to occlusion in a mouse carotid artery model of thrombosis. In an arterial mouse thrombosis model, artery patency was monitored by Doppler flow probe. Injury was induced by topical application of FeCl₃ and patency is plotted versus time, comparing the saline control, MPI 8 and UHRA-10. a) At 100 mg/kg, MPI 8 is more effective than UHRA-10 by further delaying time to occlusion. b) At 200 mg/kg, MPI 8 and UHRA-10 have reached a similar level of patency, a maximum in this model by these inhibitors. c) At 300 mg/kg, MPI 8 shows a longer time to occlusion and increased patency compared to UHRA-10. All results shown are mean of n=8 mice.

FIG. 29 shows that MPI compounds of the present invention do not cause bleeding in mice at high dosages. The mouse tail bleeding model used C57/BL6 mice. Mice were injected in a blind study with up to 300 mg/kg of lead MPI candidates and demonstrated no increase in bleeding side effect, in contrast to mice administered with 200 U/kg of unfractionated heparin. Error bars indicate SD, n=8 mice per group. a) Bleeding times of mice administered with the MPI compounds up to 200 mg/kg. b) Hemoglobin lost by mice administered with the MPI compounds up to 200 mg/kg. c) Bleeding times of mice administered with MPI 8 at 300 mg/kg compared to UHRA-10 at the same concentration. d) Hemoglobin lost by mice administered with MPI 8 at 300 mg/kg compared to UHRA-10 at the same concentration.

FIG. 30 shows that mice administered with high doses of MPI 8 show no signs of acute toxicity. Female BALB/c mice in groups of 4 were administered either saline or MPI 8 at 2 doses, up to 500 mg/kg. After 24 hours, serum was collected from the sacrificed mice and analyzed for markers of toxicity. Mice injected with MPI 8 showed no increase in LDH, AST or ALT levels. a) Change in body weight. b) LDH activity. c) AST activity. d) ALT activity.

FIG. 31 shows that high doses [500 mg/kg] of MPI 8 were well tolerated in mice. Female BALB/c mice in groups of 4 were administered either saline or MPI 8, up to 500 mg/kg. Mice were monitored daily, and body weights were measured. After 15 days, serum was collected from sacrificed mice and analyzed for LDH levels. Mice injected with MPI 8 showed no significant change in body weight compared to mice injected with saline, and no increase in LDH levels. a) Change in body weight over 15 days. b) Quantity of daily change in body weight per cohort. c) LDH activity.

FIG. 32 shows a schematic representation of macromolecular polycationic inhibitor (MPI) binding to heparin. a) Overall MPI structure binding heparin to form a stable complex. b) Zoom in of charges on MPI and heparin shows that as the cationic charges on MPI initiate binding to the negative charges on heparin, changes in the electronic microstate of MPI induce a change in the susceptibility of protonation of MPI amines, resulting in a tunable protonation state capable of recruiting protons to successfully bind heparin. c) Chemical structure of MPI 2. d) Structure of hyperbranched polyglycerol, polymer core in MPI. e) Structure of polyethylene glycol, brush structure on MPI. f) Structures of cationic binding groups on HPG surface used to bind heparin.

FIG. 33 shows heparin reversal by an MPI library in human plasma. Clotting time from activated partial thrombin time (aPTT) was measured on a Stago ST4 coagulometer of heparinized human pooled normal plasma, showing complete heparin reversal by select MPI candidates. Error bars indicate SD with n=3 experimental replicates, 2 technical duplicates each. The dashed lines indicate the clotting time for plasma incubated with buffer or heparins. a) Concentration dependent MPI library reversal of 1 U/mL tinzaparin showing MPI 2 having complete inhibition of tinzaparin activity and no increase in clot time as concentration increases. b) Concentration dependent MPI library reversal of 4 U/mL UFH showing MPI 2 having complete inhibition of UFH activity and no increase in clot time as concentration increases. c) MPI library reversal of 1 U/mL tinzaparin showing that the MPI library completely reverses the effects of tinzaparin even at 12.5 μg/mL. d) MPI library reversal of 4 U/mL UFH showing MPI 2 completely reverses the effects of UFH even at 25 μg/mL demonstrating its high activity.

FIG. 34 shows a summary of MPI compounds' reversal activity against 0.5 U/mL UFH in a TF-triggered system on thrombin generation of pooled normal plasma using calibrated automated thrombography. N=1 experimental replicate, 2 technical replicates averaged. a) Sample thrombin curves with 0.5 U/mL UFH and 2.5 μg/mL MPI 2. b) Summary from total thrombin. c) Summary from lag time. d) Summary from peak thrombin. e) Summary from time to peak. f) Legend of MPI library and UHRA-8 tested.

FIG. 25 shows the effects of MPI 2 reversal of different heparins on thrombin generation. The thrombin generation assay was done by calibrated automated thrombography, showing the effects of MPI 2 reversal of multiple different heparin and heparin-type anticoagulants. a)-c) Thrombin curves with MPI 2 [2.5 μg/mL] and a) UFH [0.5 U/mL], b) enoxaparin [0.3 U/mL], c) fondaparinux [0.5 μg/mL], d)-f) Lag time of increasing concentrations of MPI 2 added to d) UFH [0.5 U/mL], e) enoxaparin [0.3 U/mL] f) fondaparinux [0.5 μg/mL]. Pooled normal plasma (PNP) with n=3 experimental replicates. Error bars indicate standard deviation (SD).

FIG. 36 shows thrombin generation by calibrated automated thrombography, showing the effects of MPI 2 reversal of UFH [0.5 U/mL], enoxaparin [0.3 U/mL] and fondaparinux [0.5 μg/mL]. The effects of heparin neutralization on thrombin generation are visualized via four parameters, with all four parameters returning to the buffer control. a) Lag time. b) Endogenous thrombin potential (ETP) indicating the total thrombin generated. c) Time to generation of peak thrombin. d) Maximum thrombin concentration.

FIG. 37 shows that MPI 2 has a larger therapeutic window than protamine sulfate and UHRA. Activated partial thromboplastin time (aPTT) with heparinized plasma was measured showing the efficacy of MPI 2 to reverse UFH [4 U/mL] and tinzaparin [1 U/mL] and its overdose effects, compared to UHRA and PS. MPI 2 inhibits the anticoagulation activity of both UFH and tinzaparin at lower concentrations than both UHRA and PS, 2 previously-used heparin antidotes. At higher concentrations of antidote, MPI 2 shows no adverse effects on aPTT clot time, contrary to protamine and UHRA.

FIG. 38 shows that MPI 2 fully reverses the effects of UFH in human whole blood at a lower concentration than UHRA. The effects of MPI 2 neutralization of UFH [0.5 U/mL] on whole blood clotting were assessed using rotational thromboelastometry (ROTEM). Buffer is used as a negative control, UFH [0.5 U/mL] as a positive control, and UFH [0.5 U/mL] with UHRA [100 μg/mL] are compared to UFH [0.5 U/mL] and MPI 2 [20 μg/mL]. Averaging 5 donors, clot times were obtained from ROTEM of fresh citrated whole blood for the same 4 experiments. Error bars indicate SD. ns=not significant, *P<0.05, ****P<0.0001. a) Sample thromboelastogram. b) Clot times.

FIG. 39 shows that MPI 2 exhibits compatibility with blood components and blood clotting in the absence of heparin. a) Percentage of activated platelets in platelet rich plasma (PRP) incubated with either MPI 2, positive or negative controls, measured by flow cytometry. MPIs do not induce platelet activation. Thrombin receptor activating peptide 6 (TRAP) was used as a positive control, and plasma and buffer were used as negative controls. PRP was isolated from citrated whole blood. Error bars indicate SEM, n=2 donors. ****P<0.0001, ns=not significant. b-d) Rotational thromboelastometry (ROTEM) of fresh citrated human whole blood clotting activated by recalcification. 100 μg/mL MPI was added to whole blood and buffer control was performed with each experiment, per donor. b) Sample thromboelastogram depicting the change in curve when UHRA was added compared to MPI. c) Clot time from ROTEM normalized to buffer control. d) Maximum clot firmness from ROTEM normalized to buffer control. Error bars indicate SEM, n=3 donors. **P<0.001, ***P<0.0005, ****P<0.0001

FIG. 40 shows that MPI 2 fully reverses the effects of UFH and enoxaparin (LMWH) in mice. C57/BL6 mice were injected with UFH [200 U/kg] followed by MPI 2 as a UFH antidote in a mouse tail bleeding model. On the left, the bleeding times of mice were recorded. On the right, hemoglobin loss was quantified by Drabkin's assay from the blood collected. Successful reversal of UFH activity was evaluated as a return to bleeding time and hemoglobin loss similar to saline control, as seen for 20 mg/kg MPI 2. Error bars indicate SD, n=8 mice per cohort. ns=not significant, *P<0.05 **P<0.005 ****P<0.0001.

FIG. 41 shows that MPI 2 does not affect normal bleeding in mice. C57/BL6 mice were injected with MPI 2 in a mouse tail bleeding model. UFH [200 U/kg] was used as a positive control. On the left, bleeding times of mice were recorded. On the right, hemoglobin loss was quantified by Drabkin's assay from the blood collected. Error bars represent SD, n=8 mice per cohort. ****P<0.0001, ns=not significant.

FIG. 42 shows screening and identification of nucleic acid inhibitors that prevent coagulation induced by nucleic acids. Inhibitors were incubated with 50% plasma spiked with 67.11 μg/mL of low molecular weight (LMW) poly IC. Clotting was triggered in a Stago Start 4 coagulometer by calcium, diluted re-lipidated tissue factor, and PCPS vesicles. The data are provided as percent neutralization with 0% being Poly IC with no inhibitor, and 100% being plasma with no poly IC and no inhibitor. The solid bars indicate 200 g/mL and striped bars indicate 100 g/mL of inhibitor concentration. Colors indicate different types of the R groups of the inhibitor.

FIG. 43 shows LMW poly IC (67 μg/mL) added to citrated plasma with different concentrations of MP3. Plasma clotting was triggered with calcium and diluted re-lipidated tissue factor. Clotting times were measured using Stago coagulometer.

FIG. 44 shows inhibition of contact pathway activation by HMW poly IC with MPI 3. Human plasma was incubated with high molecular weight (HMW) poly IC (67 μg/mL), and cleavage of substrate S2302 was measured at 405 nm in presence of different concentration of MPI 3.

FIG. 45 shows inhibition of thrombin generation triggered by HMW poly IC (67 μg/mL) by MPI-3 at different concentrations (0-100 microgram/mL). Thrombin generation was measured using Thrombinoscope's Calibrated Automated Thrombogram (CAT) assay. Plasma was incubated with HMINW poly IC and thrombin generation was triggered with PPP-low reagent, calcium and fluorogenic substrate. A. Thrombogram, B. Lag time, C. Endogenous thrombin potential (ETP). D. Peak thrombin.

FIG. 46 shows reversal of the anti-fibrinolytic effect of HMIW poly IC by inhibitor MPI 3. Plasma was incubated with HMINW poly IC and fibrinolysis was measured by triggering with thrombin, calcium and tissue plasminogen activator.

FIG. 47 shows results of MPI 3 administration in cecal ligation puncture (CLP) polymicrobial sepsis model in mice (D. Rittirsch et al. “Immunodesign of experimental sepsis by cecal ligation and puncture”. Nature protocols 4:1 (2009), 31-36., Toscano, M. et al. (2011). Cecal ligation puncture procedure. JoVE (Journal of Visualized Experiments), (51), e2860.) A 3-dose regimen of MPI-3 administration (subcutaneous) every 2 hours post-ligation as followed. The endpoint of the experiment was 8 hours post-surgery when the mice were euthanized, and blood was collected. (A) Shows levels of thrombin-antithrombin (TAT) complex in the mouse plasma. TAT complex was lower with the administration of MPI 3, but the difference was not statistically significant. (B) Shows levels of DNA in the mouse plasma. DNA levels were lower with inhibitor administration of MIP3 100 mg/kg. (C) and (D) show the thrombin peak height, and time to peak in mouse plasma thrombin generation assay. For statistical analysis, Brown-Forsythe and Welch ANOVA with two-stage Benjamini, Krieger, & Yekutieli procedure for controlling the false discovery rate (FDR) was used. A P value <0.05 was considered significant.

FIG. 48 shows plasma analysis of cytokines and chemokines in CLP mice treated with MPI 3. The heatmap shows a panel of cytokines and chemokines in mouse plasma. The color scheme is red and blue where red indicates high relative levels and blue indicates low relative levels.

FIG. 49 shows platelet activation when ADP and MPI 8 were mixed together. ADP (final [ADP]=40 μM) was mixed with human PRP (90 μL) for 15 minutes at 37° C., and then platelet activation was measured by measuring binding of anti-CD62P-PE antibody using flow cytometry (N=3 donors). There was no CD62P signal when MPI-8 at any concentration was incubated with PRP in the absence of ADP. Results show that the presence of MPI-8 does not alter the activity of ADP, indicating that MPI-8 does not inhibit ADP-mediated platelet activation.

FIG. 50 shows platelet activation when PRP was pre-incubated with MPI 8, and then platelets were activated by the addition of ADP. These data show that the presence of MPI-8 does not alter the activity of ADP, indicating that MPI-8 does not inhibit ADP-mediated platelet activation.

FIG. 51 shows representative images of e (green) and fibrin (red) hemostatic clot formation in response to a repetitive vascular injury of the saphenous vein.

FIG. 52 shows a quantitative analysis of the dynamics of platelet accumulation (left) and fibrin formation (right) in response to vascular injury in a saphenous vein.

### Definitions

To facilitate an understanding of the present disclosure, a number of terms and phrases are defined below:

As used herein, the term “alkyl” means a straight or branched saturated hydrocarbon chain, e.g., containing 1 to 6 carbon atoms (C₁-C₆ alkyl), 1 to 4 carbon atoms (C₁-C₄ alkyl), 1 to 2 carbon atoms (C1-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

As used herein, the term “hyperbranched polyglycerol” refers to a polymeric material prepared by ring-opening multibranching polymerization of glycidol using a 1,1,1-tris(hydroxymethyl)propane initiator.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., thromboembolism, atherosclerosis, cancer). Test compounds comprise both known and potential therapeutic compounds. A test compound can be determined to be therapeutic by screening using the screening methods of the present disclosure.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of a compound (e.g., a compound described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, or ex vivo.

As used herein, the term “target binding agent” (e.g., “target-binding protein” or protein mimetic such as an aptamer) refers to proteins that bind to a specific target. “Target-binding proteins” include, but are not limited to, MPIs, immunoglobulins, including polyclonal, monoclonal, chimeric, single chain, single domain, scFv, minibody, nanobody, and humanized antibodies.

As used herein, the term “toxic” refers to any detrimental or harmful effects on a cell or tissue as compared to the same cell or tissue prior to the administration of the toxicant.

As uses herein, “thrombosis” is the formation of a blood clot or “thrombus” within a blood vessel. In certain conditions, thrombosis arises from an inherited condition including, for example, factor V Leiden, prothrombin gene mutation, deficiencies of natural proteins that prevent clotting (for example, antithrombin, protein C and protein S), elevated levels of homocysteine, elevated levels of fibrinogen or dysfunctional fibrinogen (dysfibrinogenemia), elevated levels of factor VIII (and other factors including factor IX and XI), and abnormalities in the fibrinolytic system, including hypoplasminogenemia, dysplasminogenemia and elevation in levels of plasminogen activator inhibitor (PA-1). In other conditions, thrombosis is associated with acquired hypercoagulable conditions including, for example, cancer, medications used to treat cancer (e.g., taroxifen, bevacizumab, thalidomide and lenalidomide), trauma or surgery, central venous catheter placement., obesity, pregnancy, supplemental estrogen use including oral contraceptive pills (birth control pills), hormone replacement therapy, prolonged bed rest or immobility, heart attack, congestive heart failure, stroke and other illnesses that lead to decreased physical activity, heparin-induced thrombocytopenia (i.e., decreased platelets in the blood due to heparin or low molecular weight heparin preparations), lengthy airplane travel, antiphospholipid antibody syndrome, previous history of deep vein thrombosis or pulmonary embolism, myeloproliferative disorders such as polycythemia vera or essential thrombocytosis., paroxysmal nocturnal hemoglobinuria, inflammatory bowel syndrome, HIV/AIDS and nephrotic syndrome among other inherited and acquired disorders of coagulation.

### DETAILED DESCRIPTION OF THE DISCLOSURE

Provided herein are compositions and methods to prevent and to treat thrombosis, to reverse the anticoagulant action of heparin, and to prevent and to treat the thrombotic and antifibrinolytic effect of nucleic acids. In particular, provided herein are compositions, methods, kits, systems and uses for switchable charge state multivalent biocompatible polycations for polyanion inhibition in blood.

In experiments conducted in the course of development of certain embodiments of the present invention, biocompatible polycationic inhibitors were developed with high binding affinity to therapeutically relevant polyanions in blood (e.g., polyphosphates, heparins and extracellular nucleic acids) that provide selectivity and enhanced binding based on switchable protonation states and localized proton recruitment without the need for an external trigger. The polycations have low cationic charge states at physiological pH, while maintaining strong binding to different biologically relevant polyanions with high biocompatibility provided by polyglycerol and polyethylene glycol scaffolds. The cationic binding groups (CBGs) are based on pKₐ profiles of amines and spacing between the nitrogen atoms and are conjugated to the polymer scaffold. A library of polycations was synthesized using cationic ligands comprising novel combinations of strongly (pKₐ >8) and weakly (pKₐ ˜6-7) basic amine ligands presented on a semi-dendritic polymer scaffold. The protonation behavior of the new polycations has been characterized utilizing potentiometric titrations and speciation analyses. The binding affinities of the library of polycations has been confirmed using surface plasmon resonance (SPR) and isothermal titration calorimetry (ITC) analyses. The switchable protonation states of cationic ligands on these polycations have been established using potentiometry and ITC analyses, and ³¹P NMR analyses. Clotting and cell-based assays provide evidence of enhanced biocompatibility. We demonstrate the therapeutic utility of the polycation platforms of the present invention using three different targets: inhibition of polyphosphates, inhibition of extracellular nucleic acids, and reversal of the action of heparin. The therapeutic activities and safety of the molecules of the present invention are demonstrated both in vitro and in vivo. These results show that improved binding to polyanions has been achieved without excessive charge on the polycation structure at physiological pH via switchable protonation states, with improved biocompatibility and selectivity when compared to conventional polyP inhibitors. (Smith, 2012, ibid, Shenoi, ibid, Travers, ibid, Kalathottukaren and Abraham, 2017, ibid, Jain, ibid.)

Polyphosphate (PolyP) Inhibitors with Switchable Protonation State Prevent Thrombosis without Bleeding Risk

In experiments conducted in the course of development of certain embodiments of the present invention, optimization of polycationic drug systems and reduction of toxicity has been demonstrated. Conventional systems rely on a stealth cationicity to shield their polycationic character until they have reached the target site. Selective local unleashing of polycationic substructures typically requires a trigger, either chemical pH changes which are useful to target tumors (Kato, Y. et al., Acidic Extracellular Microenvironment and Cancer. Cancer Cell Int. 2013, 13 (1), 89.) inflammatory tissue (Trevani, A. S. et al. Extracellular Acidification Induces Human Neutrophil Activation. J. Immunol. 1999, 162 (8), 4849-4857., and Dubos, R. J. The Micro-Environment of Inflammation or Metchnikoff Revisited. Lancet 1955, 1-5.0, lipid nanoparticle-siRNA delivery systems (Semple, ibid, Allen, ibid), or physical triggers such as light (Hu, L.-C. et al. Light-Triggered Charge Reversal of Organic-Silica Hybrid Nanoparticles. J. Am. Chem. Soc. 2012, 134 (27), 11072-11075.), temperature (Don, T.-M. et al. Temperature/PH/Enzyme Triple-Responsive Cationic Protein/PAA-b-PNIPAAm Nanogels for Controlled Anticancer Drug and Photosensitizer Delivery against Multidrug Resistant Breast Cancer Cells. Mol. Pharm. 2017, 14 (12), 4648-4660.), or magnetism (Wong, J. E. et al. Dual-Stimuli Responsive PNiPAM Microgel Achieved via Layer-by-Layer Assembly: Magnetic and Thermoresponsive. J. Colloid Interface Sci. 2008, 324 (1), 47-54.) While such systems find utility for their specific drug targets, they require an external trigger to fulfill their intended task. In some embodiments of the present invention, the trigger for increasing the cationic charge is the target binding site itself: the intended polyanion.

Using an adaptable design approach, we developed polyP inhibitors with improved activity and substantially enhanced biocompatibility. The improvements were achieved via a two-pronged approach: 1) using a series of cationic binding groups with switchable protonation states, and 2) a biocompatible scaffold. The selective design of cationic binding groups structures for their amine pKₐ values, charge spacing to match their targeted polyanion and length of linker which may affect their flexibility. By embedding the cationic binding groups to a biocompatible scaffold known to preclude non-specific interactions, polycations are generated with low charge density compared conventional polycation inhibitors of polyP (Smith, 2012, ibid, Travers, ibid, Kalathottukaren and Abraham, ibid), while further preventing nonspecific binding via steric repulsion of larger polyanionic macromolecules with lower charge density. Furthermore, this approach facilitated specific tuning of the macromolecular polyanion inhibitor (MPI) efficacy by synthesis of a library of compounds by varying backbone size, CBG structure, and the quantity and density of charge.

The library of potential polyP inhibitors was characterized by several methods to select optimal inhibitors. Assessment of the number of cationic binding groups per MPI was accomplished via conductometric titration, a factor governing the biocompatibility of the inhibitors, coupled with determination of their protonation behavior measured via potentiometric titration. This measurement provides an approximation of the state of charge of each MPI candidate examined. With the microstructure of the MPIs generated, we determined the effect of empirically measured characteristics on the binding behavior of the MPIs.

MPI binding behavior with polyP, an important newly identified antithrombotic target, was characterized via two principal techniques: 1) high-throughput surface plasmon resonance experiment; and 2) isothermal titration calorimetry. The characterization demonstrated that MPIs generated demonstrate strong binding to multiple lengths of polyP. While MPIs carry significantly less charge than previous universal heparin reversal agent (UHRA) inhibitors (ca. 50%-85% of UHRA charge), the binding and corresponding K_{d} did not suffer a significant loss. All measured binding affinities were on the sub-micromolar range with minimal differences. The strongest binding behavior is observed with the more charged Me₆TREN ligand, a ligand shown in further experiments to demonstrate lower biocompatibility due to a higher state of charge under physiological conditions. Of two CBGs, CBG II showed slightly enhanced binding behavior attributed to an inter-charge spacing that more closely matches that of the polyP partner. Isothermal titration calorimetry was then performed on a subset of the MPIs. For all binding pairs of MPI and polyP tested, a one-to-one stoichiometry was found, indicating a stable bound complex with no evidence of precipitation, a common occurrence in polycationic therapeutics. (Bernkop-Schnurch, ibid.) While the binding affinity increased with an increase in charge density on the MPI, the IC₅₀ remained similar over the series of inhibitors indicating that only a subset of the charges on the MPI participate in binding.

Using CBGs with multiple protonatable sites, MPIs were generated that exhibited a sufficiently low quantity of charge and charge density at physiological pH to remain biocompatible while maintaining the capacity to adopt a more highly charged state when bound to polyP. This switchable protonation behavior is analogous to similar processes observed in protein-ligand binding events in which the microenvironment of the binding pocket is more stable with a protonated ligand and therefore the ligand adopts a protonated state, recruiting a proton from the surroundings. (Neeb, M. et al. Chasing Protons: How Isothermal Titration Calorimetry, Mutagenesis, and PKa Calculations Trace the Locus of Charge in Ligand Binding to a TRNA-Binding Enzyme. J. Med. Chem. 2014, 57 (13), 5554-5565., Antosiewicz, J. et al. The Determinants of PKas in Proteins. Biochemistry 1996, 35 (24), 7819-7833., and Graffner-Nordberg, M. et al. Computational Predictions of Binding Affinities to Dihydrofolate Reductase: Synthesis and Biological Evaluation of Methotrexate Analogues. J. Med. Chem. 2000, 43 (21), 3852-3861.) Proton recruitment during the binding process is illustrated in FIG. 9, noting that under physiological conditions, the CBGs adopt a charge state that corresponds to between one and two protonated amines per ligand. When exposed to the highly anionic microenvironment surrounding the polyP partner, the CBGs adopt a more charged state, exhibiting two cationic residues per ligand, resulting in higher charge density on the MPI. The recruitment process underlies the switchable protonation behavior of MPIs. Once one cationic residue has bound to the polyP, additional binding events are energetically favorable, resulting in strong MPI-polyP binding from the highly charged MPI in the bound state. While an understanding of the mechanism is not necessary to practice the invention, and the present invention is not limited to any particular mechanism, the multivalent presentation of charges on both MPI and polyP from the initial cationic residues binding leads to strong avidity effects, satisfying the energetic requirement for the proton recruitment to achieve a stable complex at equilibrium, further evidenced by the classic enthalpy-entropy compensation observed from ITC. (Olsson, T. S. G. et al. Extent of Enthalpy-Entropy Compensation in Protein-Ligand Interactions. Protein Sci. 2011, 20 (9), 1607-1618.) Accordingly, MPI designs of the present invention are tailorable to high charge density polyanions by matching the charge map of its target polyP.

The enhanced strength of the MPI-polyP bound state over more simplified small molecule cationic inhibitors (e.g., CBG I) with polyP was confirmed via ³¹P NMR studies that demonstrated the equilibrium binding process observed for small molecule inhibitors that is not observed with the MPI. Instead, the high strength MPI-polyP bond results in distinct peaks for bound and unbound phosphates in this NMR experiment as the MPI is too tightly bound to rapidly equilibrate. Furthermore, observation of the phosphorus in polyP over the course of binding to MPI indicated no appearance of monophosphate, diphosphate or triphosphate peaks created, indicating that MPI are binding to the negative charges on the polyP chain without hydrolyzing polyP into smaller fragments. These observations support a further advantage of MPI over therapeutic enzymes that degrade polyP (Labberton, L. et al. Neutralizing Blood-Borne Polyphosphate in Vivo Provides Safe Thromboprotection. Nat. Commun. 2016, 7, 12616.). Specific enzymes may degrade phosphate units from other sources of phosphate in the body while the specificity of MPI is inherent to the polyanionic character of polyP.

The switchable protonation states achieved with the newly developed CBGs allow for precise specificity towards polyP while minimizing nonspecific interactions between the MPIs and negatively charged biomolecules, thereby resulting in enhanced biocompatibility. Enhanced biocompatibility was demonstrated by a series of tests to measure the effect of the polycationic drug candidates on hemostasis. Pooled plasma was treated with the MPI candidates as well as a buffer control to demonstrate no change in the lag time prior to clotting, showing that MPI addition alone does not have adverse effects on clotting behavior. Cationic therapeutics such as protamine sulfate performed considerably worse than MPI candidates, with a near 3-fold increase in clot time compared to that of the buffer control. This drawback is evidenced by the bleeding complications in patients who have been administered protamine sulfate without careful monitoring and titration. (Cobel-Geard, R. J. and Hassouna, H. I. Interaction of Protamine Sulfate with Thrombin. Am. J Hematol. 1983, 14 (3), 227-233., Wolberg, A. S. Thrombin Generation and Fibrin Clot Structure. Blood Rev. 2007, 21 (3), 131-142., and Ni Ainle, F. et al. Protamine Sulfate Down-Regulates Thrombin Generation by Inhibiting Factor V Activation. Blood 2009, 114 (8), 1658-1665.) Moreover, when used as a polyP inhibitor, MPIs of the present invention exhibit potent inhibition of procoagulant polyP activity in human plasma, a representation of their behavior in vivo as an antithrombotic.

To test the efficacy of each MPI as an inhibitor of bacterial-sized polyP and platelet-sized polyP in combining the IC₅₀ values and the inherent influence of MPI on tissue factor pathway-initiated clotting, MPI 1, MPI 6 and MPI 8 were tested as antithrombotic therapeutics. The IC₅₀ values did not correlate directly with increased charge density. Increasing charge density on certain MPI molecules (e.g., MPI 1, 2 and 3 with increasing charge density respectively) did not result in an increase in the efficacy of binding polyP, indicating binding behavior in which not all cationic ligands are necessarily interacting with the polyP, and highlighting the importance of CBG selection to optimally match the structure of polyP. MPI 1, 6, and 8, exhibit properties of a preferred polyP inhibitor with antithrombotic applications in vitro with minimal influence on clotting in the absence of added polyP. These MPIs provide a significant advantage in their ability to target multiple therapeutically relevant sizes of polyP by taking advantage of the strength of multivalent interactions between the two polyions, while mitigating nonspecific interactions with the reduced quantity of positive charge on MPI. Compared to other methods of inhibiting the effects of polyP such as enzymatic approaches, the MPIs of the present claims provide a significant advantage. MPIs of the present invention reverse the procoagulant effects of multiple sizes of polyP in keeping with the multivalency presented. This indicates that MPIs of the present invention target bacterial and platelet polyP without interference from other phosphate-containing compounds, in comparison to enzymatic degradation approaches that may cleave phosphates from other compounds. (Labberton, ibid.)

MPIs of the present invention demonstrated no interference with blood components including platelets, and were tested in more complex systems (e.g., whole blood) to probe whether these compounds have adverse effects on whole blood clotting. Whole blood is a preferred model system because it includes a full spectrum of biomolecules and cells that MPIs will encounter when used as an antithrombotic therapeutic in humans. Human whole blood is composed of multiple anionic components that typically lead to nonspecific interactions with unprotected polycations that have been previously investigated as drug candidates. Specifically, cationic PAMAM dendrimers and PEI have been shown to interact with and activate platelets and induce cell toxicity. (Pretorius, E. et al. Blood Clot Parameters: Thromboelastography and Scanning Electron Microscopy in Research and Clinical Practice. Thromb. Res. 2017, 154, 59-63.) MPIs of the present invention differ from conventional cationic polymers because they have minimized charge density while in circulation. This feature combined with cationic ligands via a carefully designed protonation state which is paired with a highly biocompatible polymer scaffold and PEG corona, provide new-generation inhibitors with significantly improved safety. The result of these measures has enhanced whole blood compatibility that is unrivaled by other polycationic drug candidates. (Kalathottukaren and Abraham, ibid.) The minimized effect on whole blood clotting is observed by the minimized change in clot time relative to buffer control in whole blood, measured via ROTEM, as well as by minimized change in maximum clot firmness upon MPI addition. Prior polyP inhibitors, UHRA-8 and UHRA-10, exhibited a significant increase in clot time and a decrease in maximum clot firmness, indicating that both these compounds interfere with the clotting process and the final clot stability. Because human whole blood more closely represents components that MPI may encounter in a therapeutic setting, the compatibility with whole blood clotting denotes improved biocompatibility.

The potential effect of the MPI 8 on the stability of the formed fibrin clots was assessed to observe potential changes in clot morphology and microstructure. The participation of MPIs of the present invention in clot formation and its resulting effects can be observed directly on the thickness and morphology of the generated fibrils as fibrinogen is converted to fibrin. Frequently, thickening of fibrin fibrils causes instability in the final clot, resulting in increased susceptibility to clot lysis which can in turn increase the risk of bleeding as a direct result of the abnormal clot structure. (Shenkman, B. et al. In Vitro Evaluation of Clot Quality and Stability in a Model of Severe Thrombocytopenia: Effect of Fibrinogen, Factor XIII and Thrombin-Activatable Fibrinolysis Inhibitor. Blood Transfus. 2014, 12 (1), 78-84.) Based on SEM measurements, a minimal difference was observed between clots generated in the presence of MPI 8 compared to clots generated in buffer control, further highlighting the minimal nonspecific interactions between polycations of the present invention and blood components that would potentially cause undesired activity in vivo. This was further evidenced by clots formed in the presence of conventional anticoagulants, that demonstrated increased clot turbidity attributed to increased fibril thickness that result in increased sensitivity of the clots formed to lysis. (Nenci, G. et al. Fibrin Clots Obtained from Plasma Containing Heparin Show a Higher Sensitivity to T-PA-Induced Lysis. Blood Coagul. Fibrinolysis 1992, 3 (3), 279-286., Collen, A. et al. Unfractionated and Low Molecular Weight Heparin Affect Fibrin Structure and Angiogenesis in Vitro. Cancer Res. 2000, 60 (21), 6196-6200.).

PolyP itself increases fibrin fiber thickness, leading to clots that are more resistant to fibrinolysis (Morrissey, ibid.) While the amount of polyP present in the physiological setting (up to approximately 3 μM in whole blood following complete platelet activation) may show less pronounced effects, changes in fibril thickness have shown significant thrombotic risk. (Wolberg, A. S. et al. Prothrombin Results in Clots with an Altered Fiber Structure: A Possible Mechanism of the Increased Thrombotic Risk. Blood 2003, 101 (8), 3008-3013., and Cooper, A. V. et al. Fibrinogen Gamma-Chain Splice Variant Γ′ Alters Fibrin Formation and Structure. Blood 2003, 102 (2), 535-540.) Notably, the MPI 8 reverses the polyP effect without altering the final clot structure, leaving a clot with fibrin fibrils similar to the buffer control, and providing a strong indication that MPIs of the present invention may reverse polyP activity without causing adverse effects on the final clot. In some embodiments of the present invention, therapeutics that modulate the degree of interaction between polyP and the fibrin clot directly modulate the final clot structure, its stability and lysis, and the physical properties of the resulting clot.

To test the properties and function of the MPIs of the present invention in vivo, the antithrombotic activity of MPIs of the present invention were tested in two mouse thrombosis models. Two additional mouse models were used to determine whether the MPIs produced undesired bleeding effects using a bleeding and toxicity model. The activity of the MPIs of the present invention were tested for antithrombotic activity via the rate and quantity of platelet and fibrin accumulation at the site of injury upon laser injury to cremaster arterioles. Mice administered with MPI 1 and MPI 6 demonstrated significantly less platelet accumulation. Mice administered 100 mg/kg MPI 8 demonstrated significantly less fibrin and platelet accumulation upon injury compared to the mice administered with saline. The ability of MPI 8 to prevent thrombus formation in a carotid artery model was tested wherein artery patency is monitored by Doppler flow probe following topical application of FeCl₃, inducing injury. Percent patency was monitored over time (30 minutes) and mice were administered with either MPI 8, saline or UHRA-10 (FIG. 28). MPI 8 demonstrated superior performance to UHRA-10, delaying the time to occlusion (FIG. 28a). There was no significant dose response shown by MPI 8 at higher doses. The observed activity is superior to previous generation polyP inhibitors including UHRA-10. The patency shown by MPI 8 in this model indicates that inhibiting only polyP may not result in 100% patency unlike high dose anticoagulants such as heparin which in turn results in bleeding. (Travers, ibid.) These data are consistent with a recent report of enzymatic cleavage of polyP, but that approach was associated with adverse side effects including the degradation of other critical small molecular polyphosphates. (Labberton, ibid.) A further advantage of MPIs of the present invention is the absence of adverse effects that are present with the use of other reported polycations. For example, other polycations such as PAMAM dendrimer, PEI and polymyxin B have been examined in this model and showed less than 30% final patency. (Smith, 2012, ibid.) Additionally, these polycations have been shown to be toxic. (Moreau, E. et al. Biocompatibility of Polycations: In Vitro Agglutination and Lysis of Red Blood Cells And In Vivo Toxicity. J. Drug Target. 2002, 10 (2), 161-173.)

Experiments conducted in the development of the present invention indicate that MPI 8 does not influence normal hemostasis processes and is less likely to be associated with bleeding as compared to other antithrombotic agents. MPI 8 does not induce bleeding even at the high concentrations of 300 mg/kg, whereas previous studies have shown UHRA-10 does induce bleeding in mice at lower concentrations. (Travers, ibid.) The mean of bleeding times after administration of UIRA-10 was not significantly different from the saline control; however, the strong deviation (coefficient of variation of 49%) in the bleeding times indicates an erratic dose behavior for this compound. The more predictable bleeding time for MPI 8 similar to the saline control further indicates its advantages. MPIs of the present invention provide marked safety based on both acute and chronic toxicity studies in mice. These data show that even at the very high dose of 500 mg/kg, MPI 8 did not induce any changes in the enzyme levels, which would indicate cell/tissue damage, indicating that MPI 8 is well tolerated. These data also indicate that MPI 8 is the most non-toxic polycation reported in the literature to date. Compared to polycationic PS, MPI 8 provides much improvement over the toxic complications with a lethal dose of PS of 30 mg/kg in mice. (Shenoi, R. A. et al. Affinity-Based Design of a Synthetic Universal Reversal Agent for Heparin Anticoagulants. Sci. Transl. Med 2014, 6 (260), 260ra150.) In turn, PS is cytotoxic to cells and potentiates tissue damage. (Sokolowska, E. et al. The Toxicology of Heparin Reversal with Protamine: Past, Present and Future. Expert Opin. Drug Metab. Toxicol. 2016, 12 (8), 897-909.) Even small doses may result in capillary thrombosis and severe damage to the glomerular and tubular epithelium (located in the kidney), (Messina, A. et al. Protamine Sulphate-Induced Proteinuria: The Roles of Glomerular Injury and Depletion of Polyanion. J. Pathol. 1989, 158 (2), 147-156.), and heparin-protamine complexes cause distinct hemorrhage and pulmonary edema in the lungs of rats. (Cook, J. J. et al. Platelet Factor 4 Efficiently Reverses Heparin Anticoagulation in the Rat without Adverse Effects of Heparin-Protamine Complexes. Circulation 1992, 85 (3), 1102-1109.) Compared to other cationic polymers, PAMAM and polypropylenimine (PPI) from generations G3 to G5 have shown to be highly cytotoxic to cells. (Bodewein, L. et al. Differences in Toxicity of Anionic and Cationic PAMAM and PPI Dendrimers in Zebrafish Embryos and Cancer Cell Lines. Toxicol. Appl. Pharmacol. 2016, 305, 83-92.) In particular, high molecular weight polycations such as dendritic and hyperbranched polylysine of >20 kDa demonstrate acute cytotoxicity via direct cell membrane disruption. (Kadlecova, Z. et al. Comparative Study on the In Vitro Cytotoxicity of Linear, Dendritic, and Hyperbranched Polylysine Analogues. Biomacromolecules 2012, 13 (10), 3127-3137.) Polycations such as poly(L-lysine) and polydiallyldimethylammonium chloride (PDDAC) also cause acute toxicity in animals. At lethal doses, mice showed respiratory distress and convulsive movements before dying and the maximal non-toxic dose was low: 10 mg/kg for 24 kDa poly(L-lysine); 5 mg/kg for 124 kDa poly(L-lysine); and only 4 mg/kg for PDDAC. (Moreau, ibid.) These data provide a clear indication of the high biocompatibility of MPIs of the present invention in contact with more complex systems in the setting of antithrombotic therapy.

Compared to current polyP inhibitors, MPIs of the present invention provide multiple advantages. Conventional polycations (e.g., poly(lysine), PEI and PAMAM) are cytotoxic and cause adverse effects through their strong nonspecific interactions with other blood components. (Jones, C. F. et al. Cationic PAMAM Dendrimers Aggressively Initiate Blood Clot Formation. ACS Nano 2012, 6 (11), 9900-9910., and Hu, et al. Cardiovascular Toxicity Assessment of Poly (Ethylene Imine)-Based Cationic Polymers on Zebrafish Model. J. Biomater. Sci. Polym. Ed. 2017, 28 (8), 768-780.) Peptide-based approaches such as poly-L-lysine may alter fibrin fibril thickness resulting in an increased risk of thrombosis. (Shenkman, B. et al. In Vitro Evaluation of Clot Quality and Stability in a Model of Severe Thrombocytopenia: Effect of Fibrinogen, Factor XIII and Thrombin-Activatable Fibrinolysis Inhibitor. Blood Transfus. 2014, 12 (1), 78-84.) Another approach to reverse the effects of polyP includes uses polyphosphatase-based approaches, for example, use of a polyP degrading enzyme such as recombinant Escherichia coli exopolyphosphatase (PPX) and a PPX variant lacking domains 1 and 2 (PPX_D12, which binds polyP but does not degrade it). (Labberton, ibid.) However, degradation of polyP by the PPXenzyme may take substantial time to act, and also requires that the terminal phosphate(s) of polyP are not complexed with proteins or other substances. MPIs of the present invention do not have these adverse interactions, but rather demonstrate hemocompatibility and a high dose tolerance in mice. Because MPIs of the present invention inhibit polyP through an electrostatic neutralization without degrading polyP, they do not exhibit nonspecific interactions with critical small-molecule, phosphate-containing compounds.

Conventional anticoagulants such as heparin or LMWHs are effective at preventing venous thromboembolism (VTE) and therefore often administered to patients that are at high risk for VTE. (Schünemann, H. J. et al. American Society of Hematology 2018 Guidelines for Management of Venous Thromboembolism: Prophylaxis for Hospitalized and Nonhospitalized Medical Patients. Blood Adv. 2018, 2 (22), 3198-3225) who may further experience bleeding issues. Direct oral anticoagulants (DOACs) have been developed, which target factor Xa or thrombin, downstream enzymes in the coagulation cascade. (Weitz, J. I. and Fredenburgh, J. C. Factors XI and XII as Targets for New Anticoagulants. Front. Med. 2017, 4, 19.) Since 2010, DOACs have been FDA-approved and were considered to have good overall safety profiles and low bleeding risk. However, recent studies show that DOACs may not provide a lower bleeding risk to LMWH, highlighting the continued need for a safe and effective antithrombotic agent. (Tao, D. L. et al. The Efficacy and Safety of DOACs versus LMWH for Cancer-associated Thrombosis: A Systematic Review and Meta-analysis. Eur. J Haematol. 2020, 105 (3), 360-362., and Neumann, I. et al. DOACs vs LMWHs in Hospitalized Medical Patients: A Systematic Review and Meta-Analysis That Informed 2018 ASH Guidelines. Blood Adv. 2020, 4 (7), 1512-1517.) To the present, there is no effective anticoagulant drug available that does not present bleeding risks, with targeting newly identified polyanions as an attractive approach. (La, ibid.) Targeting polyP for its ability to prevent the acceleration of coagulation provides a pathway for the development of safer antithrombotic strategies. (Weitz, ibid.) The current absence of available therapeutics to target new pathologies such as those involving polyP or other polyanions underscores the value of MPIs of the present invention. (La, ibid, Weitz, ibid, Connors, J. M. and Levy, J. H. COVID-19 and Its Implications for Thrombosis and Anticoagulation. Blood 2020, 135 (23), 2033-2040., and Rangaswamy, C. et al. Polyanions in Coagulation and Thrombosis: Focus on Polyphosphate and Neutrophils Extracellular Traps. Thromb. Haemost. 2020.) Compared to conventional technologies (Smith, 2012, ibid, Jain, ibid, Labberton, ibid.) MPIs of the present invention provide therapeutics with nontoxic thromboprotection without bleeding risk and toxicity.

Other approaches to the development of biocompatible, effective cationic therapeutic agents including PEGylation (Veronese, F. M. Peptide and Protein PEGylation: A Review of Problems and Solutions. Biomaterials 2001, 22 (5), 405-417., and Turecek, P. L. et al PEGylation of Biopharmaceuticals: A Review of Chemistry and Nonclinical Safety Information of Approved Drugs. J. Pharm. Sci. 2016, 105 (2), 460-475.), HPG-conjugation (Frey, H. and Haag, R. Dendritic Polyglycerol: A New Versatile Biocompatible Material. Rev. Mol. Biotechnol. 2002, 90 (3), 257-267., Kurniasih, I. N. Synthesis and Transport Properties of New Dendritic Core-Shell Architectures Based on Hyperbranched Polyglycerol with Biphenyl-PEG Shells. New J. Chem. 2012, 36 (2), 371-379., Paulus, F. et al Structure Related Transport Properties and Cellular Uptake of Hyperbranched Polyglycerol Sulfates with Hydrophobic Cores. Polym. Chem. 2014, 5 (17), 5020-5028., and Abbina, S. et al. Hyperbranched Polyglycerols: Recent Advances in Synthesis, Biocompatibility and Biomedical Applications. J. Mater. Chem. B 2017, 5 (47), 9249-9277.), and the use of a polymeric shielding layer, do not comprise switchable polycationic inhibitors that do not require an extrinsic change in conditions, such as a change in the environmental pH or temperature. In experiments conducted in the development of certain embodiments of the present invention, we discovered a series of cationic binding groups with protonation states that vary significantly with shifts in dielectric strength at physiological pH. We further show that polycationic therapeutics adopt a highly charged state during binding while adopting a minimally charged state during free circulation. We developed highly effective polyP inhibitors that exhibit high polyP binding activity but with minimal non-specific binding that is otherwise endemic to polycationic macromolecules. Using this design methodology, we demonstrate that targeted polycationic inhibitors provide a new class of therapeutics that minimizes risks attendant to conventional drugs and technologies with high biocompatibility.

Through comprehensive physical characterizations, we have demonstrated new methods to design, manufacture and administration of new, safe and effective macromolecular polyanion inhibitors. Using specifically designed cationic binding groups, we show that MPIs with switchable protonation behavior of the present invention target polyP without undesirable nonspecific interactions in blood. The synthetic route to the MPIs of the present invention allows for rapid generation of a library of inhibitors available for high throughput screening and optimization. ITC and SPR experiments demonstrate that the new polyP inhibitors possess surprisingly high binding affinity towards diverse biologically relevant chain lengths of polyP in view of a low charge density at unbound physiological conditions. In turn, experiments in human plasma demonstrate that MPIs of the present invention provide high polyP inhibition activity in reversing the procoagulant behavior of polyP at sub-micromolar concentrations. The new combination of weakly acidic amine structures on a biocompatible scaffold maintains specificity towards polyP with a change in protonation state upon inhibitor binding. Cationic structures of the present invention demonstrate selectivity for polyP with no interference with other anionic blood components such as proteins and platelets which are activated by other cationic compounds. MPIs of the present invention exhibit minimal cationic charge density at physiological pH that increases significantly upon binding to polyP with improved biocompatibility and binding behavior.

### Protonation of Cationic Ligands in the Design of Safe and Effective Universal Heparin Antidotes

As the need for a safe and effective heparin reversal agent expands, efforts have focused on several possible solutions to the challenges faced by protamine sulfate. These efforts include the development of protamine-like compounds (Byun, Y. et al. Low Molecular Weight Protamine: A Potential Nontoxic Heparin Antagonist. Thromb. Res. 1999, 94 (1), 53-61.), functionalized protamine derivatives (Kaminski, K et al. Cationic Derivatives of Dextran and Hydroxypropylcellulose as Novel Potential Heparin Antagonists. J. Med. Chem. 2011, 54 (19), 6586-6596.), and peptide-based approaches (Liu, Q. et al. Serum Albumin-Peptide Conjugates for Simultaneous Heparin Binding and Detection. ACS Omega 2019, 4 (26), 21891-21899.). While low molecular weight protamine (LMWP) with fragment sizes of approximately 1500 Da, maintain heparin neutralization properties, the pharmacological and toxicological profiles of this heparin antidote are unresolved. (He, H. et al. Low Molecular Weight Protamine (LMWP): A Nontoxic Protamine Substitute and an Effective Cell-Penetrating Peptide. J. Controlled Release 2014, 193, 63-73.). As alternatives to analogs of conventional protamine-based antidotes, cationic polymers provide heparin reversal with preferred properties and function. Because cationic polymers are synthetic, it is possible to control the final structure of the drug candidates while ensuring purity of the final materials. Improved control results in predictable pharmacokinetic profiles and removes risks that arise from compounds derived from biological sources. Recent examples of synthetically derived cationic polymers have been shown to be effective in vitro in the reversal of select heparins (Kalaska, ibid, Kaminski, ibid, and Kalaska, B. et al. Nonclinical Evaluation of Novel Cationically Modified Polysaccharide Antidotes for Unfractionated Heparin. PLOS ONE 2015, 10 (3), e0119486.), at the expense of abnormal bleeding, cell apoptosis, cell death, and histopathological changes including renal abnormalities and lung congestion due to the high density of cationic charges present. (Jones, ibid, Sokolowska, E. et al. The Toxicokinetic Profile of Dex40-GTMAC3-a Novel Polysaccharide Candidate for Reversal of Unfractionated Heparin. Front. Pharmacol. 2016, 7, 60., and Labieniec-Watala, M. and Watala, C. PAMAM Dendrimers: Destined for Success or Doomed to Fail? Plain and Modified PAMAM Dendrimers in the Context of Biomedical Applications. J. Pharm. Sci. 2015, 104 (1), 2-14.).

In experiments conducted in the development of the present invention, MPIs were tested as safer and more effective heparin reversal agents. The unique combination of cationic binding groups on a biocompatible scaffold enables a selective change in protonation state upon MPI binding to targeted anionic binding partners. The protonation properties and selective protonation properties of MPIs have been described. A change in overall charge upon polyanion binding provides selective binding and overcomes energetic barriers resulting in increased efficiency. In experiments conducted in the development of the present invention, we developed novel heparin antidotes by generating and screening a library of MPI molecules. While switchable protonation is present in most MPI candidates studied, MPI 2 generates multiple preferred activities including potent universal heparin neutralization, hemocompatibility, and no effect on bleeding, standing as an improved, useful and safe heparin antidote compared previously reported compounds. (Shenoi, ibid, Travers, ibid, and Kalathottukaren and Abraham, ibid.)

Using serial assays, MPI 2 was identified as an attractive agent from the library of inhibitors. MPI 2 is composed of a 23 kDa HPG-mPEG scaffold conjugated with ˜24 CBG I group per molecule resulting in an average charge of 35 positive charge at physiological pH. In comparison to UHRA, the charge of MPI 2 is significantly lower at physiological pH. Use of linear aliphatic triamine CBG-I ligand unlike Me₆TREN in UHRA, reduces the charge state at physiological pH, resulting in a lower average number of charges per MPI. As well, the linear ligands demonstrate switchable protonation upon polyanion (e.g., heparin) binding to enhance the heparin reversal properties of MPIs derived from linear aliphatic CBGs. Accordingly, not all cationic residues on previous UHRA are necessary to stabilize binding with heparin because MPI 2 provides more effective heparin reversal using a ligand that exhibits a significant reduction in average cationic charge both per ligand and per macromolecule.

The reduction in net cationic charge on MPI 2 with increased selectivity to heparins provides multiple advantages over conventional heparin antidotes. For example, MPI 2 reverses multiple types of heparins including UFH, LMWH and fondaparinux with high efficiency whereas protamine sulfate (PS), the only FDA-approved heparin antidote, exhibits minimal reversal of LMWH anticoagulant activity, and is unable to reverse the effects of fondaparinux. (Mahan, C. E. A 1-Year Drug Utilization Evaluation of Protamine in Hospitalized Patients to Identify Possible Future Roles of Heparin and Low Molecular Weight Heparin Reversal Agents. J. Thromb. Thrombolysis 2014, 37 (3), 271-278.). Moreover, besides being ineffective as an antidote to all LMWHs and fondaparinux, PS has other undesirable properties when used as a heparin reversal therapeutic. (Pevni, D. et a. Protamine Induces Vasorelaxation of Human Internal Thoracic Artery by Endothelial NO-Synthase Pathway. Ann. Thorac. Surg. 2000, 70 (6), 2050-2053., Pretorius, M. et al, Pilot Study Indicating That Bradykinin B2receptor Antagonism Attenuates Protamine-Related Hypotension after Cardiopulmonary Bypass. Clin. Pharmacol. Ther. 2005, 78 (5), 477-485., Jenkins, C. S. P. et al. Interactions of Polylysine With Platelets. Blood 1971, 37 (4), 395., Bakchoul, T et al. Anti-Protamine-Heparin Antibodies: Incidence, Clinical Relevance, and Pathogenesis. Blood 2013, 121 (15), 2821., and Fairman, R. Pet al. Protamine Sulfate Causes Pulmonary Hypertension and Edema in Isolated Rat Lungs. J. Appl. Physiol. 1987, 62 (4), 1363-1367.). While protamine is an effective antidote to UFH (Garcia, D. et al. Parenteral Anticoagulants: Antithrombotic Therapy and Prevention of Thrombosis, 9th Ed: American College of Chest Physicians Evidence-Based Clinical Practice Guidelines. Chest 2012, 141 (2 Suppl), e24S-e43S.), it must be carefully titrated upon administration to prevent serious adverse side effects that occur on overdose. As shown in FIG. 33a, the therapeutic window of PS is very narrow when used to treat UFH based on observed clot time in an aPTT assay with heparinized plasma. MPI 2 however, provides full reversal of UFH over a wide range of concentrations, and is a significant improvement over PS and over UHRA. While an understanding of the mechanism is not necessary to practice the invention, and the present invention is not limited to any particular mechanism, the minimized quantity of charge on MPI 2 may be responsible for attenuation of the complications observed for PS and other cationic polymers (Sokolowsda, ibid.) In turn, MPI 2 outperforms UHRA and PS in the absence of heparin. Unlike most cationic compounds that interact with proteins in blood and interfere with hemostasis (Jones, ibid, Hu, ibid, Boer, C. et al Anticoagulant and Side-Effects of Protamine in Cardiac Surgery: A Narrative Review. Br. J. Anaesth. 2018, 120 (5), 914-927., and Törnudd, M. et al. Protamine Stimulates Platelet Aggregation in Vitro with Activation of the Fibrinogen Receptor and Alpha-Granule Release, but Impairs Secondary Activation via ADP and Thrombin Receptors. Platelets 2020, 0 (0), 1-7.). MPI 2 exhibits no sign of activation of platelets and does not interfere with any parameters of whole blood clotting. Thromboelastometry (ROTEM) is commonly used in clinical settings to assess the effects of anticoagulants in patient samples and guide the administration of reversal agents to mitigate excess bleeding (Bolliger, D. et al. Principles and Practice of Thromboelastography in Clinical Coagulation Management and Transfusion Practice. Transfus. Med. Rev. 2012, 26 (1), 1-13., and Wang, S.-C et al. Thromboelastography-Guided Transfusion Decreases Intraoperative Blood Transfusion During Orthotopic Liver Transplantation: Randomized Clinical Trial. Transplant. Proc. 2010, 42 (7), 2590-2593.). Using ROTEM, we demonstrate that MPI 2 fully reverses UFH in whole blood while having no measurable effects on clotting at 5 times the concentrations needed for UFH reversal. Such versatility is demonstrated not only by the clot time and time to clot formation in ROTEM, but also by the maximum clot firmness and the overall clot trace, a strong indicator of the quality and stability of the overall clot formed (Shenkman, ibid.) These results support the many advantages of minimizing the positive charges on the MPI antidote, with no decrease in heparin sensitivity. As such, MPI 2 provides optimal characteristics of a heparin reversal agent in vitro. A further advantage is that switchable protonation is an important tool in the design of highly biocompatible polycations. Conventional polycations are notorious for their poor blood compatibility and toxicity (Kim, ibid, La, ibid, Moreau, ibid.)

The advantages of MPI 2 are further highlighted using mouse tail bleeding models in vivo to establish the effects of anticoagulants and reversal of anticoagulants (Monroe, D. M. and Hoffman, M. A Mouse Bleeding Model to Study Oral Anticoagulants. Proc. 7th Symp. Hemost. Old Syst. New Play. New Dir. May 15-17 2014 Chap. Hill N. C. USA 2014, 133, S6-S8., Getz, T. M. et al. Mouse Hemostasis Model for Real-Time Determination of Bleeding Time and Hemostatic Plug Composition. J. Thromb. Haemost. 2015, 13 (3), 417-425., and Vaezzadeh, N. et al. Comparison of the Effect of Coagulation and Platelet Function Impairments on Various Mouse Bleeding Models. Thromb Haemost 2017, 112 (08), 412-418.). MPI 2 fully reverses the effects of 200 U/kg UFH and 200 U/kg enoxaparin in mice. When mice are administered MPI 2 without heparins, it has no significant effects on mice bleeding times or hemoglobin loss even at concentrations significantly higher than effective doses, thereby demonstrating that MPI 2 does not interfere with normal hemostasis. These observations provide support for the therapeutic role of MPI 2as a heparin reversal agent. Other heparin antidotes cannot provide such a large therapeutic window and may require careful titration upon administration (Kalathottukaren and Abraham, ibid, and Boer, ibid). The extended therapeutic window and minimal bleeding side effects with MPI 2 administration are particularly notable when compared to UHRA. While previous generation UHRAs demonstrated biocompatibility and potency (Shenoi, ibid, Kalathottukaren and Abraham, ibid), MPI 2 provides a substantially large therapeutic window for heparin neutralization for all heparins.

Compared to other PS alternatives, MPI 2 presents multiple advantages over protamine variants such as delparantag (McAllister R. Abstract 17322: Heparin-Antagonist PMX-60056 Rapidly and Completely Reverses Heparin Anticoagulation in Man. Circulation 2010, 122 (suppl_21), A17322-A17322), and PM102. (Shenoy, S. et al. Development of Heparin Antagonists with Focused Biological Activity. Curr. Pharm. Des. 1999, 5 (12), 965-986., and Cushing, D. J. et al. Reversal of Heparin-Induced Increases in APTT in the Rat by PM102, a Novel Heparin Antagonist. Eur. J. Pharmacol. 2010, 635 (1), 165-170.) Advantages over these peptide-based approaches includes the reduction of contamination that is common to biologically derived therapeutics. Our fully synthetic methods eliminate this risk at a low cost while simultaneously ensuring control of the final structure of the reversal agent. Other cationic polymers for electrostatic binding to heparins have been investigated, such as a pegylated cationic poly(3-(methacryloylamino)propyl)trimethylammonium chloride (PMAPTAC) block polymers prepared by Kalaska and colleagues. (Kalasaka, ibid.) While their heparin binding copolymer (HBC) showed reversal of low concentrations (on the range of 3-10 mg/kg) of enoxaparin and fondaparinux in vivo, the safety of HBC with bleeding at higher concentrations than necessary to reverse heparin was not reported. As seen by administration of high concentrations of other cationic structures such as PS, extended bleeding times and other complications, such as hypotension, can be lethal in some cases. (Labieniec-Watala, ibid, Boer, ibid.) MPI 2 does not extend bleeding times or hemoglobin loss at high concentrations, and its high biocompatibility underscores the utility of MPI 2 as a heparin antidote, thereby avoiding the complications when administering appropriate doses of PS or other potential protamine alternatives.

The present invention provides a new series of macromolecular polyanion inhibitors (MPIs) generated using cationic binding groups composed of linear alkyl amines. From this library of cationic polymers, compounds have been identified as safe and specific heparin antidotes. Candidate MPIs were screened by aPTT and calibrated automated thrombography, that highlight potent heparin reversal activity of MPI 2 over a broad range of concentrations tested. Heparin reversal activity of MPI 2 demonstrates a broad therapeutic window. Further characterization via thromboelastometry shows enhanced heparin reversal by MPI 2 vs. UHRA. Hemocompatibility of the MPIs from reduced net cationic charge per molecule provides optimized heparin reversal agents. Studies in vivo using a mouse tail bleeding model further confirm potent heparin reversal activity of MPI 2, and minimal bleeding side effects the polymeric therapeutics, even at doses an order of magnitude above the therapeutic dose. Together, these results show enhanced heparin reversal activity and minimized non-specific interactions achieved via the development of macromolecular polyanion inhibitors using a combination of tunable protonation properties of ligands, and highly biocompatible polymer scaffold towards novel therapeutics.

### Protonation of Cationic Ligands in the Design of Safe and Effective Nucleic Acid Inhibitors

We determined the activities of MPIs as nucleic acid inhibitors. Diverse inhibitory compounds were used with PolyIC (Polyinosinic:polycytidylic acid) as a nucleic acid for the screening studies. MPI 3 was shown to correct the thrombotic and antifibrinolytic effect of nucleic acids in blood plasma.

### Pharmaceutical Compositions and Formulations

The present disclosure further provides pharmaceutical compositions (e.g., comprising the compounds described above and elsewhere herein). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral, intravenous or parenteral. Parenteral administration includes intravenous, intra-arterial, subcutaneous, intraperitoneal or intramuscular injection or infusion; or intracranial, e.g., intrathecal or intraventricular, administration. Administration may be achieved by single shot, a series of single shots, and/or by continuous administration. In certain embodiments, continuous administration is provided by a programmable external pump. In other embodiments, continuous administration is provided by a programmable implantable pump.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is affected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual compounds, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### EXPERIMENTAL EXAMPLES

The following examples are provided to demonstrate and further illustrate certain embodiments and aspects of the present disclosure and are not to be construed as limiting the scope thereof.

### Materials and Methods

All chemicals were purchased and used without further purification, unless indicated otherwise. Glycidol was purified by vacuum distillation and stored at 4° C. using molecular sieves (5 Å). Cellulose ester dialysis membranes were obtained from Spectra/Por Biotech (Rancho Dominguez, CA, USA). CDCl₃ or D₂O (Cambridge Isotope Laboratories, Andover MA) were used as solvents, with the relevant solvent peak as reference. Chelating resin (Chelex® 100) was purchased from Bio-Rad. Human fibrinogen (Fibrinogen), polyethyleneimine (PEI, 25 kDa), thrombin, protamine sulphate and N-2-hydroxyethyl piperazine-N′-2-ethanesulfonic acid (HEPES) were purchased from Sigma Aldrich. Recombinant tissue factor (TF, Innovin) was purchased from Dade-Behring. Polystyrene 96-well microplates (Costar) used for clotting assays were purchased from Corning. A microplate reader from Spectramax was used. Reagents used for calibrated automated thrombography such as thrombin calibrator, Flu-Ca solution and Immulon microplates were purchased from Diagnostica Stago. Citrated pooled normal human plasma (PNP) from 20 donors was purchased from Affinity Biologicals (ON, Canada). Buffer for biological assays was prepared with 20 mM HEPES with 150 mM NaCl, pH 7.4 unless otherwise stated. BD Vacutainer® Citrate Tubes containing 3.2% buffered sodium citrate solution were purchased from Becton, Dickinson and Company (New Jersey, USA). All other chemicals were purchased and used without further purification, unless indicated otherwise. Blood was drawn from consenting informed healthy volunteer donors at Centre for Blood Research, University of British Columbia, in vials containing EDTA or sodium citrate.

### Techniques

A NE-1000 Programmable Single Syringe Pump (Farmingdale, NY) was used for chemical synthesis and polymerization. Absolute molecular weights of the polymers were determined by GPC on a Waters 2695 separation module fitted with a DAWN EOS multi-angle laser light scattering (MALLS) detector coupled with Optilab DSP refractive index detector from Wyatt Technology. GPC analysis was performed using Waters ultrahydrogel 7.8×300 columns (guard, 250 and 120) and 0.1 N NaNO₃ at pH 7.4 using 10 mM phosphate buffer as the mobile phase. ¹H NMR spectra were recorded on a Bruker Advance 300 MHz NMR spectrometer and Bruker Advance 400 MHz NMR spectrometer.

### Polymer Synthesis, Modification and Conjugation

mPEG₃₅₀ Epoxide

To a 500 mL round bottom flask was added mPEG₃₅₀ (110 g, 314 mmol, 1.0 eq.) with magnetic stir bar. To this, crushed NaOH pellets (39 g, 940 mmol, 3.0 eq.) were added and allowed to stir for 24 hours. The reaction mixture was then cooled to 0° C. and epichlorohydrin (53 mL, 658 mmol, 2.0 eq.) was slowly added over the course of 3 hours. The reaction was kept cool by replenishing the ice bath over the course of the slow addition. After complete addition of epichlorohydrin, the reaction mixture was stirred for an additional 24 hours and slowly warmed to room temperature. Once complete, the reaction was quenched with MeOH, and the remaining salts filtered off with DCM. Crude product was concentrated under vacuum. The crude product was dried on a vacuum drying line for 2 days to evaporate unreacted epichlorohydrin, affording the pure mPEG₃₅₀ epoxide in 90% yield.

¹H NMR (300 MHz, Chloroform-d): δ 3.78 (dd, J=11.7, 3.1 Hz, 1H), 3.73-3.51 (m, 33H), 3.47-3.35 (m, 4H), 3.16 (dq, J=6.1, 3.1 Hz, 1H), 2.79 (t, J=4.6 Hz, 1H), 2.61 (dd, J=5.0, 2.7 Hz, 1H).

### HPG-PEG Synthesis

A 3-neck round bottom flask was cooled under vacuum and filled with argon. To this, 1,1,1-tris(hydroxymethyl)propane (TMP, 167 mg) and potassium methylate (25 wt % solution in methanol, 0.110 mL) were added and stirred for 30 minutes. Methanol was removed under high vacuum for 4 hours. The flask was heated to 95° C. and distilled glycidol (3.8 mL) was added over a period of 15 hours. After complete addition of glycidol, the reaction mixture was stirred for an additional 12 hours. Then, mPEG₃₅₀-epoxide (10.5 mL) was added over a period of 12 hours at 95° C. The reaction mixture was stirred for additional 4 hours. The reaction was cooled to room temperature, quenched with methanol, then passed through Amberlite IR-120H resin to remove the potassium ions and twice precipitated from diethyl ether. The polymer was then dissolved in water and dialyzed in water membrane for 3 days with periodic changes in water.

¹H NMR (300 MHz, Chloroform-d) δ 4.04-3.40 (m, 50H), 3.38 (s, 3H).

mPEG content (by ¹H NMR): 25 mol %; Polyglycerol: 75 mol %.

GPC-MALLS (0.1 M NaNO₃): Mₙ 24 000; Mw/Mₙ 1.3.

For 10 kDa polymers, the same synthetic procedure was followed with higher initiator:monomer ratio. A 3-neck round bottom flask was cooled under vacuum and filled with argon. To this, 1,1,1-tris(hydroxymethyl)propane (TMP, 268 mg) and potassium methylate (25 wt % solution in methanol, 0.178 mL) were added and stirred for 30 minutes. Methanol was removed under high vacuum for 4 hours. The flask was heated to 95° C. and distilled glycidol (5.71 mL) was added over a period of 23 hours. After complete addition of glycidol, the reaction mixture was stirred for an additional 3 hours. Then, mPEG₃₅₀-epoxide (12.8 mL) was added over a period of 12 hours at 95° C. The reaction mixture was stirred for additional 4 hours. The reaction was cooled to room temperature, quenched with methanol, then passed through Amberlite IR-120H resin to remove the potassium ions and twice precipitated from diethyl ether. The polymer was then dissolved in water and dialyzed in water membrane for 3 days with periodic changes in water.

¹H NMR (300 MHz, Chloroform-d) δ 4.04-3.40 (m, 50H), 3.38 (s, 3H).

mPEG content (by ¹H NMR): 27.5 mol %; Polyglycerol: 72.5 mol %.

GPC-MALLS (0.1 M NaNO₃): Mₙ 10 360; Mw/Mₙ 1.2.

### HPG-mPEG-OTs

The reaction was carried out under an inert gas atmosphere and exclusion of water. (Roller, S. et al. High-Loading Polyglycerol Supported Reagents for Mitsunobu- and Acylation-Reactions and Other Useful Polyglycerol Derivatives. Mol. Divers. 2005, 9 (4), 305-316.) HPG-mPEG (200 mg) in a 3-necked 100 mL flask with thermometer magnetic stirrer was dissolved in pyridine (5 mL). The solution was cooled to 0° C. by an ice/NaCl bath, then, and a solution of tosyl chloride (TsCl) (1.2 eq. of target average number of groups) in pyridine (5 mL) was added dropwise so that the temperature did not exceed 5° C. The brown mixture was stirred for 16 hours as the reaction was allowed to warm to room temperature. Then solvent was removed in vacuo. The resulting mixture was then dialysed in water for 3 days with periodic changes in water to give a honey-like product.

¹H NMR (400 MHz, Chloroform-d): δ 7.78 (m, 0.7H), 7.34 (m, 0.7H), 3.98-3.39 (m, 48H), 3.35 (s, 3H), 2.45 (s, 1.05H).

### Hpg-Mpeg-Nh₂

In a 50 mL 1-necked flask with reflux condenser and magnetic stirrer, HPG-mPEG-OTs (200 mg, 0.20 mmol OTs-groups) was dissolved in anhydrous 1,4-dioxane (10 mL). (Roller, ibid.) After addition of CBG (1 mmol, 5.0 eq.), the resulting suspension was heated to reflux at 115° C. for 24 hours. After cooling the reaction mixture, the mixture was dialyzed in water for 3 days with periodic changes in water to give a brown honey-like product.

¹H NMR (300 MHz, Chloroform-d): δ 3.89-3.43 (m, 35H), 3.38 (s, 3H), 2.87 (m, 9H).

### HPG-mPEG-NMe₂

Amine functionalized HPG-mPEG (200 mg, 24 CBG per polymer), was transferred to a 20 mL side-necked flask. The reaction mixture was dissolved in DI water at room temperature. 10 mL of a formic acid/formaldehyde (1:1) mixture was added dropwise to the reaction flask. The reaction was stirred and heated to reflux at 110° C. for 24 hours. After cooling the reaction mixture, the mixture was dialyzed in water for 22 days with periodic changes in water until the pH of the water was neutral. Upon lyophilizing to remove excess water, CBG linked polymer was collected.

¹H NMR (300 MHz, Chloroform-d): δ 8.11 (s), 4.11-3.19 (m), 3.19-2.95 (m), 2.78-1.94 (m).

### Physical Characterizations

### Conductometric Measurements.

Time allowed for equilibration was 15 seconds for conductometry titrations. A solution of CBG in water (0.1 mM) was acidified dropwise with 1.0 M HCl and titrated with carbonate-free NaOH (0.5012 M) that was standardized against freshly recrystallized potassium hydrogen phthalate. Temperature was kept constant at 25° C. with a warm water bath. Titration curves were manually fitted to calculate ¹H concentration.

### Potentiometric Measurements

All data were collected in triplicate. Prior to each titration, the electrode was calibrated using a standard HCl solution. Calibration data were calculated to obtain parameters E₀ and pKₐ. Time allowed for equilibration was 10 minutes for pKₐ titrations. Solutions were titrated with carbonate-free NaOH (0.141 M) that was standardized against freshly recrystallized potassium hydrogen phthalate. Protonation equilibria of the CBG were investigated by NaOH titrations of a solution containing (CBG) 1.1×10⁻³ M at 25° C. and 0.16 M NaCl ionic strength. Potentiometric data were processed using HyperQuad2013 software. (Gans, P. et al. Investigation of Equilibria in Solution. Determination of Equilibrium Constants with the HYPERQUAD Suite of Programs. Talanta 1996, 43 (10), 1739-1753.) Titrations were performed on a 809 Titrando from Metrohm. Temperature was kept constant with a circulating water bath.

### Surface Plasmon Resonance

The interaction of MPI and surface-bound polyP was carried out at 25° C. using a T200 Biacore (GE Healthcare, Life Sciences). Samples were prepared by making stock solutions of at least 100× the desired concentration at high volume and filtered using 0.2 m filters (Millex, Merck Millipore Ltd). Running buffer was prepared with 20 mM HEPES with 100 mM NaCl, 1 mM EDTA and 0.005% P20 surfactant. Running buffer was degassed, filtered and the pH was 7.4. Prior to titration, stock solutions of MPI were serially diluted with same running buffer. Multi-cycle assays were run with 1 M NaCl injection after each cycle. Using a flow rate of 30 L/min, an injection volume of 60 μL and therefore an injection over 120 seconds, a dissociation was run over 60 seconds. 7-8 concentrations for each MPI were run for each steady state affinity plot. All samples were run on a Series S sensor chip CM4 (GE Healthcare, Life Sciences), with streptavidin bound to the surface to allow for capture of biotinylated polyP. Three of the 4 flow cells contained biotinylated long chain polyP (P1075), biotinylated medium chain polyP (P560), and biotinylated platelet sized polyP (P110), while one flow cell contained streptavidin but no polyP (the reference cell). Data was analyzed on the Biacore T200 Software (GE Healthcare, Life Sciences).

### Isothermal Titration Calorimetry

The interaction of MPI and polyP was carried out at 25° C. using a MicroCal iTC200 calorimeter (MicroCal, Northampton, USA). Samples were prepared by making stock solutions of at least 100× the desired concentration at high volume and filtered using 0.2 m filters (Millex, Merck Millipore Ltd). The pH of each solution was then measured to ensure that they were between 7.35 and 7.40. Prior to titration, stock solutions were diluted with the same filtered buffer and degassed. Titrations consisted of 25 consecutive injections of 1.5 μL volume and 5 sec duration each, with a 3 min interval between injections with only the first injection between 0.2 μL volume, not used as part of the fit. Heat of dilution was measured by injecting MPI solution into buffer alone and was subtracted from the experimental curves prior to data analysis. The resulting data were fit into a single set of identical sites model using the MicroCal ORIGIN software supplied with the instrument.

### P Nuclear Magnetic Resonance Spectrometry

NMR spectra for MPI and polyP binding interaction studies were acquired on a Bruker 500 MHz instrument (Bruker Biospin, Milton, ON), operating at a ¹H frequency of 499.4 MHz. ³¹P NMR spectra were collected at 298 K. The NMR sample was prepared to yield 0.5 mM of polyphosphate in HEPES buffer with 150 mM NaCl and 10% D₂O (by volume) for a total sample volume of 600 μL. All polyphosphate concentrations are provided in terms of the concentration of phosphate monomer. For the binding study, polyphosphate was first prepared with a known concentration of internal standard of trimethylphosphate. MPI was titrated in, and the total volume was increased by 1.5 μL of the 600 μL total per addition.

### In Vitro Studies

### Plasma Clot Formation by Turbidity Analysis in a TF-Triggered System

Microplate turbidimetric clotting assays were performed with platelet-poor plasma (PPP) obtained from three donors. MPI solutions were prepared in 20 mM HEPES (pH 7.4, 150 mM NaCl) buffer. Clotting was initiated in 90 μL of 30% diluted PPP spiked with MPI (dilution 1:10) by adding 5 μL of recombinant tissue factor (TF; Innovin (1:10,000; 0.73 μM)) and 5 μL of CaCl₂) (20 mM). Clotting was evaluated by monitoring changes in turbidity (A405 nm) every 30 seconds with the Spectramax microplate reader for 2 hours at 37° C. Clotting parameters including lag time were calculated and considered as the time point when an exponential increase in absorbance was first observed.

### Platelet Activation Analysis by Flow Cytometry

Platelet activation was quantified by flow cytometry. Ninety microliters of platelet rich plasma (PRP) were incubated at 37° C. with 10 μL of stock MPI samples for one hour. 10 μL of post-incubation platelet/polymer mixture was diluted in 45 μL PPP and incubated for 15 minutes in the dark with 5 μL of monoclonal anti-CD62-PE (Immunotech, Marseille, France). The reaction was then stopped with 0.5 mL of phosphate-buffered saline solution. The level of platelet activation was analyzed in a BD FACS Canto II flow cytometer (Becton Dickinson, ON, Canada) by gating platelet-specific events based on their light scattering profile. Activation of platelets was expressed as the percentage of platelet activation marker CD62P-PE (phycoerythrin) fluorescence detected in the 10,000 total events counted. Triplicate measurements were performed and the mean was recorded. Thrombin receptor activator receptor 6 (TRAP6), a recognized platelet activator (SigmaAldrich, Oakville, ON, Canada), was used as a positive control for the flow cytometric analysis.

Determination of polyP Inhibition Activity by Serine Protease Activity Assay

Serine protease assays were carried out at 37° C. by measuring the absorbance intensity upon cleavage of a chromogenic substrate, Chromogenix S-2288 that is sensitive to a broad spectrum of serine proteases. Commercially available pooled platelet poor plasma (PPP, 20 donors) was purchased from Affinity Biologicals. A stock solution of MPI was prepared at concentrations for a 1:10 dilution of the polymer solution. Coming 96 well plates were pre-treated with 3% BSA solution for 30 minutes at room temperature and subsequently washed 3× with tricine buffer (10 mM tricine+150 mM NaCl).

For the inhibition studies, MPI were incubated with polyP-substrate-buffer mixture for 30 minutes at 37° C. In this mixture, polyphosphates of length 700 monomer units per polymer (P700) were used at a final concentration of 100 μM monomer concentration. A Final substrate concentration of 200 μM was prepared from a working stock after reconstitution following manufacturer's instructions. As a negative control, a solution of tricine buffer mixed with MPI was incubated without addition of P700, making up the solution difference with tricine buffer. As a positive control, P700 was incubated with buffer without addition of pPBA. Buffer control was also recorded, containing tricine buffer and chromogenic substrate only. 120 μL of the mixtures were pipetted out into a 96 well plate in triplicates. The serine protease assay was initiated by addition of 80 μL of the chromogenic substrate-polyP-pPBA mixture to each well containing 20 μL of pre-warmed (37° C.) plasma with a multichannel pipette, making the total reaction volume 100 μL. Serine protease activity was measured and recorded as absorbance intensity over time. The absorbance intensity was recorded at 37° C. every 15 seconds over a period of 45 minutes on a SpectraMax M3 plate reader at an excitation wavelength of 405 nm. Based on the positive control, a time frame of one minute was selected for analysis where the activity presented in mOD would be plotted over the one minute for each well, to obtain the activity of serine protease.

### Thrombin Generation Assay by Calibrated Automated Thrombography for the Evaluation of MPI Inhibition Activity

A thrombin generation assay was carried out at 37° C. by measuring the fluorescence intensity upon cleavage of the fluorogenic substrate Z-Gly-Gly-Arg-AMC by regenerated thrombin. Commercially available pooled normal platelet poor plasma (PNP, 30 donors) from George King Bio-Medical, USA was mixed 1:1 with HBS (20 mM HEPES with 100 mM NaCl at pH 7.4). Phosphatidylcholine (80): phosphatidylserine (20) (PCPS) liposomes were added to obtain a final concentration of 20 μM. Serial dilutions of MPI candidates and UHRA were prepared fresh for these experiments. Experiments were repeated twice with two technical replicates each. Thrombin calibrator was used following the manufacturer's instructions, and the thrombin generation assay was initiated by the addition of fluorogenic substrate (both from Diagnostica Stago). Substrate hydrolysis was monitored on a fluorescent plate reader from Diagnostica Stago. The fluorescence intensity was recorded at 37° C. every 30 seconds over a period of 1.5 hours and analyzed using Thrombinoscope™ software from Diagnostica Stago.

### Determination of the Effect of MPI on Thrombin Generation in a TF Triggered Plasma System

In order to determine what affect each MPI candidate (Error! Reference source not found.) had on thrombin generation in the absence of polyP, TF (Thrombinoscope™) was added to initiate clotting at a final concentration of 5 μM. Final concentrations of MPIs ranged from 5-50 μg/mL. No other clotting initiators or accelerators were added.

TABLE 1
Structures of representative amine structures analyzed for protonation
state properties in the design of CBGs

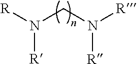

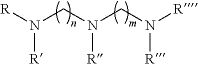

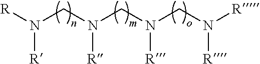

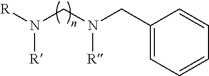

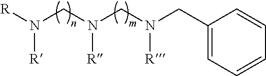

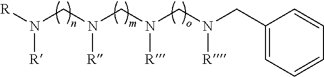

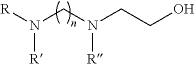

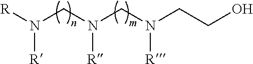

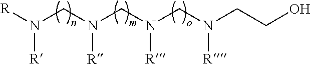

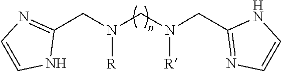

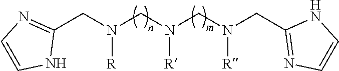

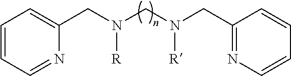

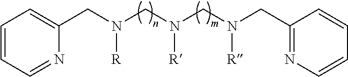

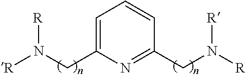

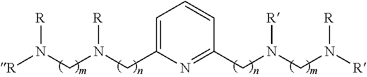

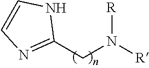

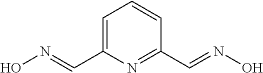 [SMILES: O/N=C/c1cccc(/C=N/O)n1]

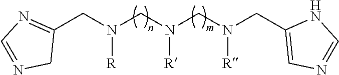

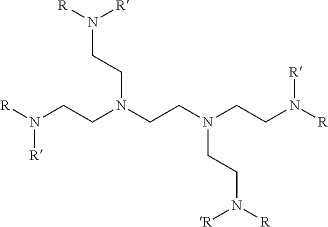

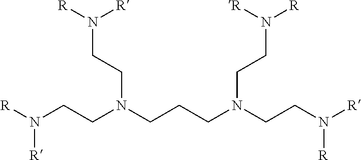

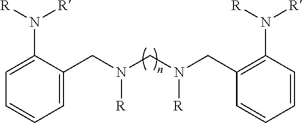

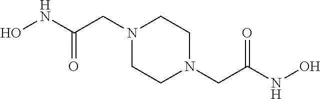 [SMILES: O=C(CN1CCN(CC(=O)NO)CC1)NO]

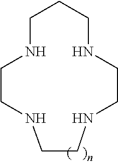

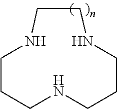

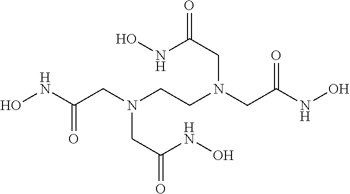 [SMILES: O=C(CN(CCN(CC(=O)NO)CC(=O)NO)CC(=O)NO)NO]

R = H, CH₃, CH₂CH₃
R′ = H, CH₃, CH₂CH₃
R″ = H, CH₃, CH₂CH₃
R′′′ = H, CH₃, CH₂CH₃
n = 1, 2, 3, 4
m = 1, 2, 3, 4

Determination of Long Chain Polyphosphate (LC polyP) Inhibition by Thrombin Generation Assay

To determine the efficacy of LC polyP inhibition, a mixture of plasma, PCPS and MPI or UHRA were incubated with LC polyP (200 μM) for 3 minutes at 37° C. prior to the initiation of clotting. Concentrations of inhibitors tested ranged from 0.2-100 μg/mL.

Determination of Short Chain Polyphosphate (SC polyP) Inhibition by Thrombin Generation Assay

To determine the efficacy of SC polyP inhibition, FXII deficient plasma (Haemtech) was used. To a mixture of plasma, PCPS and MPI, SC polyP was added at a final concentration of 5 μM. TF (Thrombinoscope™) at a final concentration of 8.3 fM was also included in the mixture. The inhibitor concentrations tested ranged from 0.2-100 μg/mL.

### Effect on Plasma Clotting in FXII Deficient Plasma

For the clotting assay, each well was filled with 100 μL of a mixture containing FXII deficient plasma (Haemtech) (50 μL), MPI (100 μg/mL, final) in HEPES buffered saline with bovine serum albumin (HBSA, 20 mM HEPES and 100 mM NaCl at pH 7.4 with 0.1% BSA) and relipidated tissue factor in 30% PCPS liposomes. This mixture was incubated for 120 seconds at 37° C., then clotting was initiated by addition of 50 μL pre-warmed (37° C.) 25 mM CaCl₂). Clot time was measured on a STart 4® coagulometer (Diagnostica Stago, France).

Determination of polyP Inhibition Activity by Viscosity-Based Plasma Clotting Assay

MPI solutions were prepared in 10 mM tricine buffer (pH 7.4, 50 μM ZnCl₂ and 150 mM NaCl). For inhibition studies, citrated human PPP from Affinity Biologicals (20 donors, pooled) was warmed to 37° C. and incubated with MPI solutions and polyP (700 monomer units at 20 μM final monomer concentration) at 37° C. for 15 minutes such that the final plasma concentration consisted of 50% of the reaction mixture. The final concentration of MPI in plasma ranged from 2.5 to 100 μg/mL. As a positive control, plasma was incubated with MPI without addition of polyP, maintaining the plasma concentration at 50%. As a negative control, plasma was incubated with tricine buffer, again maintaining the same concentration of plasma. Clotting was initiated by addition of a clotting mixture comprised of recombinant tissue factor (Dade® Innovin®rTF, Siemens/Dade-Behring), an 80:20 PCPS mixture and CaCl₂) at final concentrations of 0.24 μM, 25 μM and 7.7 mM, respectively. One hundred microliters of the plasma mixture were transferred to cuvette-strips at 37° C. and clotting was initiated with addition of 50 μL of the clotting mixture. The clotting time was measured on a STart 4® coagulometer (Diagnostica Stago, France). Because each experimental cuvette strip had 4 wells, each experiment was run with one negative and one positive controls. All experiments were performed in triplicate and the average values (mean±standard error of the mean) are reported.

### Platelet Activation Analysis by Flow Cytometry

The level of platelet activation was quantified by flow cytometry. Ninety microliters of PRP were incubated at 37° C. with 10 μL of stock MPI samples (MPI 1, MPI 6 and MPI 8 at final concentrations ranging from 50-200 μg/mL) for 1 hour. Ten microliters of post-incubation platelet/MPI mixture were diluted in 45 μL PPP from the same donor and incubated for 15 minutes in the dark with 5 μL of monoclonal anti-CD62-PE (Immunotech, Marseille, France). The reaction was then stopped with 0.5 mL of phosphate-buffered saline solution. The level of platelet activation was analyzed from flow cytometry profiles by gating platelet-specific events based on their light scattering profile. Flow cytometry profiles were acquired using a 3-laser CytoFLEX flow cytometer from Beckman Coulter Life Sciences (Indianapolis, IN). Activation of platelets was expressed as the percentage of platelet activation marker CD62P-PE (phycoerythrin) fluorescence detected in the 10,000 total events counted. Triplicate measurements were done, the mean of which was recorded. Thrombin receptor activator peptide 6 (TRAP 6), a recognized platelet activator, (Sigma Aldrich, Oakville, ON, Canada) was used as a positive control for platelet activation analysis.

### Influence of MPI on Whole Blood Clotting by Thromboelastometry

Whole blood was mixed with MPI (MPI 1, MPI 6 and MPI 8 with a final concentration of 100 μg/mL) in HEPES buffered saline (HBS) (20 mM HEPES+150 mM NaCl), and analyzed for whole blood clotting using a ROTEM® delta from Tem Innovations GmbH (Instrumentation Laboratory as of 2016) at 37° C. Stock solutions of the MPIs and UHRA were prepared at concentrations 100× the final desired concentration in HBS. Citrate anticoagulated whole blood (356 μL) was mixed with 44 μL of the MPIs or UHRA. Three hundred and forty microliters of this suspension were transferred into the ROTEM cup and was re-calcified with 20 μL of 0.2 M calcium chloride solution. HBS mixed with whole blood was used as a negative control for the experiment.

### Fibrin Clot Structure and Fiber Diameter by Scanning Electron Microscopy (SEM)

The effects of MPI 8 on the overall fibrin clot structure and fibrin diameter of the clot in the presence of MPI 8 were assessed by SEM. All samples were randomly coded and blinded to the individual performing the imaging analysis to avoid bias. Fibrin clots were prepared in sterile, 5 mL round-bottom polypropylene tubes (BD Falcon) by mixing human fibrinogen (2.6 mg/mL) in 20 mM HEPES (pH 7.4 and 150 mM NaCl) buffer with 2.5 mM CaCl₂ (final). When indicated, MPI 8 (20 μg/mL) or polyP (P700, 100 μM) were added. Control clots were prepared in the absence of MPI 8 or polyP. Clotting was initiated with 3 nM thrombin. Clots were then allowed to mature for 1 hour and processed for SEM imaging.

The clot samples were rinsed three times using HEPES buffer then fixed with Karnovsky fixative (2.5% glutaraldehyde and 4% formaldehyde). To allow for better penetration of the buffer solutions into the clot, a PELCO344I Laboratory Microwave System was used between buffer changes. After clot fixation, the clot sample was washed three times using fresh 0.1 M sodium cacodylate buffer before staining using 1% osmium tetroxide dissolved in 0.1 M sodium cacodylate buffer in the microwave. The clot sample was washed gently using distilled water for a minimum of five exchanges and resuspended in 50% ethanol solution. It was left to incubate for 10 minutes at room temperature before subjecting it to the microwave. The dehydration procedure was repeated using a graded series of ethanol solutions (70%, 80%, 90%, and 95% ethanol in water followed by 100% ethanol three times). Once the sample was fully dehydrated, it was placed in a Tousimis Autosamdri 815B Critical Point Dryer overnight under stasis mode before fully completing the drying process the following day. The processed samples were mounted on SEM stubs using hot glue and coated in 10 nm of Au/Pd coating (16.38 g/cm³) using a Cressington 208HR High Resolution Sputter Coater. Samples were stored in a desiccator. Clot images were captured on a Helios NanoLab 650 SEM at different magnifications (5000×, 10000× and 25000×). Multiple images from different areas of each clot were captured. Fiber diameters of clots were measured with ImageJ. For fibrin fiber diameter calculations, images from two independent experiments were analysed. Fibrin fiber diameters (n=80) from 4 separate areas of each clot were used to calculate the mean fiber diameter.

### Hemostasis as Measured in Laser-Ablation Saphenous Vein Model

Adult wild type mice (10-12 weeks old) were intravenously injected with 200 mg/kg MPI 8 or equivalent volume of PBS (vehicle) via tail vein injection. The effect of MPI 8 in hemostatic clot formation in vivo was examined using a laser-ablation saphenous vein hemostasis model as described. (Adili R. et al. A batroxobin containing activated Factor X effectively enhances hemostatic clot formation and reducing bleeding in hypocoagulant conditions in mice. Clin Appl Thromb Hemost. 2021 January-December; 27:10760296211018510, and Adili R. et al. First selective 12-LOX inhibitor, ML355, impairs thrombus formation and vessel occlusion in vivo with minimal effects on hemostasis. Arterioscler Thromb Vasc Biol. 2017 October; 37(10):1828-1839.) Mice were anesthetized by an intraperitoneal injection of ketamine/xylazine (100 and 10 mg/kg, respectively) and intravenously administered DyLight 488-conjugated rat anti-mouse platelet GP1bβ antibody (0.1 μg/g; EMFRET Analytics) and Alexa Fluor 647-conjugated anti-fibrin (0.3 g/g) via tail vein cannula. The saphenous vein was surgically prepared under a dissecting microscope and superfused with preheated bicarbonate saline buffer throughout the experiment. Blood flow of the saphenous vein was visualized under a 20× water immersion objective using a Zeiss Axio Examiner Z1 fluorescent microscope equipped with a solid laser launch system. The saphenous vascular wall was exposed to 2 maximum-strength 532-nm laser pulses (70 IJ; 100 Hz; for about 7 ns, 10 ms intervals) to puncture a hole (48 to 65 μm in diameter) in the vessel wall, resulting in bleeding visualized by the escape of fluorescent platelets to the extravascular space. The laser injury was performed at 30 seconds and repeated 5 and 10-minutes after the initial injury at the same site to assess platelet-fibrin hemostatic clot formation. The dynamics of platelet accumulation and fibrin deposition within the clot were recorded in real-time and the changes in the mean fluorescent intensity over time were analyzed using the Slidebook 6.0 program. A total of 4 mice (2 males and 2 females) with 3-4 independent injuries each were analyzed in the control and treatment groups.

### MPI Effects in Mice

Influence of MPI on Bleeding in Mice without Added polyP

A mouse model was used to assess the effect of MPI on bleeding in the absence of added polyP or any anticoagulants. Heparin was used as a positive control and saline was used as a negative control. Eight to ten week-old C57/BL6 mice were obtained from The Jackson Laboratories (Bar Harbor, ME), and the experimental protocol was approved by the International Animal Care and Use Committee at the University of Michigan. Mice were anesthetized, weighed and placed on a heated surgical tray. The tail was immersed into 15 mL of pre-warmed (37° C.) sterile saline (0.9% NaCl). To test the bleeding effects of different agents, MPIs, UHRA-10, saline or heparin were injected retro-orbitally and allowed to circulate for 5 minutes using solutions of MPI 1, MPI 6, MPI 8, UHRA-10 and UFH in sterile saline for maximum injection volumes of 50 μL and final concentrations of 100-300 mg/kg, or 200 U/kg for UFH. The distal tail (5 mm from the tip) was amputated with a surgical blade (Integra Miltex) and immediately re-immersed in 15 mL of pre-warmed (37° C.) sterile saline (0.9% NaCl). The time required for spontaneous bleeding to cease was recorded. After a maximum of 10 minutes, the tail was removed from the saline and the mouse was euthanatized by cervical dislocation. The blood samples were then pelleted at 500× g for 10 minutes at room temperature and the pellet was resuspended in 5 mL of Drabkin's Reagent (Sigma) and incubated at room temperature for 15 minutes. The amount of hemoglobin lost was quantified by comparing the absorbance of the samples at 540 nm to a standard curve of bovine hemoglobin in Drabkin's reagent.

### Inhibition of Thrombosis by MPI Via Intravital Microscopy Analysis by Laser Injury Thrombosis

A laser injury thrombosis model in mice was used to screen the efficiency of the MPIs. Intravital microscopy was used to measure the accumulation of platelets and fibrin at the site of the injury. Ten to twelve week-old C57/BL6 mice were obtained from The Jackson Laboratories (Bar Harbor, ME). The experimental protocol was approved by the International Animal Care and Use Committee at the University of Michigan. Male adult mice were anesthetized and a tracheal tube was inserted to facilitate breathing. Antibodies, anesthetic reagent (pentobarbital, 0.05 mg/kg body wt; Abbott Laboratories, Toronto, Ontario, Canada), and exenatide (60 nmol/kg body wt i.v.) were administered by a jugular vein cannula. The cremaster muscle was prepared under a dissecting microscope and superfused throughout the experiment with preheated bicarbonate buffer saline. Platelets were labeled by injecting a DyLight 649-conjugated rat anti-mouse GP1bb antibody (0.1 mg/g; EMFRET Analytics). Multiple independent upstream injuries were performed on a cremaster arteriole with the use of an Olympus BX51WI Microscope with a pulsed nitrogen dye laser. The dynamic accumulation of fluorescently labeled platelets within the growing thrombus was captured and analyzed using SlideBook software (Intelligent Imaging Innovations). Blood glucose levels were monitored throughout the experiment and remained constant.

### Inhibition of Thrombosis by MPI Via FeCl₃ Induced Injury to Carotid Arteries

Mice were anesthetized by an inhaled isoflurane-oxygen mixture. MPI and UHRA compounds diluted in sterile normal saline were injected retro-orbitally. The left carotid artery was exposed via a midline cervical incision and blunt dissection, and blood flow was monitored with a Doppler vascular flow probe (Transonic 0.5PSB) connected to a perivascular flowmeter (Transonic TS420). To induce thrombosis, two 1×2-mm pieces of filter paper (Whatman GB003) saturated with freshly prepared 7.5% anhydrous FeCl₃ in 0.9% saline were applied to the deep and superficial surfaces of the artery. After 3 minutes, the filter papers were removed, and the vessel was irrigated with saline. Blood flow was monitored from FeCl₃ application for 30 minutes or until occlusion, defined as no detectable flow for 3 minutes. Mice were then euthanized by cervical dislocation while still under anesthesia. Flow data were interpreted with LabScribe2 (iWorx Systems). Data for this study are reported as mean (n=8 mice)±standard deviation.

### Tolerance of MPI 8

### Acute Toxicity in Mice

An escalating dose study in mice was used to measure the tolerance of MPI 8. Female Balb/C mice (6-8 weeks, 20-26 g) were individually weighed and were divided into groups of 4 for each dose. Each group of mice (N=4) were administered study compounds intravenously (via tail vein) with increasing doses of MPI 8 (250 to 500 mg/kg). The injection volume was 200 μL/20 g mouse. The mice were briefly restrained (less than 30 sec) during i.v. injections. Dilation of the vein was achieved by holding the animals under a heat lamp for about 1-2 min. After injection, the mice were returned to the cages and monitored for signs of acute toxicity over a period of 1 day. Body weights of individual mice were recorded prior to injection. After 24 hours of injection, mice were terminated by C02 asphyxiation, blood (50 μL) was collected from each mouse on the final day and necropsy was performed on all animals. Serum samples were analyzed for lactate dehydrogenase (LDH), aspartate aminotransferase (AST) and alanine aminotransferase (ALT) activity.

### Analysis of LDH Activity in Mouse Serum Samples

Serum samples were analyzed for LDH activity using a lactate dehydrogenase enzyme assay kit (Abcam., Cambridge, UK). The kit measures the concentration of LDH using a direct, plate-based, colorimetric titration and consists of a 96-well microtiter plate, LDH reagent mix, standard and standard dilution buffer. When serum is added to the LDH reagent mix, the LDH in the sample converts the lactate and NAD⁺ in the mix to pyruvate and NADH, which interacts with a specific probe to produce a color which can be monitored by measuring the increase in the absorbance of the reaction at 450 nm over a 5 min time interval. In a typical test procedure, 5 μL of the serum sample (dilution factor determined upon initial reading) was added in duplicate to microplate wells and incubated with 50 μL of the reconstituted LDH per the supplier's instructions, and the absorbance was measured at 450 nm. A calibration curve was created using standards of NADH from 0-12.5 nmol/well. The average value of the absorbance was used in combination with the standard curve to obtain the LDH activity (IU/mL).

### Analysis of AST Activity in Mouse Serum Samples

Serum samples were analyzed for AST activity using an aspartate aminotransferase enzyme assay kit (Sigma Aldrich., Oakville, ON). The kit measures the concentration of AST using a direct, plate-based, colorimetric titration and consists of a 96-well microtiter plate, AST reagent mix, standard and standard dilution buffer. When serum is added to the AST reagent mix, the AST in the sample transfers an amino group from aspartate to α-ketoglutarate resulting in oxaloacetate and glutamate, which results in the production of a colorimetric product proportional to the AST enzymatic activity present. This activity is monitored by measuring the increase in the absorbance of the reaction at 450 nm over a 30 min time interval. In a test procedure, 50 μL of the serum sample (dilution factor determined upon initial reading) was added in duplicate to microplate wells and incubated with 100 μL of the reconstituted AST reaction mixture per the supplier's instructions, and the absorbance was measured at 450 nm. A calibration curve was created using standards of glutamate from 0-10 nmol/well. The average value of the absorbance was used in combination with the standard curve to obtain the AST activity (IU/mL).

### Analysis of ALT Activity in Mouse Serum Samples

Serum samples were analyzed for ALT activity using an alanine aminotransferase enzyme assay kit (Sigma Aldrich., Oakville, ON). The kit measures the concentration of ALT using a direct, plate-based, colorimetric titration and consists of a 96-well microtiter plate, ALT reagent mix, standard and standard dilution buffer. When serum is added to the ALT reagent mix, the ALT in the sample transfers an amino group from alanine to α-ketoglutarate resulting in pyruvate and glutamate, which results in the production of a colorimetric product proportional to the ALT enzymatic activity present. This activity is monitored by measuring the increase in the absorbance of the reaction at 570 nm over a 30 min time interval. In a test procedure, 20 μL of the serum sample (dilution factor determined upon initial reading) was added in duplicate to microplate wells and incubated with 100 μL of the reconstituted ALT reaction mixture per the supplier's instructions, and the absorbance was measured at 570 nm. A calibration curve was created using standards of pyruvate from 0-10 nmol/well. The average value of the absorbance was used in combination with the standard curve to obtain the ALT activity (IU/mL).

### Chronic Toxicity of MPI 8

An escalating dose study in mice was used. Female Balb/C mice (6-8 weeks, 20-26 g) were individually weighed and were divided into groups of 4 for each dose. Each group of mice (N=4) were administered test compounds intravenously (via tail vein) with increasing doses of MPI 8 (100 to 500 mg/kg). The injection volume was 200 μL/20 g mouse. The mice were briefly restrained (less than 30 sec) during i.v. injections. Dilation of the vein was achieved by holding the animals under a heat lamp for about 1-2 min. After injection, the mice were returned to cages and monitored daily for signs of acute toxicity over a period of 14 days. Body weights of individual mice were recorded prior to injection and every day except weekends thereafter. On day 15, mice were terminated by C02 asphyxiation, blood (50 μL) was collected from each mouse on the final day and necropsy was performed on all animals. Serum samples were analyzed for lactate dehydrogenase (LDH) activity using a lactate dehydrogenase enzyme assay kit (Abcam., Cambridge, UK) as described in above.

In Vivo Studies with MPI 3

### Cecal Ligation and Puncture (CLP) Procedure

The CLP model was adapted from established protocols in the literature. (Toscano, M. et al. (2011). Cecal ligation puncture procedure. JoVE (Journal of Visualized Experiments), (51), e2860.) Nine-week-old female mice (C57BL/6NCrl) were obtained from Charles River Laboratories. To gain access to the peritoneal cavity, a small incision was made in the abdomen. The cecum was located and exteriorized with wetted cotton tips. On the tip of the cecum, a ligature close to the size of a pinhead was made. A 19G needle was used to puncture the ligated cecum, and some feces were externalized. After that, the cecum was returned to the abdominal cavity, and the muscle and skin layers were closed with a Vicryl 6×0 running suture. For SHAM controls, the same procedure was followed except for the ligature and puncture in the cecum. Buprenorphine 0.1 mg/kg was administered 4 hours after surgery. MPI 3 at concentrations of 25 or 50 mg/kg were given subcutaneously at 2 hours, 4 hours, and 6 hours (3 doses) after surgery. Mice were euthanized 8 hours after surgery, and blood was collected via cardiac puncture into a syringe containing 3.2% sodium citrate.

### Mouse Plasma Analysis

Citrated blood was centrifuged at 1200×g for 10 minutes to remove RBCs, then at 10,000×g for 10 minutes to remove any residual cells and debris, thereby yielding platelet-poor plasma (PPP). Calibrated automated thrombography (CAT) was used to measure thrombin generation. In HEPES buffer, 80 μL of 1:4 diluted mouse plasma was mixed with 20 μL of the PPP—reagent LOW (Stago). The addition of 20 μL of FluCa reagent containing 2.5 mM Fluorogenic Urokinase Substrate III (Calbiochem, CAT #672159) and 100 mM CaCl₂) in HEPES-BSA buffer triggered thrombin generation. The CAT software was used to process and analyze the data. TAT complex was measured using the Mouse Thrombin-Antithrombin (TAT) Complex ELISA Kit (Abcam, CAT #ab137994) as directed by the manufacturer. DNA concentration in the plasma was determined using the Quant-iT™ PicoGreen™ dsDNA Assay Kit (Fisher Scientific, CAT #-P11496) as directed by the manufacturer.

### Mouse Plasma Analysis for Cytokine/Chemokine Profile

Blood was collected using the above-mentioned procedure and centrifuged at 1000×g for 10 minutes at 4° C. within 30 minutes of collection. The plasma was collected, diluted twice with phosphate-buffered saline (PBS), and stored at −80° C. Samples were sent to Eve Technologies to for a cytokine profile using their Mouse Cytokine/Chemokine 31-Plex Discovery Assay® Array (MD31).

### Statistical Analysis

Data are expressed as the mean±standard deviation from n (≥3) independent experiments unless otherwise specified. Statistical analyses were performed using GraphPad Prism version 7.0 software, using a Student's t-test or by one-way ANOVA followed by a Dunnett post hoc test. P values<0.05 were considered statistically significant. Samples were denoted as statistically significant. p<0.05 (*), p<0.01 (**), p<0.001 (***) and p<0.0001 (****). Experiments were performed in duplicate at least 3 times and results were pooled into a single dataset unless stated otherwise.

### Experimental Methods for Heparin Neutralization

### HPG-PEG Synthesis for MPI 2

A 3-neck round bottom flask was cooled under vacuum and filled with argon. To this, 1,1,1-tris(hydroxymethyl)propane (TMP, 167 mg) and potassium methylate (25 wt % solution in methanol, 0.110 mL) were added and stirred for 30 minutes. Methanol was removed under high vacuum for 4 hours. The flask was heated to 95° C. and distilled glycidol (3.8 mL) was added over a period of 15 hours. After complete addition of glycidol, the reaction mixture was stirred for an additional 12 hours. Then, mPEG₃₅₀-epoxide (10.5 mL) was added over a period of 12 hours at 95° C. The reaction mixture was stirred for additional 4 hours. The reaction was cooled to room temperature, quenched with methanol, then passed through Amberlite IR-120H resin to remove the potassium ions and twice precipitated from diethyl ether. The polymer was then dissolved in water and dialyzed in water membrane for 3 days with periodic changes in water.

¹H NMR (300 MHz, Chloroform-d) δ 4.04-3.40 (m, 50H), 3.38 (s, 3H). mPEG₃₅₀ content (by 1H NMR): 25 mol %; Polyglycerol: 75 mol %.

GPC-MALLS (0.1 M NaNO₃): Mₙ 24 000; Mw/Mₙ 1.3.

### HPG-mPEG-OTs Synthesis for MPI 2

This reaction was carried out under an inert gas atmosphere and exclusion of water. HPG-mPEG (200 mg) in a 3-necked 100 mL flask with thermometer and magnetic stirrer was dissolved in pyridine (5 mL). The solution was cooled to 0° C. by means of ice/NaCl bath, then, a solution of TsCl (1.2 eq. of target average number of groups) in pyridine (5 mL) was added dropwise so that the temperature did not exceed 5° C. The brown mixture was stirred for 16 hours as the reaction was allowed to warm to room temperature. Then solvent was removed in vacuo. The resulting mixture was then dialysed in water for 3 days with periodic changes in water to give a honey-like product.

¹H NMR (400 MHz, Chloroform-d): δ 7.78 (m, 0.7H), 7.34 (m, 0.7H), 3.98-3.39 (m, 48H), 3.35 (s, 3H), 2.45 (s, 1.05H).

### HPG-mPEG-NH₂ Synthesis for MPI 2

In a 50 mL q-necked flask with reflux condenser and magnetic stirrer was dissolved HPG-mPEG-OTs (200 mg, 0.20 mmol OTs-groups) in anhydrous 1,4-dioxane (10 mL). (Roller, ibid.) After addition of CBG (1 mmol, 5.0 eq.), the resulting suspension was heated to reflux at 115° C. for 24 hours. After cooling the reaction mixture, the mixture was dialyzed in water for three days with periodic changes in water to give a brown honey-like product.

¹H NMR (300 MHz, Chloroform-d): δ 3.89-3.43 (m, 35H), 3.38 (s, 3H), 2.87 (m, 9H).

### HPG-mPEG-NMe₂ Synthesis for MPI 2

Amine functionalized HPG-mPEG (200 mg, 24 CBG per polymer) was transferred to a 20 mL side-necked flask. The reaction mixture was dissolved in DI water at room temperature. 10 mL of a formic acid/formaldehyde (1:1) mixture was added dropwise to the reaction flask. The reaction was stirred and heated to reflux at 110° C. for 24 hours. After cooling the reaction mixture, the mixture was dialyzed in water for two days with periodic changes in water until the pH of the water was neutral. Upon lyophilizing to remove excess water, MPI 2 was collected.

¹H NMR (300 MHz, Chloroform-d): δ 8.11 (s), 4.11-3.19 (m), 3.19-2.95 (m), 2.78-1.94 (m).

### In Vitro Studies

Determination of Heparin Reversal Activity by Activated Partial Thromboplastin Time Assay (aPTT)

Stock MPI, UHRA or protamine sulfate solutions were prepared in 20 mM HEPES buffer (pH 7.4 and 150 mM NaCl). Unfractionated heparin (Fresenius Kabi, Canada) or tinzaparin (LEO Pharma, Canada), 4 U/mL and 1 U/mL final concentrations, respectively, were incubated with sodium citrate anticoagulated pooled normal plasma (Affinity Biologicals, Canada) to prepare heparinized plasma, and were used to study the neutralization activity by various MPI and UHRA constructs. The neutralization activity of MPI, UHRA and protamine sulfate on the coagulation cascade was examined by mixing 20 μL of MPI or UHRA or protamine solution with 180 μL of heparin derivative incubated plasma ( 1/10 v/v). The final concentration of MPI, UHRA and protamine in plasma ranged from 0.025 mg/mL to 0.25 mg/mL. 200 μL of aPTT reagent (Dade®Actin®FS Activated PTT, Siemens/Dade-Behring) was then added to the neutralization reagent-plasma sample and 100 μL of this resulting mixture was transferred to cuvette-strips and incubated at 37° C. for 3 minutes. The clotting time was measured on a STart® 4 coagulometer (Diagnostica Stago, France) and began when 50 μL of 25 mM CaCl₂) was added into each cuvette. Saline added to plasma with and without heparin-derivatives was used as a control for the experiments. The percentage of neutralization was calculated from the difference in the clotting times observed for MPI/UHRA/protamine versus control saline and heparinized plasma. All experiments were performed with pooled plasma of 20 donors in triplicates and the average values (mean±SD) are reported.

### Effect of Heparin Reversal on Thrombin Generation by Calibrated Automated Thrombography

A thrombin generation assay was carried out at 37° C. by measuring the fluorescence intensity upon cleavage of a fluorogenic substrate, Z-Gly-Gly-Arg-AMC by the regenerated thrombin. Commercially available pooled platelet normal plasma (PNP, 30 donors) from George King Bio-Medical, USA was mixed 1:1 with HBS (20 mM HEPES with 100 mM NaCl at pH 7.4). Phosphatidylcholine (80): phosphatidylserine (20) (PCPS) liposomes were added to obtain a final concentration of 25 μM. Serial dilutions of MPI in HBS were prepared fresh each for each experimental replicate and two technical replicates were performed each time. Plasma-liposomes incubated with thrombin-α2-macroglobulin were used as a thrombin calibrator. The thrombin generation assay was initiated by addition of fluorogenic substrate in 60% BSA in HEPES buffer and CaCl₂) (0.1M final concentration) to each well with a multichannel pipette. Substrate hydrolysis was monitored with Thrombinoscope™ plate reader. Fluorescence intensity was recorded at 37° C. every 30 seconds over a period of 1.5 hours and analyzed using Thrombinoscope™ software from Diagnostica Stago. For determination of heparin neutralization efficacy, the specific heparin tested would be added directly to the warmed PNP at 37° C. Only the type of heparin was varied: UFH, enoxaparin and Fondaparinux. TF (Thrombinoscope™) was added to initiate clotting at a final concentration of 5 μM.

### Platelet Activation Analysis by Flow Cytometry

The level of platelet activation was quantified by flow cytometry. Ninety microliters of PRP were incubated at 37° C. with 10 μL of stock MPI samples for one hour. Ten microliters of post-incubation platelet/polymer mixture were diluted in 45 μL PPP and incubated for 15 minutes in the dark with 5 μL of monoclonal anti-CD62-PE (Immunotech, Marseille, France). The reaction was then stopped with 0.5 mL of phosphate-buffered saline solution. The level of platelet activation was analyzed in a BD FACS Canto II flow cytometer (Becton Dickinson, ON, Canada) by gating platelet-specific events based on their light scattering profile. Activation of platelets was expressed as the percentage of platelet activation marker CD62P-PE (phycoerythrin) fluorescence detected in the 10,000 total events counted. Measurements were performed with PRP from three different donors and mean of which was recorded. Thrombin receptor activator receptor 6 (TRAP6), a known platelet activator (Sigma Aldrich, Oakville, ON, Canada) was used as a positive control for the flow cytometric analysis.

### Whole Blood Coagulation Analysis by Thromboelastometry

Whole blood collected in sodium citrate anticoagulant mixed with MPI in HEPES buffered saline (HBS) was subjected to a coagulation study using ROTEM® delta from Tem Innovations GmbH (Instrumentation Laboratory as of 2016) at 37° C. Stock solutions of MPI and UHRA were prepared at concentrations 100× the final desired concentration in HBS. Citrate anticoagulated whole blood (356 μL) collected from normal volunteers after informed consent and using an approved UBC protocol was mixed with 44 μL of MPI, UHRA or HBS mixture. The blood mixture (340 μL) was transferred into the ROTEM cup and was re-calcified with 20 μL of 0.2 M calcium chloride solution. As a negative control, HBS (20 mM HEPES+150 mM NaCl) mixed with whole blood was used as control for the experiment. The experiments were repeated with at least 5 different donors and average values are reported. For heparin reversal studies, UFH was included in the 44 μL mixture for a final concentration of 0.5 U/mL and as a positive control no MPI was added. To prepare heparinized blood, 10 000 U/mL stock UFH was added to 4 mL whole blood at 37° C. for a final concentration of 0.5 U/mL. All the experiments were initiated within 10 minutes of blood collection and the clot characteristics such as clotting time and clot strength were measured from the ROTEM generated. Whole blood collected from five donors was used and the mean value with standard deviation was reported.

In Vivo Studies with MPI 2

### Mouse Tail Bleeding Model

C57/BL6 mice (8- to 10-week-old) were purchased from The Jackson Laboratories (Bar Harbor, ME), and the experimental protocol was approved by the International Animal Care and Use Committee at the University of Michigan. Mice (N=8) were anesthetized, weighed, and placed on a heated surgical tray. The tail was immersed into 15 mL of pre-warmed (37° C.) sterile saline (0.9% NaCl). To test the bleeding side effects, MPI 2, saline, and heparin, were injected retro-orbitally and allowed to circulate for 5 minutes. The distal tail (5 mm from the tip) was amputated with a surgical blade (Integra Miltex) and immediately re-immersed in 15 mL of pre-warmed (37° C.) sterile saline (0.9% NaCl). The time required for spontaneous bleeding to cease was recorded. After a maximum of 10 minutes, the tail was removed from the saline and the mouse was euthanatized by cervical dislocation. The blood samples were then pelleted at 500 g for 10 minutes at room temperature, the pellet was resuspended in 5 mL of Drabkin's Reagent (Sigma), and then incubated at room temperature for 15 minutes. The amount of hemoglobin lost was quantified by comparing the absorbance of the samples at 540 nm to a standard curve of bovine hemoglobin in Drabkin's reagent.

### Mouse Tail Bleeding Model for Heparin Reversal Activity by MPI 2

To test the ability of MPI 2 to reverse the activity of heparins in mice, the same mouse tail bleeding model as described above was used, except for the following exception. Mice were first injected retro-orbitally with either unfractionated heparin (200 U/kg) or enoxaparin (200 U/kg) which was allowed to circulate for 5 minutes. Mice were then injected retro-orbitally with either MPI 2, or saline, circulated for 5 minutes prior to tail tip amputation. All other experimental details were the same.

### Nucleic Acid Inhibition

### Inhibition of Plasma Clotting

Inhibitors were incubated with 50% plasma spiked with 67.11 μg/mL of LMW poly IC for 45 minutes at room temperature followed by 3 minutes incubation at the 37° C. Clotting was triggered in Stago Start 4 coagulometer by 10 mM calcium, 1:15000 diluted re-lapidated tissue factor and PCPS vesicles. For concentration dependent studies, 20 μg/mL HMW poly IC was used.

### Reversal of FXII Activation by Nucleic Acids

Inhibitors were incubated with 50% plasma spiked with 100 g/mL of HMW poly IC for 45 minutes. S2302 substrate activity in plasma was monitored by measuring absorbance for 1 hr at 37° C. at 405 nm. Initial maximum velocity of the reaction was calculated and plotted with concentration.

### Reversal of Nucleic Acid Mediated Thrombin Generation by Calibrated Automated Thrombography

Thrombin generation was measured by calibrated automated thrombography. Commercially available pooled platelet normal plasma (PNP, 30 donors) from George King Bio-Medical, USA was incubated with nucleic acids with or without the inhibitor at 37° C. for 45 minutes. 80 μL of this mixture was mixed with 20 μL of Stago's PPP low reagent in clear round-bottom immuno 96-well plates from Thermo Fisher. Thrombin generation was triggered using 20 μL of FluCa reagent and measured in a thrombinoscope.

### Reversal of Nucleic Acid Mediated Anti-Fibrinolysis

Plasma clot lysis assays were performed by triggering 30% commercially available pooled platelet normal plasma (PNP, 30 donors) from George King Bio-Medical, USA with 0.04IU/mL thrombin, 20 mM calcium and 70 ng/mL tissue Plasminogen Activator(tPA). The lysis was monitored by measuring absorbance at 405 nm for 10 hrs.

### Human Subjects

The protocol for blood collection from human subjects in the Centre for Blood Research at the University of British Columbia has been approved by the Institutional Review Board (IRB) within the University of British Columbia (UBC Ethics approval no: H10-01896) with written consent obtained from donors.

### Blood Collection and Plasma Preparation

Blood was drawn from consenting informed healthy volunteer donors at Centre for Blood Research, University of British Columbia, in BD Vacutainer® Citrate Tubes containing 3.2% buffered sodium citrate solution. Blood was centrifuged at 150×g for 10 minutes to separate platelet-rich plasma (PRP), and then spun at 1000×g for 15 minutes for platelet-poor plasma (PPP). Pooled normal plasma (PNP) from 20 donors was purchased from Affinity Biologicals (ON, Canada).

Specificity of MPI 8 Toward polyP in ADP-Mediated Platelet Activation

Human whole blood collected was into 3.8% sodium citrate tubes. Tubes were then centrifuged at 156×g for 12 minutes to generate platelet-rich plasma (PRP). The influence of MPI 8 on ADP-mediated platelet activation was assessed in 2 ways. In both cases, the final concentration of ADP was 40 μM. In the first approach, MPI 8 was pre-mixed with ADP (10 μL total) immediately before the addition of 90 μL of PRP, and the resulting suspension was incubated for 15 minutes at 37° C. In the second, PRP was pre-incubated with MPI 8 for 1 hour at 37° C. before activation with ADP (15 minutes at 37° C.). Controls containing PBS alone (vehicle) as well as ADP without MPI 8 were also included. Activation was then assessed by flow cytometry (CytoFLEX Flow Cytometer, Beckman Coulter). Briefly, 5 μL of PRP suspension was added to 50 μL of PBS containing 20×-diluted anti-human CD62P-PE (BD Biosciences). Platelets were gated based on anti-human CD42-FITC (BD Biosciences), prepared in the same manner. Using this gate, 10,000 events were counted, and activation was quantified by the percentage of cells positive for CD62P.

### Example 1

### Cationic Binding Group (CPG) Structures

The structure of the macromolecular polyanion inhibitors (MPIs) of the present invention provides 2 components: A charge switchable cationic binding group (CBG); and a biocompatible scaffold as illustrated in FIG. 1. To select CBGs to efficiently bind polyP, two competing factors are considered: 1) the need for a sufficiently high cationic charge density of the ligand at physiological pH (7.4) to initiate binding to polyanionic polyP; and 2) the need to keep the total quantity of charge on the MPI molecule low at physiological pH (7.4) to increase biocompatibility by reducing non-specific binding. To achieve these properties, the switchable protonation behavior of weakly basic amines was exploited in the development of CBGs of the present invention.

Two novel cationic binding groups were identified based on a comparison of multiple synthetic and biologically derived amine structures reported in the literature (Table 1): CBG I (linear amine with two-carbon alkyl linker) and CBG II (linear amine with three-carbon alkyl linker) are depicted in FIG. 1. These new binding groups were chosen because they fit the initial selection criteria: each binding group has at least one amine residue with pKₐ >8, thereby ensuring at least one cation per binding group at physiological pH and that some cations are present under physiological pH to initiate the polyP binding. The remaining amines on these binding groups preferably also have pKₐ of approximately 6-7, resulting in partial double protonation of the CBGs. The resulting CBGs have protonation states that can be switched due to their high dependence on the local environment of the CBGs allowing them to exhibit higher charge density when local dielectric conditions are modified, for example, the binding of a polyanion such as polyP. While many structures may give appropriate charge density at physiological pH, alkyl amines similar to N,NN′,N″,N″-pentamethyldiethylenetriamine (PMDTA) provide cationic amine residues with pKₐ values appropriate for use as a switchable CBG. (Smith, R. M. and Martell, A. E. Critical Stability Constants; Springer US: Boston, MA, 1975. https://doi.org/10.1007/978-1-4613-4452-0.) The pKₐ of adjacent amine residues on the same CBG is highly dependent on the chain length of spacer between the charges, resulting in a wide range of possible protonation behavior upon binding. As such, an analogue with an additional carbon spacer between the amines was also chosen. The protonation state is highly dependent on adjacent cationic residues due to the strong electrostatic repulsion between positive charges spaced one to three carbons and thus can change upon binding to the desired polyanion. (Rodrigo, A. C. et al. Self-Assembling Ligands for Multivalent Nanoscale Heparin Binding. Angew. Chem. Int. Ed. 2011, 50 (20), 4675-4679., Udit, A. K. et al. Heparin Antagonism by Polyvalent Display of Cationic Motifs on Virus-Like Particles. ChemBioChem 2009, 10 (3), 503-510., and Cascio, S. et al. Protonation of Polyamines in NaCl Aqueous Solution and Binding of Cl— by Polyammonium Cations. Fluid Phase Equilibria 2000, 170 (2), 167-181.)

### Example 2

### Scaffolds for Presentation of CBGs

Preferred polymer scaffolds covalently conjugate to the selected CBGs in the MPIs. We created a library of MPIs with a varying number of different CBGs on different scaffold sizes, to identify the optimal protonation pattern for MPIs to bind polyP. We have identified a scaffold with hyperbranched polyglycerol (HPG) core attached with a swollen PEG corona that accounts for approximately 30 mol % of final copolymer scaffold (HPG-PEG). The PEG corona provided sufficient graft density to generate brush layer to prevent non-specific interactions. (Shenoi, ibid, Kalathottukaren and Abbina, ibid.) Selection of this HPG-PEG scaffold allows testing of the switchable protonation state and local recruitment of protons by CBGs upon its binding to polyP without the influence of other factors.

### Example 3

### MPI Library Synthesis and Characteristics

Two distinct HPG-PEG core scaffold sizes with 10 kDa and 20 kDa molecular weight were developed. Post functionalization of the HPG-PEG scaffold with different CBG groups at different densities provided the generation of a library of the MPI candidates for evaluation (Table 2).

TABLE 2
Library of MPIs detailing the structural characteristics.

| | Polymer | Avg. | Avg. number | |
|---|---|---|---|---|
| | backbond^{a} | number of R | of charges at | |
| | (kDa) | groups^{b} | pH 7.4^{c} | R group structure |
| MPI 1 MPI 2 MPI 3 | 21 23 21 | 18 24 31 | 23 35 42 | 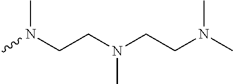 |
| MPI 4 MPI 5 | 23 21 | 24 30 | 26 32 | 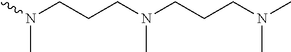 |
| MPI 6 MPI 7 | 10 10 | 11 15 | 16 20 | 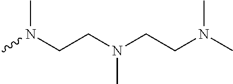 |
| MPI 8 MPI 9 | 10 10 | 11 15 | 12 16 | 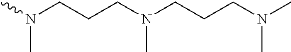 |
| UHRA | 21 | 28 | 45 | 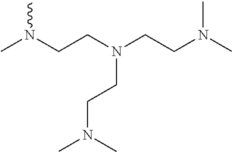 |

^{a}Obtained from GPC-MALLS.
^{b}Obtained from conductometric titrations.
^{c}Obtained from potentiometric titrations.

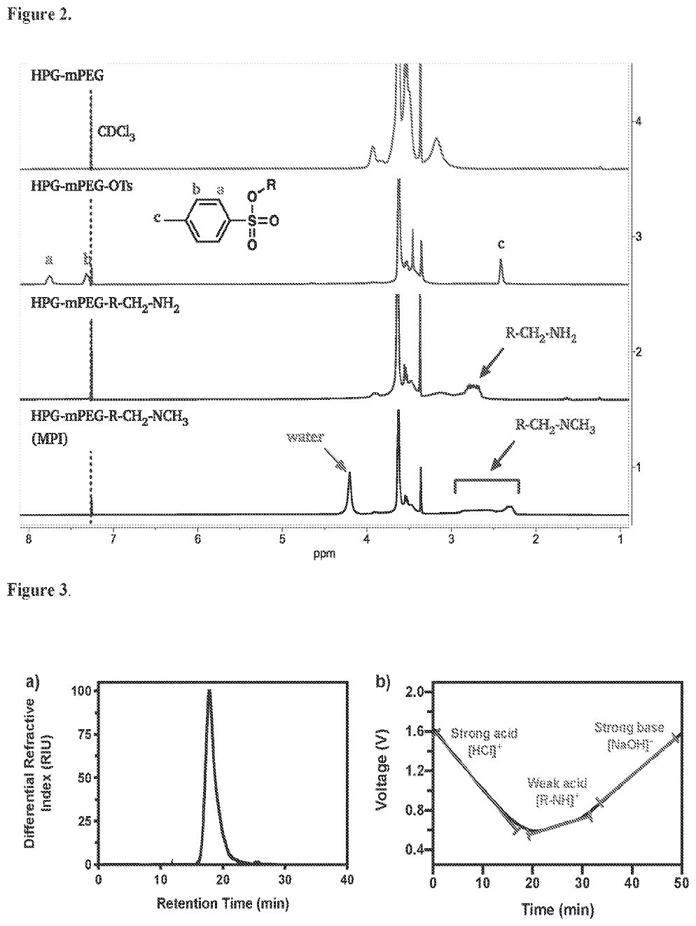

Another polycationic polymer was prepared from our generation of compounds (UHIRA), to serve as a reference from which the improved activity and increased biocompatibility of the novel MIPI candidates can be measured. Detailed characteristics of the MPI candidates including their NMR spectra, conductometric titration curve, GPC profiles are provided in FIGS. 2 and 3. The candidates were selected to fully bracket the effects of several variables: CBG structure, CBG doping concentration, and scaffold molecular weight. By choosing the chemistry and density of the CBGs on MIPIs, we generated an inhibitor with enhanced specificity towards polyP having low charged state at physiological pH. By varying multiple factors, we determined the effect of CBGs on binding activity and equilibrium protonation behavior while also determining how charge spacing and density affect biocompatibility of the MPIs and its binding properties to polyP. Using this library, the interactions between the optimal polycation-polyanion pair were studied, using an optimized number of CBGs to maximize the number of binding events between the MIPI and its polyanionic partner (polyP) while minimizing nonspecific interactions between the MIPIs and other anionic macromolecules present in the blood. (Laucirica, G. et al. Dangerous Liaisons: Anion-Induced Protonation in Phosphate-Polyamine Interactions and Their Implications for the Charge States of Biologically Relevant Surfaces. Phys. Chem. Chem. Phys. 2017, 19 (12), 8612-8620.)

### Example 4

### Protonation of MPIs

Using CBGs with multiple alkylamines with different protonation constants, MPIs were generated whose protonation state was switchable, presenting a low charge density at physiological pH but adopting a highly charged state when binding to polyP due to the highly anionic microenvironment surrounding the polyP partner after the binding event. These MPIs provide the solution with one or two amine residues on CBG protonated prior to polyP binding are able to support up to three charges per CBG with an energetic incentive to adopt this conformation. (Gohlke, H. and Klebe, G. Approaches to the Description and Prediction of the Binding Affinity of Small-Molecule Ligands to Macromolecular Receptors. Angew. Chem. Int. Ed. 2002, 41 (15), 2644-2676., and Barril, S et al. Salt Bridge Interactions: Stability of the Ionic and Neutral Complexes in the Gas Phase, in Solution, and in Proteins. Proteins 1998, 32 (1), 67-79.)

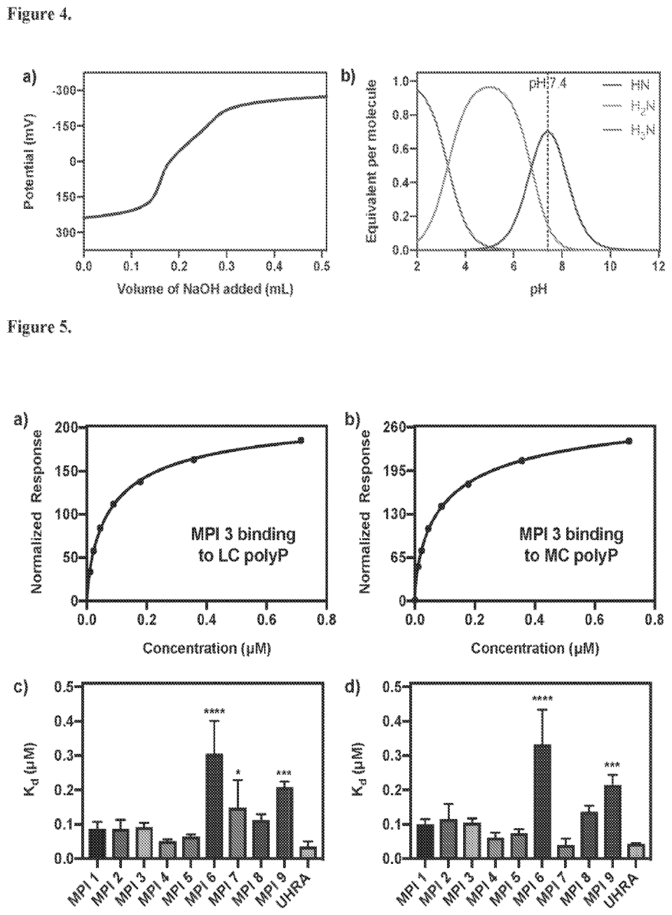

The pKₐ values of the amines on the overall MPI structure determined the ability of each amine on the final MPI structure to accept a proton, depending on its local electronic environment. Each CBG candidate was carefully selected for protonation properties. Both CBG I and CBG II present two amines with pKₐ >7.4, indicating the likelihood of being protonated at physiological conditions. The 2 amines are on the extremities of the CBG structure (FIG. 1). Cationic charges separated by an ethyl spacer display a strong repulsion, resulting in a depression of the pKₐ value. (Cascio, ibid.) Thus, the central amine possesses the lowest pKₐ value. As seen from the addition of one extra carbon between the amines (propyl instead of ethyl) in CBG II structure, the central amine pKₐ increases by 2. As such, a change in each ligand's microenvironment upon attachment to the HPG-PEG scaffold is expected. Using potentiometric studies, the overall average protonation strength of the CBGs within MPI was obtained. The number of CBGs per MPI, obtained from conductometric titration, as well as the average charge state or number of cations present on each MPI at physiological pH, calculated using the Henderson-Hasselbalch equation (Eq. 1) from potentiometric titrations (FIG. 4 and Table 3) is given in Table 2. These titrations demonstrate the tunable number of CBGs across MIPIs generated, and also highlight the reduced charge present at physiological pH for CBG I and CBG II compared to methylated TREN.

$$p H = p {K}_{a} + {log}_{10} \frac{[ {A}^{-} ]}{[ H A ]} \qquad Eq . ( 1 )$$

TABLE 3
Summary of log K values obtained from potentiometry
for various CBGs and when loaded on various MPIs.

| | L + | HL + | H₂L + | H₃L + |
|---|---|---|---|---|
| | H [symbol] HL | H [symbol] H₂L | H [symbol] H₃L | H [symbol] H₄L |
| CBG I⁵¹ | 9.22 ± 0.02 | 8.41 ± 0.02 | 2.09 ± 0.02 | — |
| MPI 1 | 8.1 ± 0.1 | 7.0 ± 0.3 | 3.7 ± 0.1 | — |
| MPI 2 | 8.7 ± 0.4 | 7.5 ± 0.2 | 4.3 ± 0.1 | — |
| MPI 3 | 8.7 ± 0.4 | 7.1 ± 0.2 | 3.4 ± 0.5 | — |
| CBG 2 | 10.6 ± 0.2 | 8.3 ± 0.2 | 4.1 ± 0.1 | — |
| MPI 4 | 8.9 ± 0.2 | 6.5 ± 0.3 | 3.6 ± 0.1 | — |
| MPI 5 | 9.0 ± 0.1 | 6.5 ± 0.2 | 3.1 ± 0.3 | — |
| Me₆- | 9.50 | 8.68 | 7.44 | <1.0 |
| TREN⁷⁹ | | | | |
| UHRA | 9.2 ± 0.2 | 7.5 ± 0.1 | 4.1 ± 0.1 | <1.0 |

Potential in mV was measured as standardized base (0.15 M NaOH) was titrated into acidified (pH 2) solution of CBG or MI at 25° C., 160 mM NaCl.

### Example 5

### PolyP Binding Characteristics of the MPI Library Via Surface Plasmon Resonance (SPR) Analysis

After determining the structure and characterization of protonation of the MIPIs, we investigated their polyP inhibition capabilities. Using surface plasmon resonance (SPR), the binding affinity of each MPI towards two chain lengths of surface bound polyP long chain (LC) and medium chain (MC) polyP, long chain (LC) polyP (characterized as having hundreds to thousands of phosphate units, in this case a mode of 1070 phosphate units), and MC polyP approximately half the size with a mode of 560 phosphate units, were compared in this high-throughput experiment. Results are provided in Table 4 and FIG. 5.

TABLE 4
Summary of binding affinity obtained for each MPI
candidate binding to 3 surface bound polyP chain
lengths (P1070, P560, P110). SPR was run at 25°
C. in 20 mM HEPES running buffer with 140 mM NaCl.

| MPI | K_{d} (nM) | | |
|---|---|---|---|
| Label | P1070 | P560 | P110 |
| MPI 1 | 86 ± 21 | 100 ± 16 | 141 ± 12 |
| MPI 2 | 86 ± 26 | 114 ± 44 | 186 ± 90 |
| MPI 3 | 91 ± 12 | 105 ± 12 | 75 ± 36 |
| MPI 4 | 50 ± 6 | 62 ± 14 | 92 ± 22 |
| MPI 5 | 63 ± 6 | 75 ± 10 | 82 ± 19 |
| UHRA-8 | 35 ± 15 | 43 ± 2 | 3 ± 4 |
| MPI 6 | 305 ± 96 | 332 ± 100 | 388 ± 108 |
| MPI 7 | 149 ± 80 | 40 ± 19 | 198 ± 63 |
| MPI 8 | 112 ± 17 | 136 ± 17 | 37 ± 33 |
| MPI 9 | 208 ± 17 | 215 ± 30 | 193 ± 134 |
| UHRA-10 | 161 ± 7 | 146 ± 18 | 95 ± 52 |

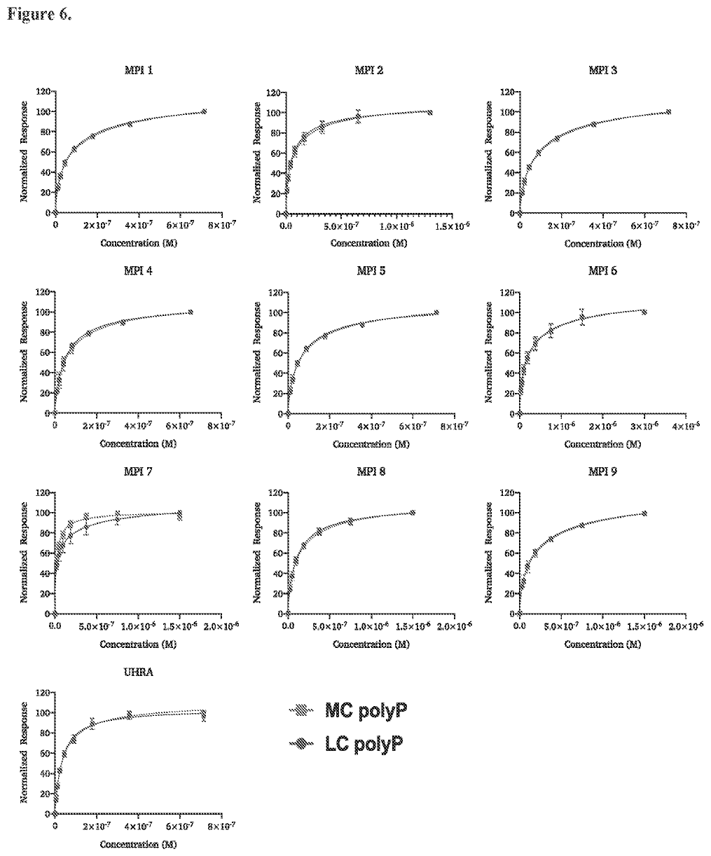

FIGS. 5a and 5b show the SPR binding curve of an MPI candidate (MPI 3) with LC and MC polyP respectively. Representative binding profiles are provided in FIG. 6. Data summarizing average binding affinity for all MPI candidates in comparison with UHRA is given in FIGS. 5c and 5d. The measured MPI compounds exhibited dissociation constant (K_{d}) values in the sub-micromolar range against surface bound polyPs in HEPES running buffer with 140 mM NaCl. MPIs based on a low molecular weight (10 kDa) core, MPI 6-MPI 9, exhibited slightly weaker binding and increased K_{d} when measured with most polyP chain lengths than those based on a 20 kDa core, MPI 1-MPI 5 and UHRA.

Differences were observed in binding between MPIs with different CBGs, with the smallest dissociation constants observed for the Me₆TREN ligand, with CBG II and CBG I showing slightly weaker binding affinity, albeit still sub-micromolar. The stronger binding of the Me₆TREN ligand may be explained by the higher pKₐ of the second and third protonation compared to the bare CBG I and CBG II ligands (Table 3), resulting in a higher charge density at physiological pH for Me₆TREN based UHRA (Table 2). The slightly increased binding strength of the CBG II based MPIs (MPI 4, MPI 5, MPI 8, and MPI 9) over those based on CBG I (MPI 1-MPI 3, MPI 6, and MPI 7) may therefore be attributed to the charge spacing on CBG II being more amenable to binding with the polyP anionic backbone. Furthermore, improved binding may be due to the increased charge density upon binding with CBG II due to the lower pKₐ of a second protonation event due to reduced strain induced by reduced electrostatic repulsion of the adjacent cationic amine residues when spaced by propyl linkers rather than the ethyl bridging groups used in CBG I.

### Example 6

Analysis of MPI Binding to polyP Via Isothermal Titration Calorimetry

Isothermal titration calorimetry (ITC) analyses were performed with MPIs of the present invention. The association constant (Ka) of the MPIs relative to different polyP chain lengths were measured. As well, the thermodynamic data of the binding reaction including the free energy (ΔG), the enthalpy (ΔH) and stoichiometry (N) of the binding reaction were obtained, as indicia of the driving force, strength and stability of the reaction (Table 5 and Table 6).

TABLE 5
Thermodynamic properties of binding, stoichiometry and binding affinity to polyP by
ITC, at pH 7.4 and 25° C.

| polyP^{a} | MPI ^{b} | N ^{c} | K_{d} (μM) | ΔG (kcal/mol) | ΔH (kcal/mol) | TΔS (kcal/mol) |
|---|---|---|---|---|---|---|
| P45^{d} | MPI 3 | 0.7 | 0.737 ± 0.008 | −8.37 ± 0.04 | −107.0 ± 0.8 | −98.7 ± 0.8 |
| | MPI 5 | 0.8 | 0.95 ± 0.05 | −8.22 ± 0.02 | −75.0 ± 0.4 | −66.8 ± 0.4 |
| | MPI 7 | 1.0 | 1.27 ± 0.09 | −8.05 ± 0.06 | −37 ± 1 | −29.0 ± 0.9 |
| | MPI 9 | 1.1 | 1.2 ± 0.2 | −8.08 ± 0.02 | −50.9 ± 0.5 | −42.9 ± 0.5 |
| P75^{d} | MPI 1 | 1.5 | 1.5 ± 0.3 | −7.9 ± 0.1 | −75 ± 2 | −67 ± 3 |
| | MPI 3 | 1.5 | 0.69 ± 0.04 | −8.41 ± 0.04 | −89.8 ± 0.4 | −81.4 ± 0.4 |
| | MPI 5 | 1.8 | 0.96 ± 0.09 | −8.05 ± 0.09 | −79.2 ± 0.9 | −71.1 ± 0.5 |
| P700^{e} | MPI 3 | 14.1 | 0.54 ± 0.04 | −8.56 ± 0.04 | −52.4 ± 0.7 | −43.8 ± 0.7 |
| | CBG I | 0.004 | (9 ± 2) × 10³ | −2.8 ± 0.1 | (−3 ± 2) × 10⁴ | (−3 ± 2) × 10⁴ |

^{a}Used in the ITC cell.
^{b} Added to cell via syringe.
^{c} Ratio of MPI to polyP.
^{d}Buffer used was sodium phosphate buffer composed of dibasic phosphate buffer (Na₂HPO₄), monobasic phosphate buffer (NaH₂PO₄) and NaCl. NaCl concentration is 10 mM.
^{e}Buffer used was HEPES buffer consisted of HEPES (4-(2-hydroxyethyl)-1-piperazineethanesulfonic acid) and NaCl.

TABLE 6
Thermodynamic properties of binding, stoichiometry and binding affinity
to polyP by isothermal titration calorimetry (ITC), at pH 7.4 and 25° C.

| | | Cell | Syringe | | | | | |
|---|---|---|---|---|---|---|---|---|
| | | (Binding | (Binding | | | | | |
| | NaCl | partner | partner | | | ΔG | ΔH | TΔS |
| Buffer | (mM) | 1) | 2) | N | K_{d} (μM) | (kcal/mol) | (kcal/mol) | (kcal/mol) |
| PBS^{a} | 150 | P75 | UHRA-8 | 1.2 | 1.71 ± 0.02 | −7.87 ± 0.01 | −127.7 ± 0.8 | −119.8 ± 0.8 |
| Sodium | 10 | P75 | UHRA-8 | 1.5 | 0.30 ± 0.08 | −8.91 ± 0.05 | −102 ± 1 | −93 ± 1 |
| phosphate^{b} | | | | | | | | |
| HEPES^{c} | 10 | P45 | CBG I | 5.1 | (7 ± 2) × 10³ | −2.9 ± 0.5 | −2 ± 2 | 1 ± 2 |
| HEPES^{c} | 10 | P45 | MPI 9 | 1.1 | 0.49 ± 0.03 | −8.66 ± 0.04 | −37.9 ± 0.9 | −29.2 ± 0.9 |
| HEPES^{c} | 10 | P45 | MPI 3 | 0.7 | 0.60 ± 0.09 | −8.50 ± 0.08 | −74.8 ± 0.8 | −66.3 ± 0.4 |

^{a}Phosphate buffered saline (PBS): 10 mM dibasic phosphate buffer (Na₂HPO₄), 2.7 mM KCl and 137 mM NaCl.
^{b}Sodium phosphate buffer: dibasic phosphate buffer (Na₂HPO₄), monobasic phosphate buffer (NaH₂PO₄) and NaCl.
^{c}HEPES buffer consisted of HEPES (4-(2-hydroxyethyl)-1-piperazineethanesulfonic acid) and NaCl.

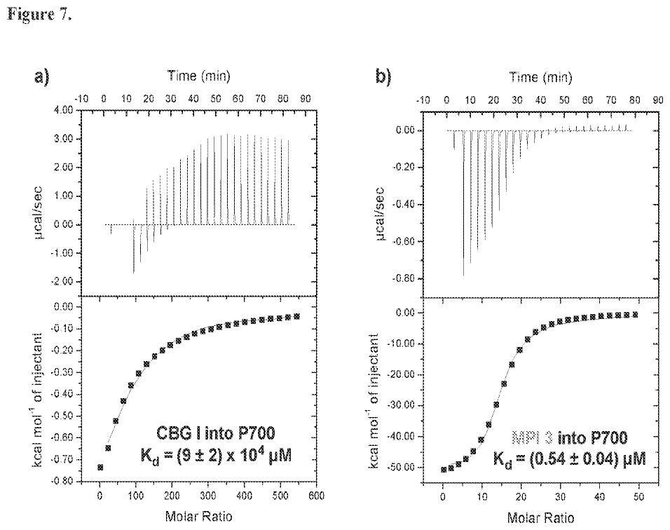

Titration ITC curves and binding curves for small molecule CBG and MPI 3 with LC polyP are provided in FIG. 7. Compared to its small molecule CBG I (PMDTA), MPI 3 exhibits strong binding with a binding affinity in the micromolar range. CBG I, however, exhibits a weak binding with a binding affinity in the millimolar range, indicating that the multivalent presentation of CBG I on MPI 3 plays a strong role in increasing its binding affinity to polyP (FIG. 7). While the binding interaction of each small molecule with polyP may be readily reversed such as with CBG I, the cumulation of many binding interactions present on MPI at the same time discourages the large molecule from diffusing away, ensuring the stability of MPI and polyP as a complex.

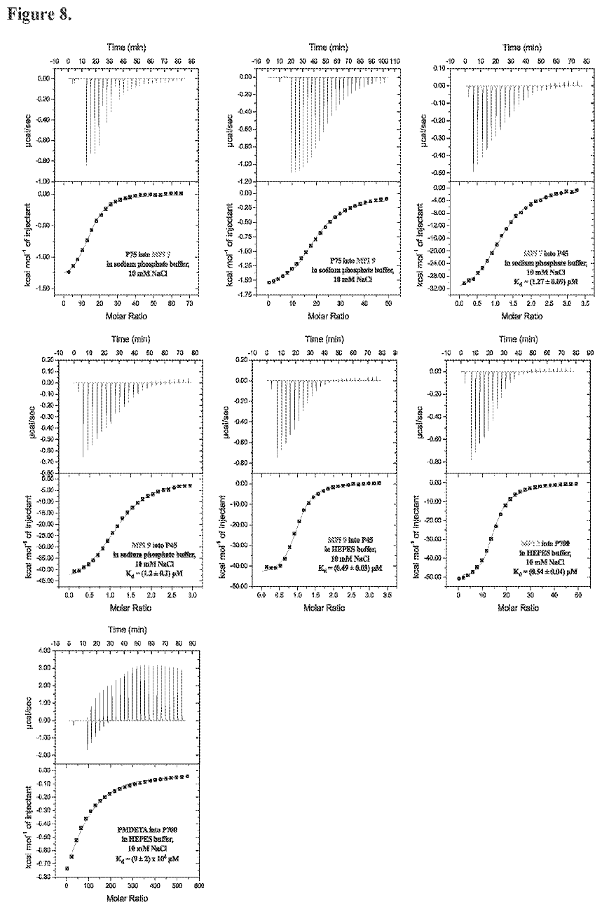

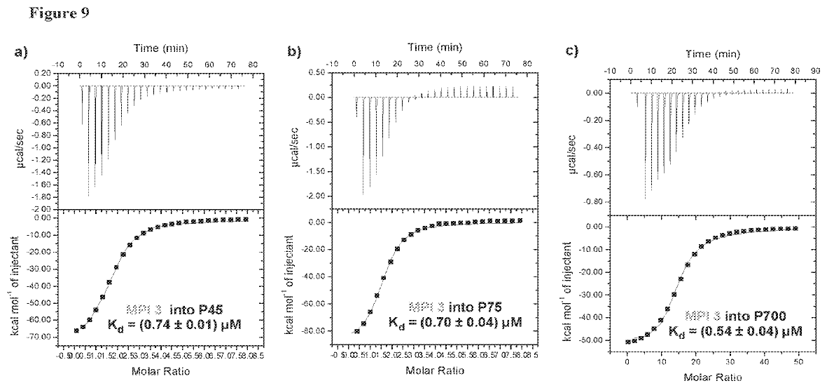

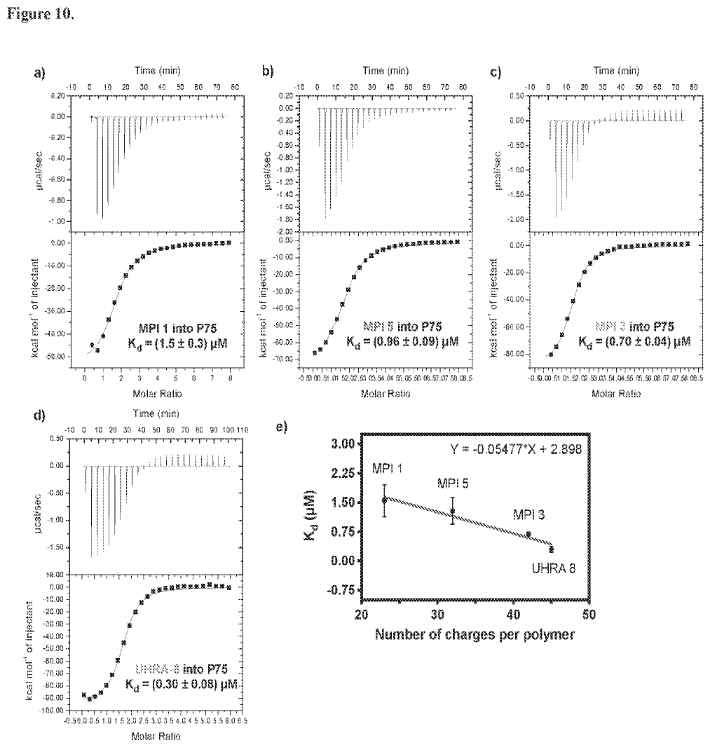

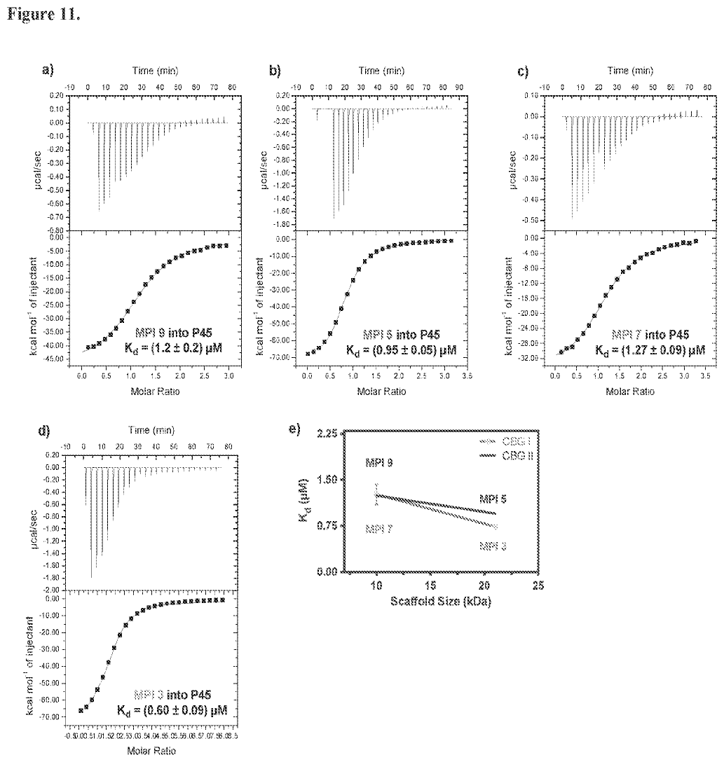

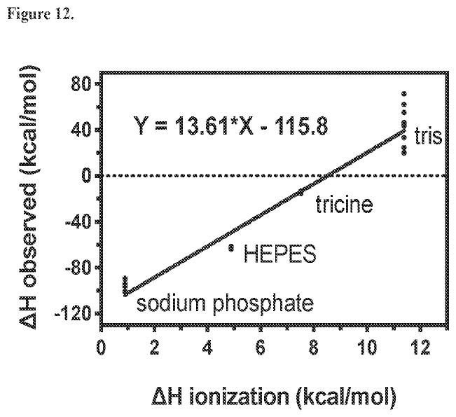

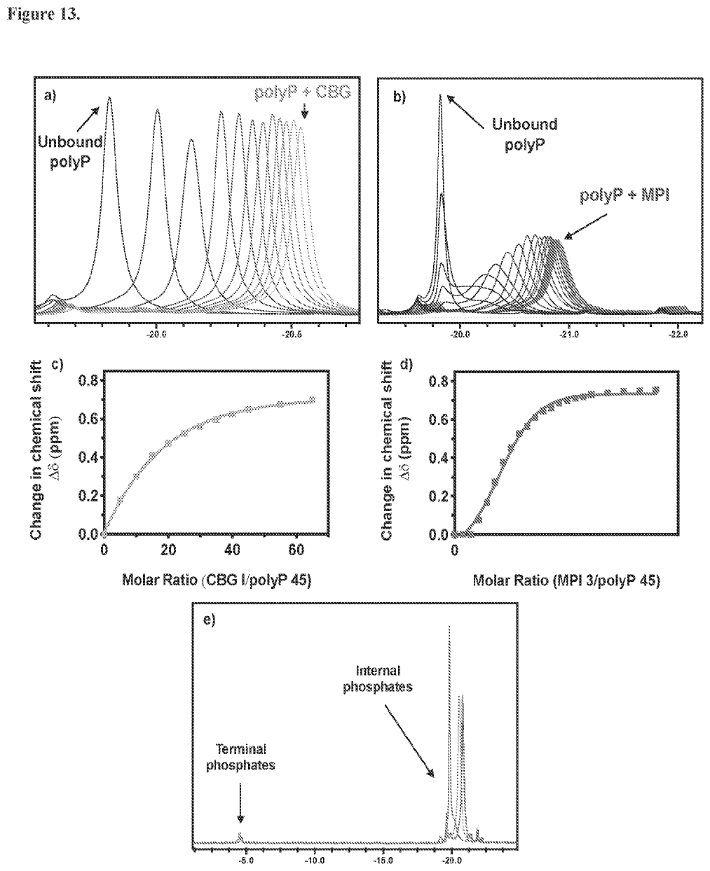

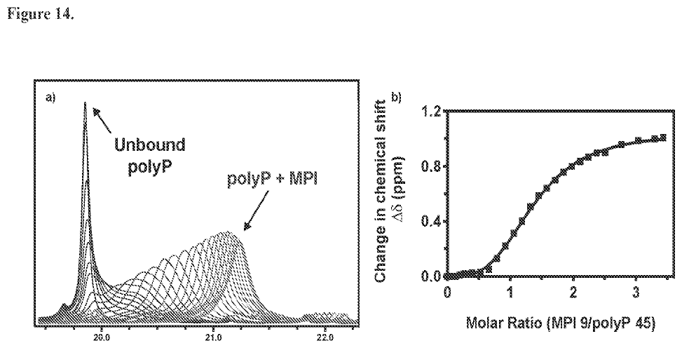

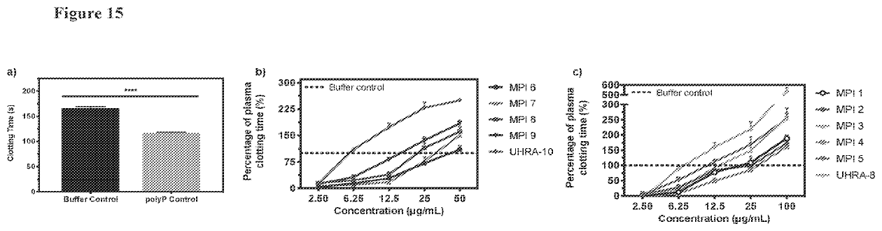

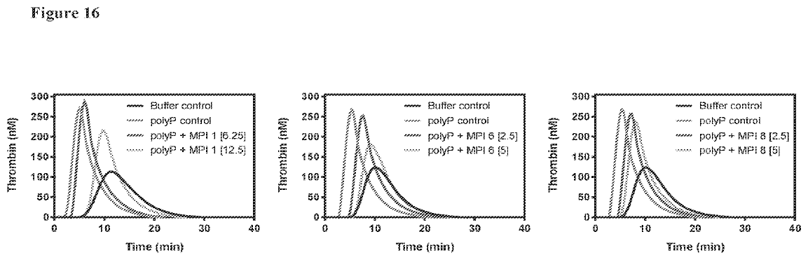

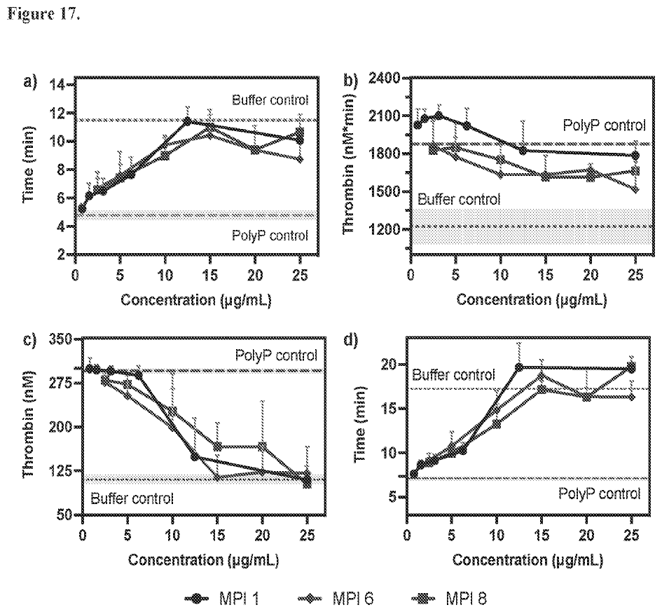

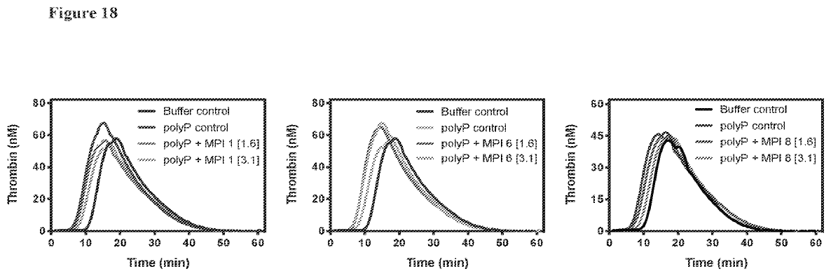

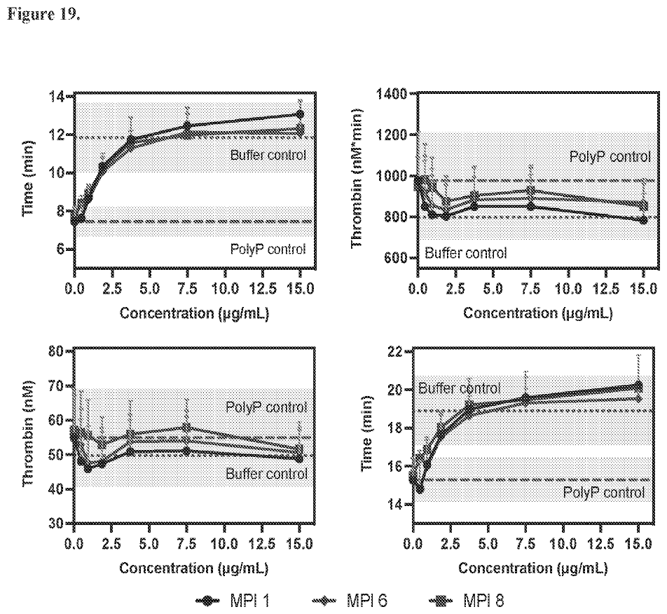

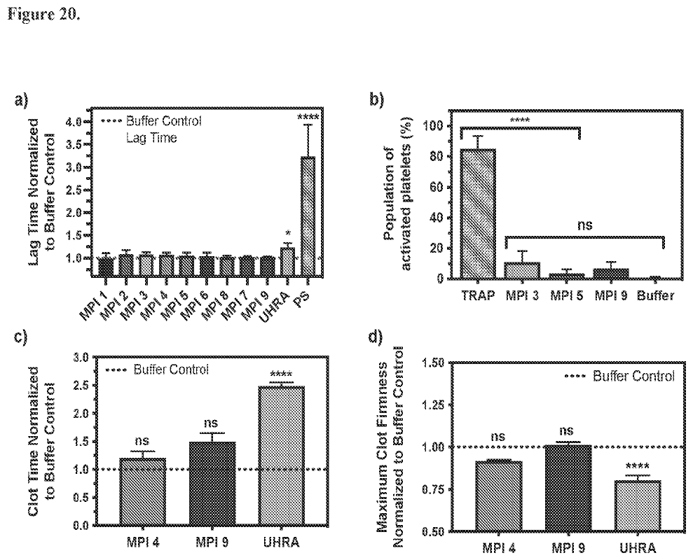

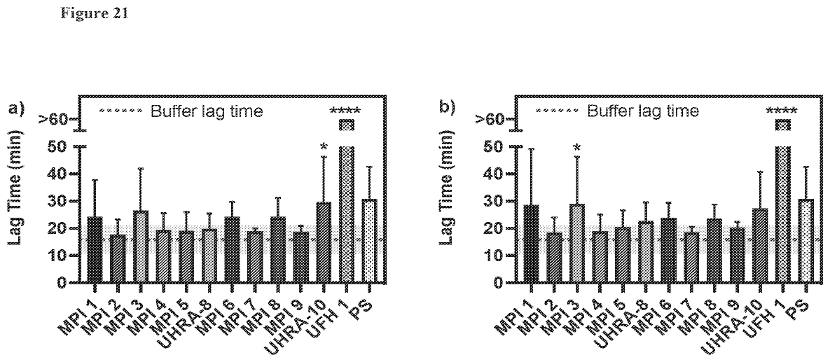

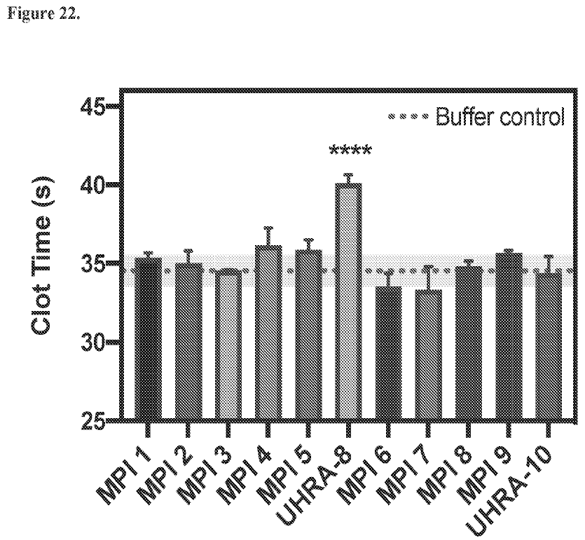

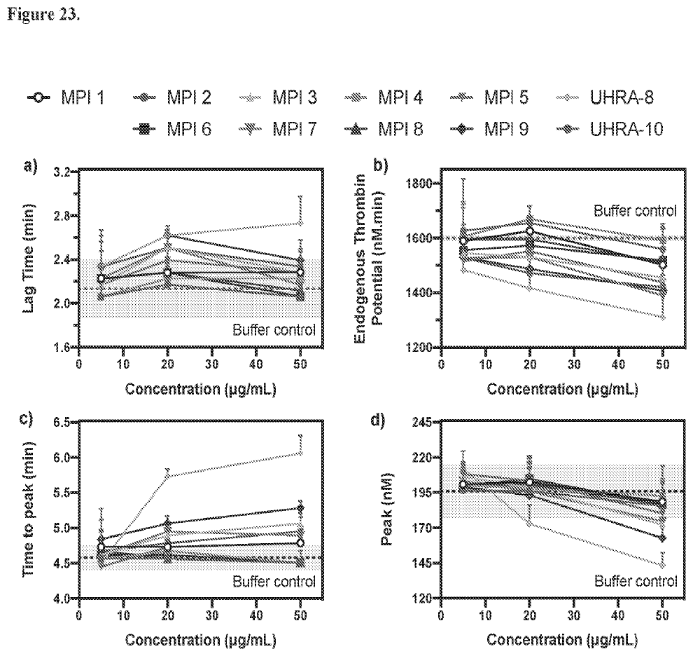

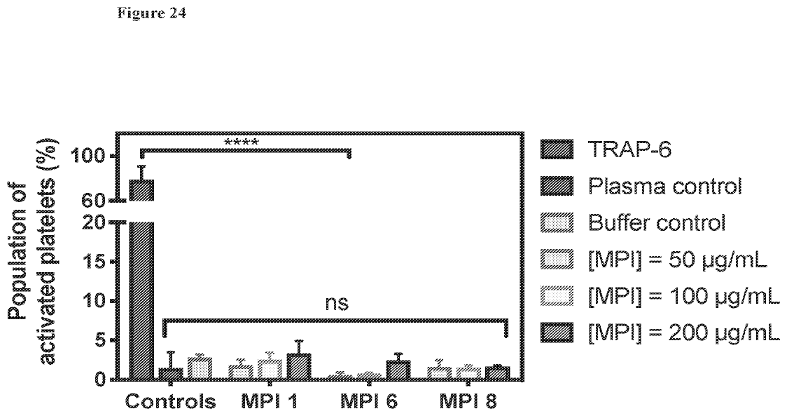

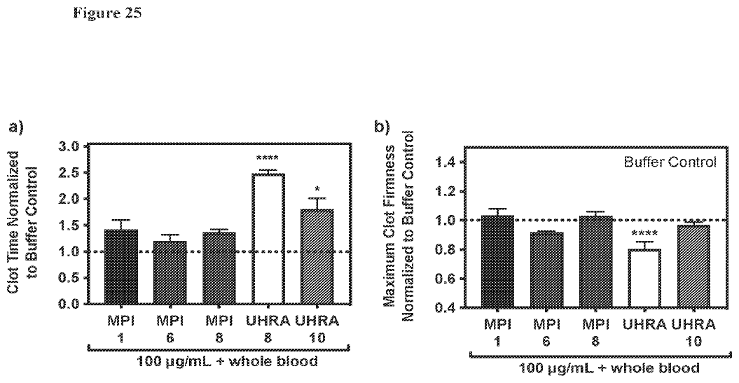

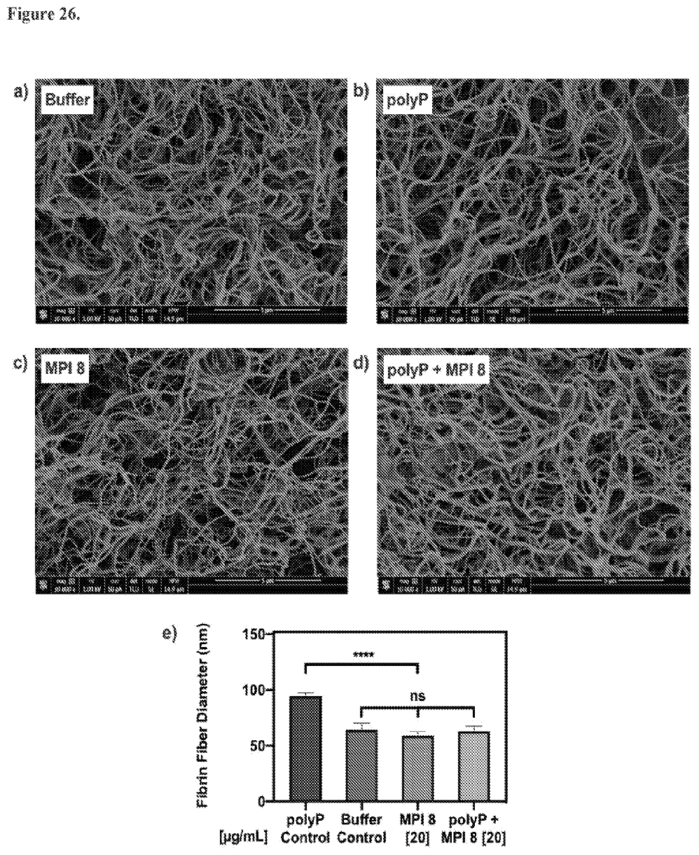

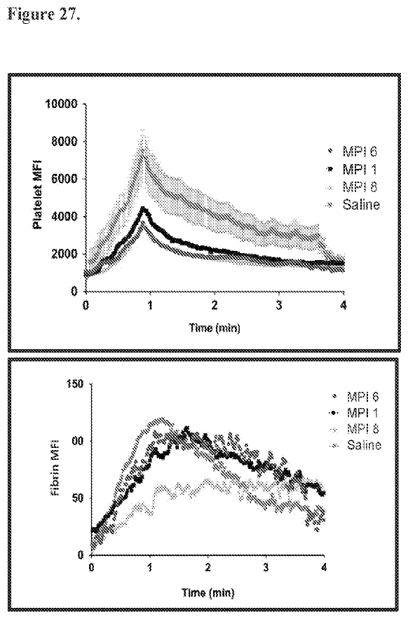

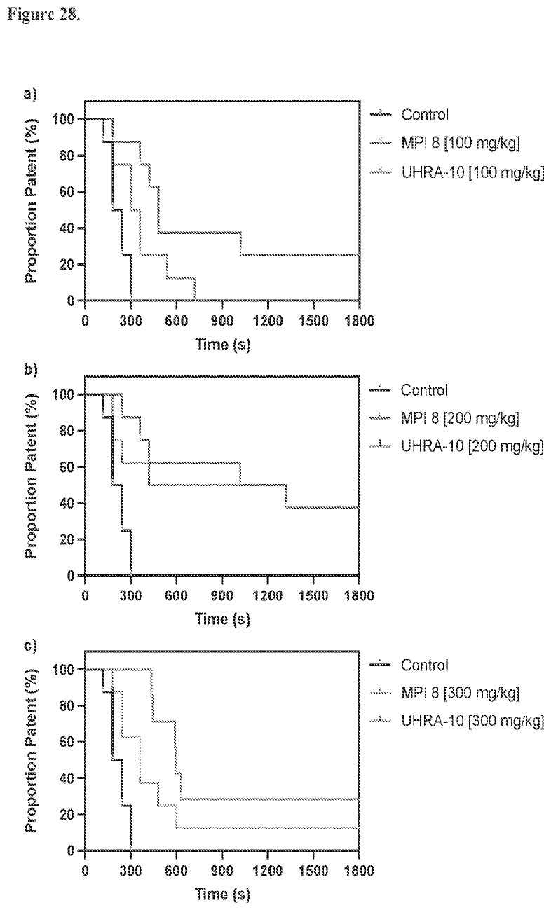

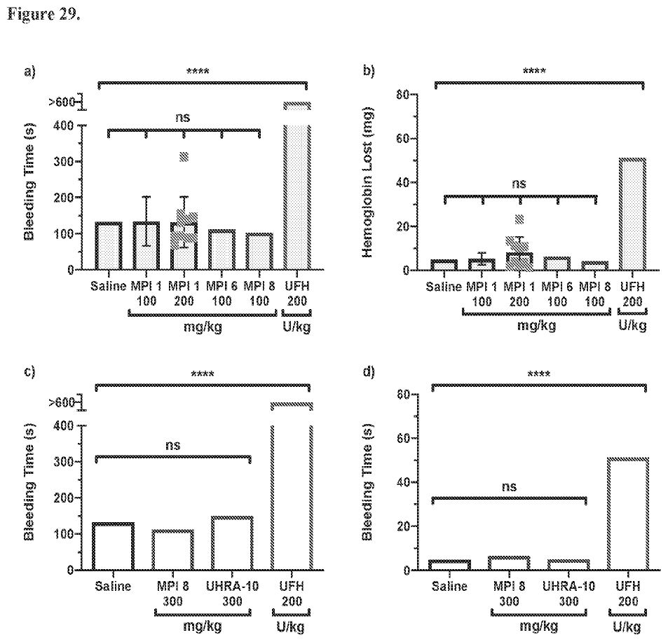

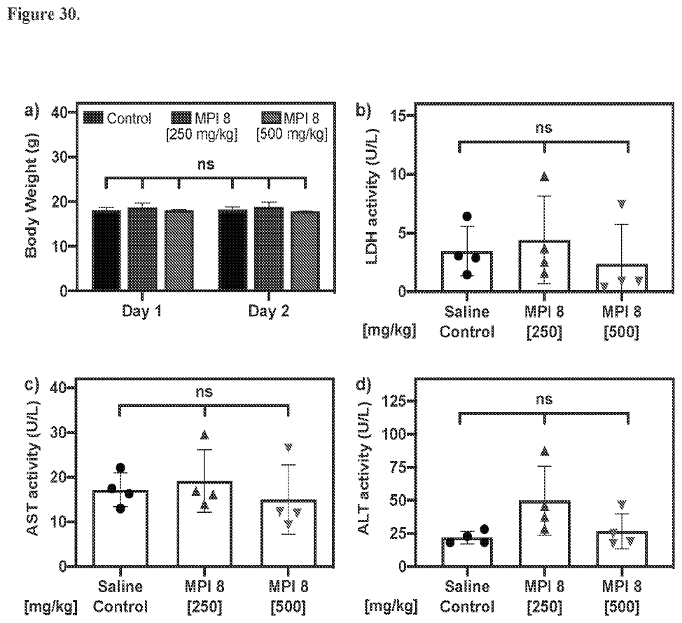

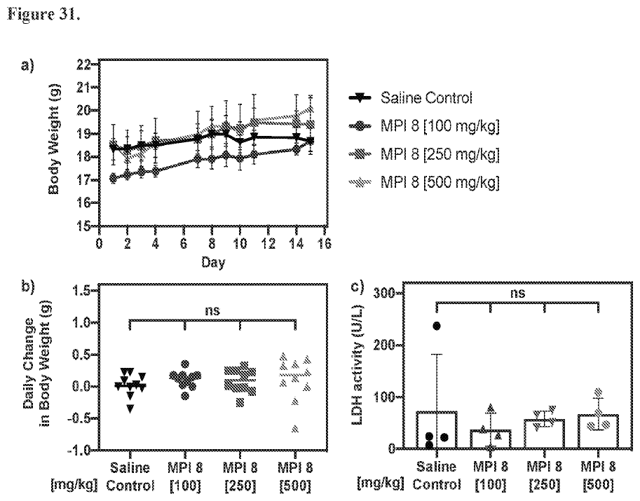

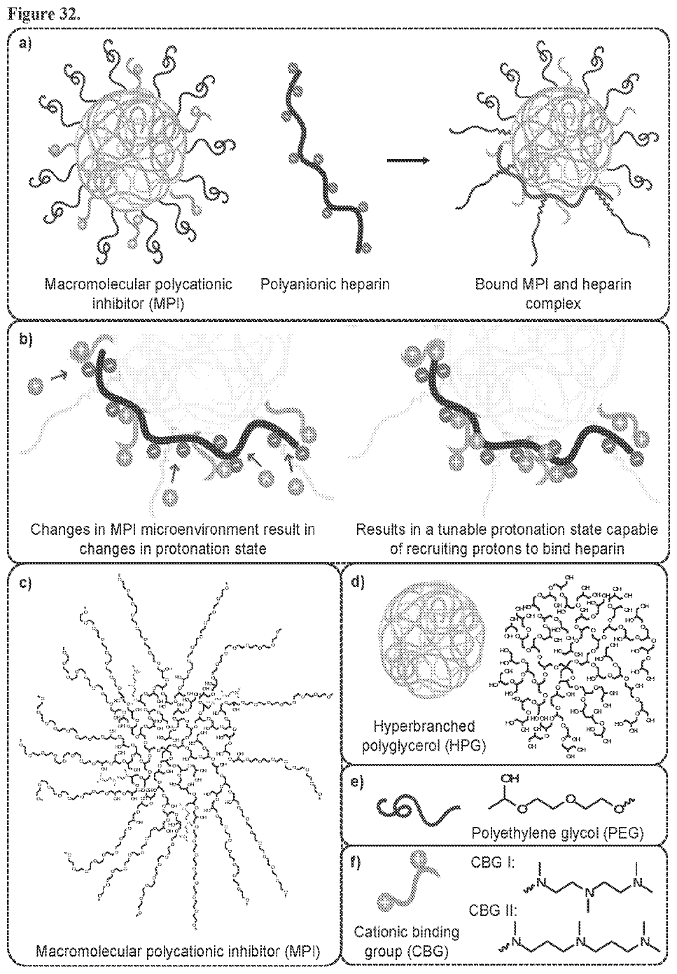

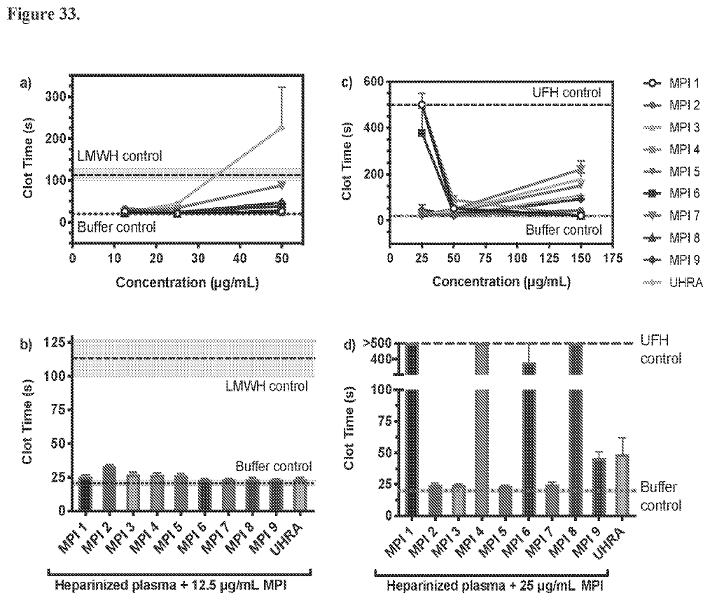

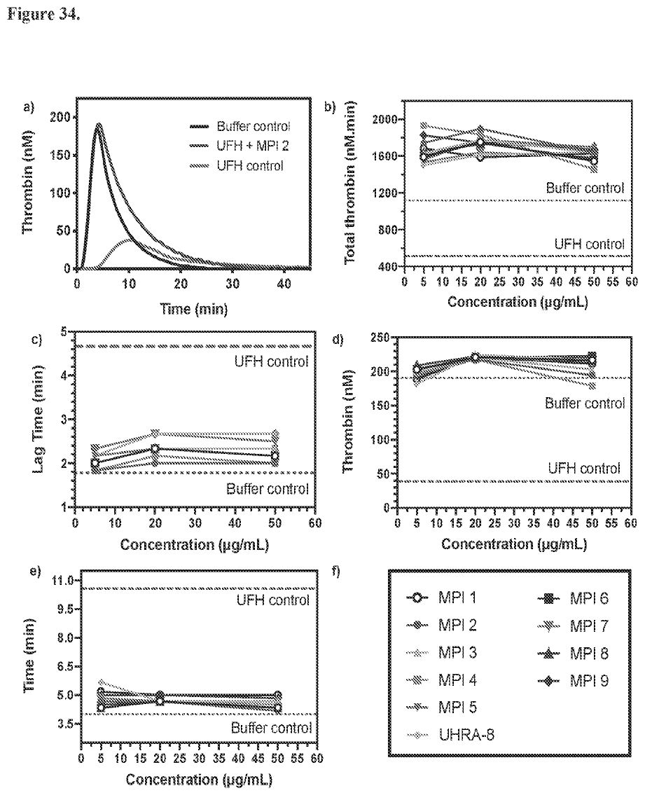

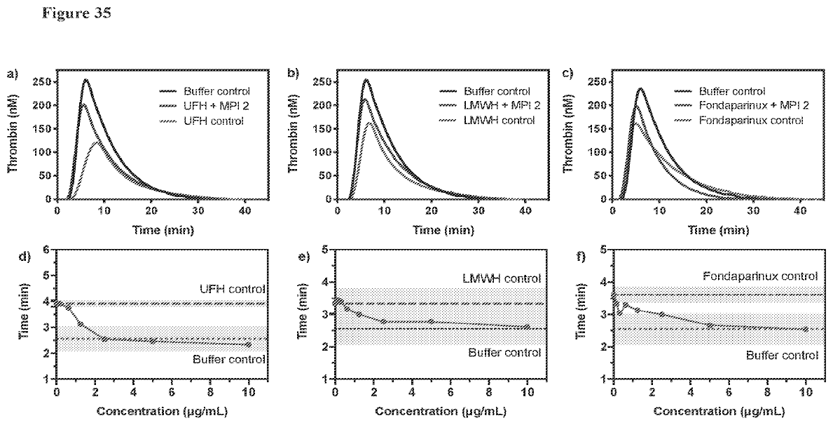

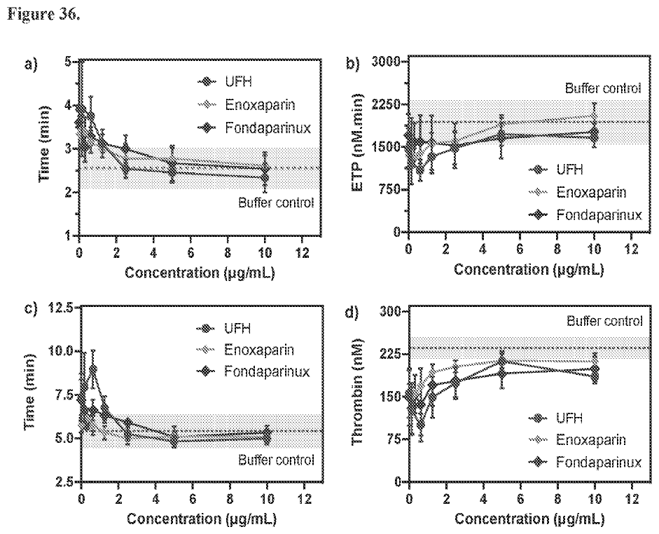

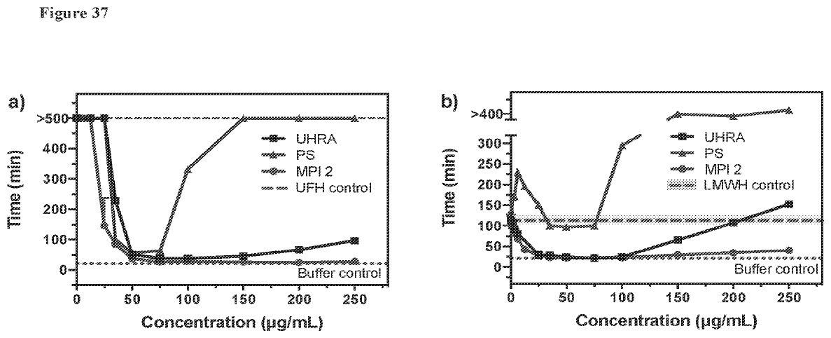

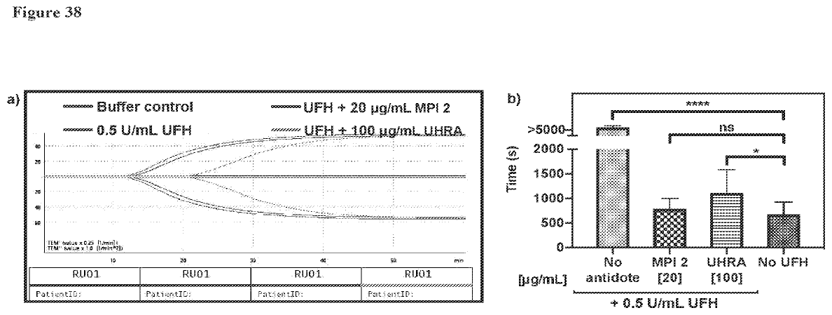

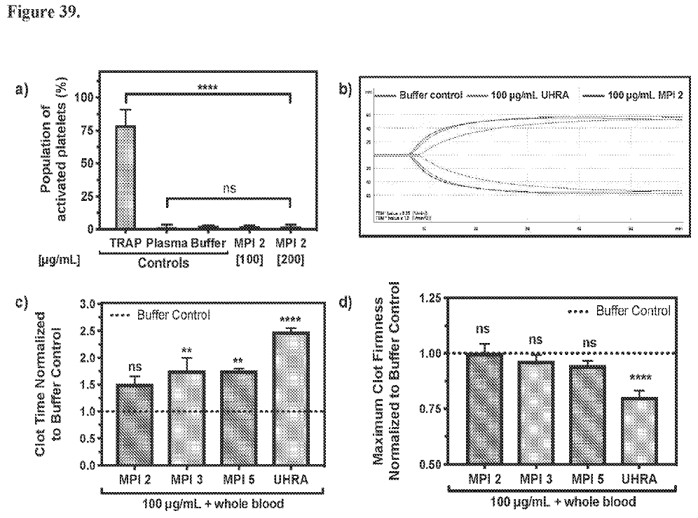

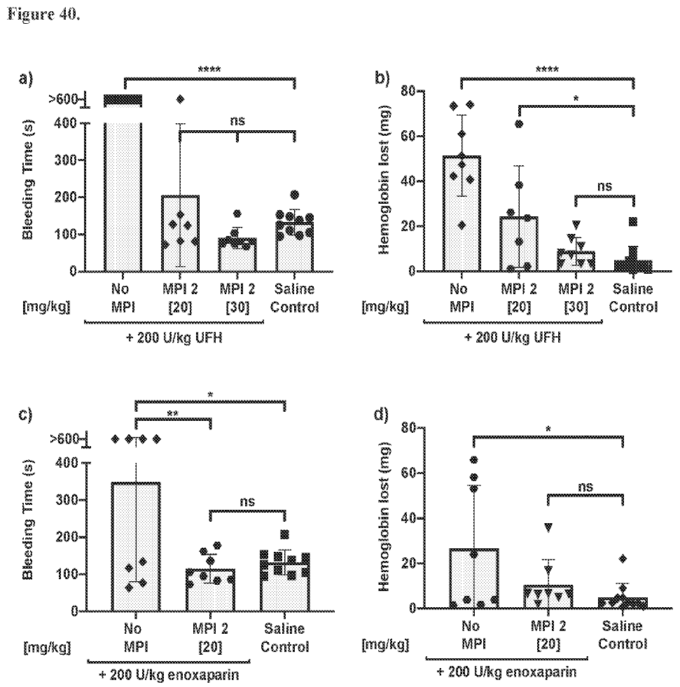

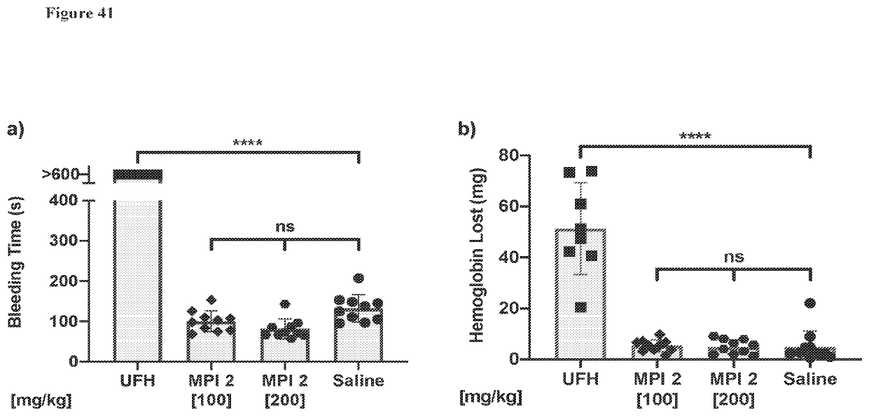

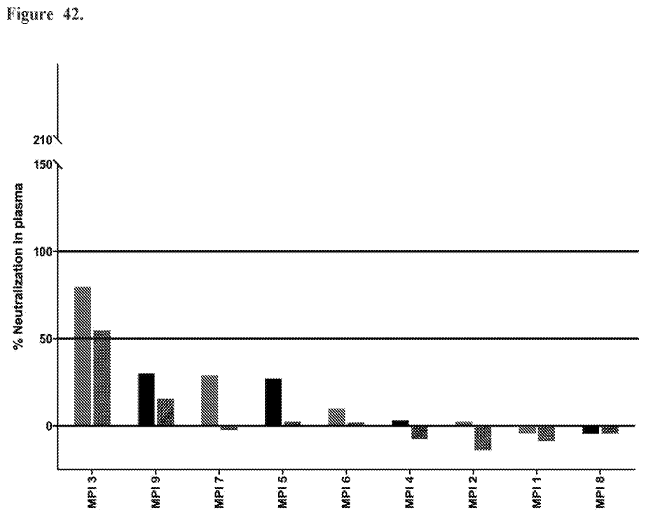

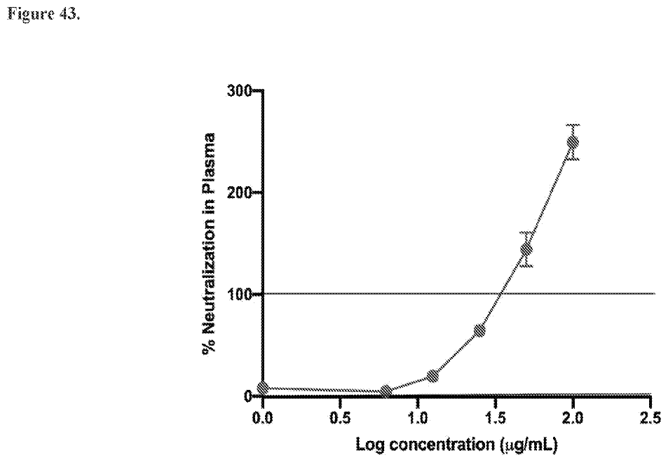

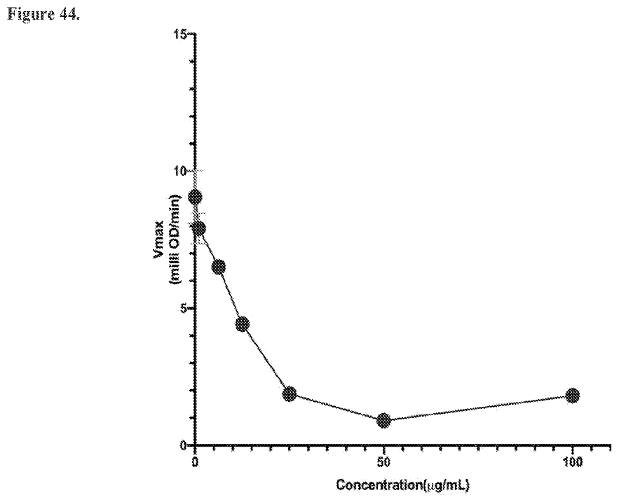

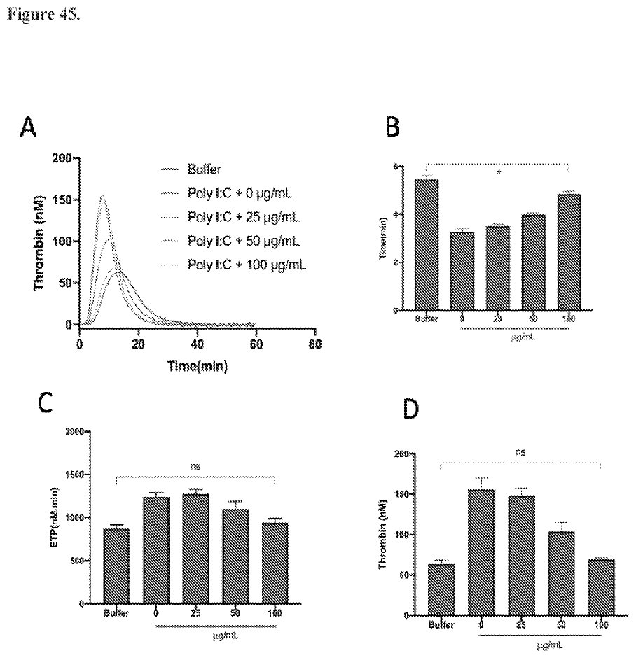

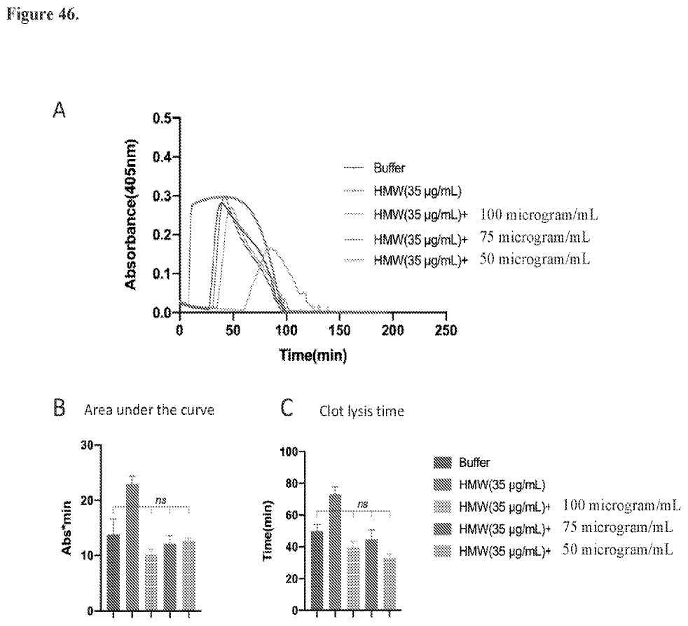

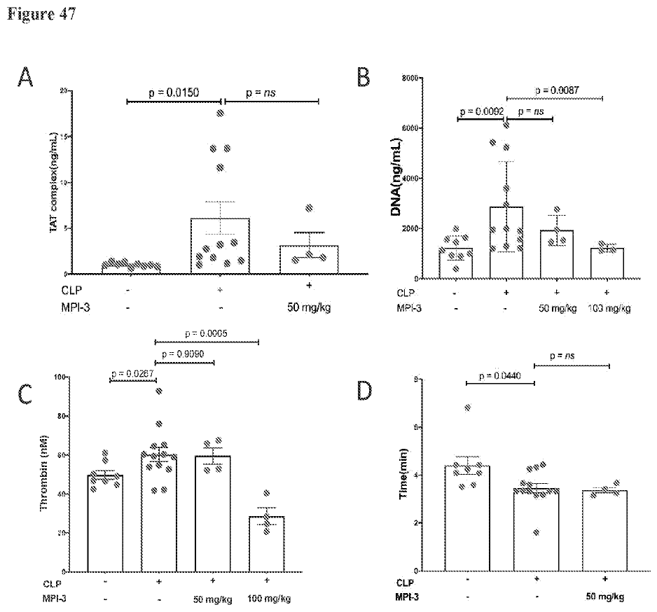

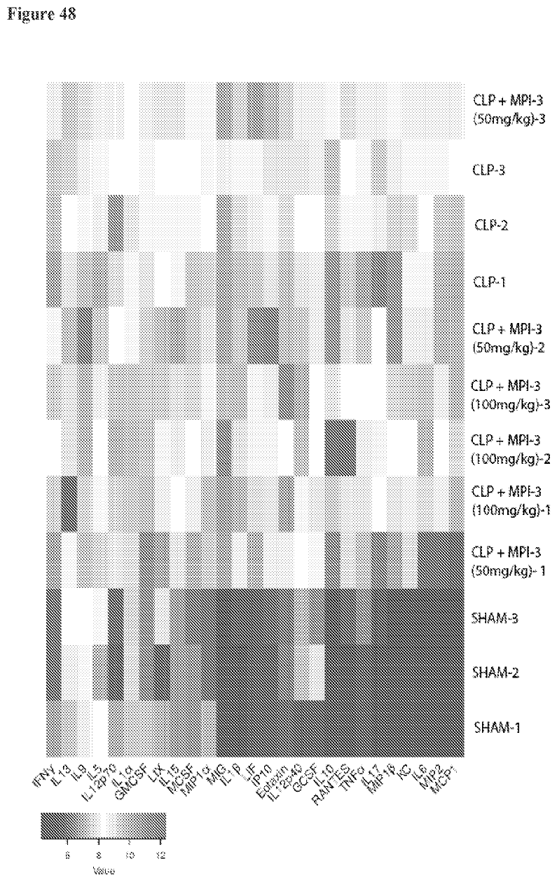

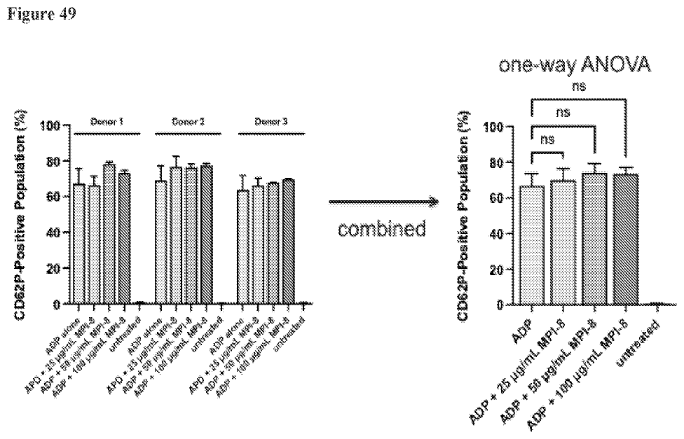

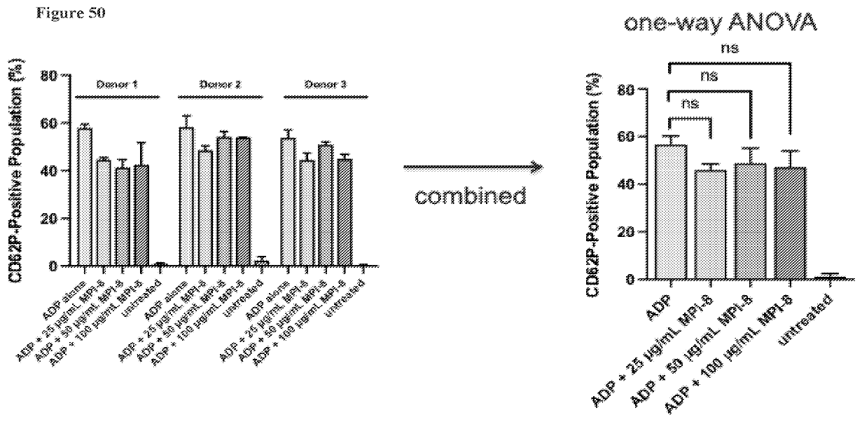

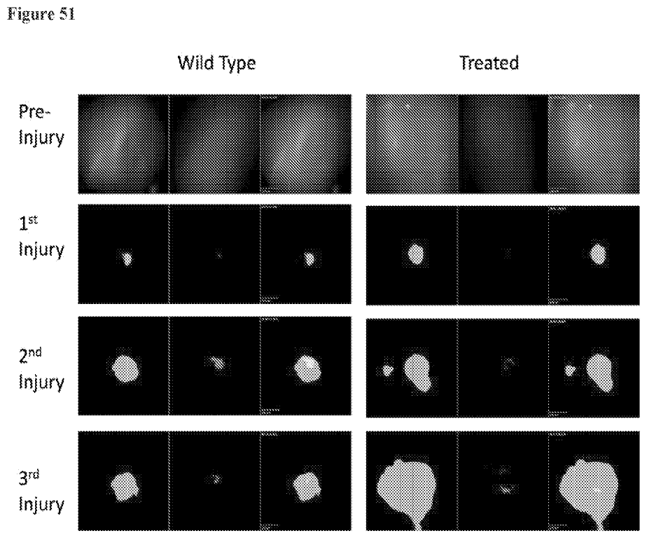

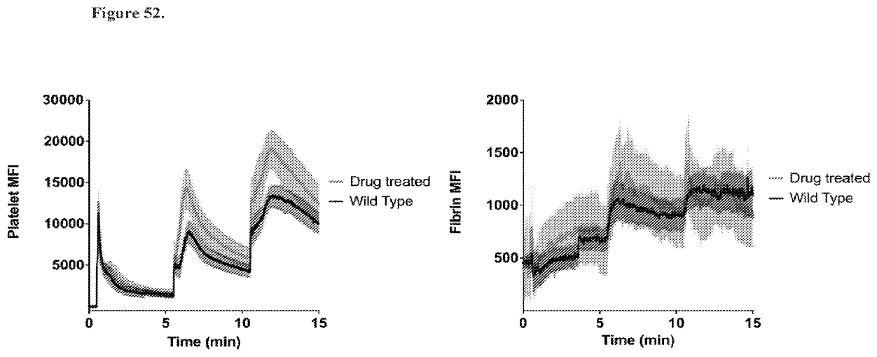

The binding affinities of MPI 3 toward different chain lengths of polyP (P45, P75, P700) (Table 5, Table 6) do not significantly change, indicating relative insensitivity of the MPI to the size of the polyP. Representative titrations for all binding pairs studied are shown in FIG. 8. A comparison of binding activity of MPIs with different CBGs was performed. We selected MPI 5, which exhibits comparable charge density at physiological pH as that of MPI 3. There was a larger enthalpy of binding for MPI 3 containing CBG I with two carbon spacer (ethyl spacer) between the amine than CBG II spaced with three 3 carbons propyl spacer (MPI 5). This is directly correlated to the higher pKₐ of the second protonation state of MPI 3 compared to MPI 5, as shown in Table 3.

When comparing MPI 1 and MPI 3, two MPIs with the same size scaffold and same CBG structure but different in the number of CBGs attached, apparent binding affinity increased with an increase in charge. This indicates that not all charges present on the MPI cores may be necessary for the binding to polyP. The binding affinities of MPI 3 toward different chain lengths of polyP (P45, P75, P700) (FIG. 9, Table 5, 6) do not significantly change, indicating the relative insensitivity of the MPI to the size of the polyP. As shown in FIG. 10, a correlation between the number of positive charges and apparent binding affinity was observed. Based on the slope obtained when plotting observed dissociation constants versus number of positive charges per polymer (FIG. 10e), each additional positive charge contributed to decreasing the dissociation constant by only 0.055±0.009 μM, indicating that charge density may be among many factors affecting the overall binding affinity. The dependence of binding affinity on cationic charge falls within the standard deviation of apparent binding affinities observed for the MPI library.

A comparison of MPI 9 and MPI 5 (FIG. 11 a-b) shows the effect of scaffold size on K_{d}. The 2 MPIs have the same CBG structure but different scaffold sizes and a relatively similar ratio in the number of CBGs attached per kDa of polymer. As can be seen from FIG. 11e, the experimental dissociation constant decreased slightly with increasing scaffold size. A similar trend was observed when comparing MPI 7 with MPI 3 (FIG. 11c-d). The smaller (10 kDa) polymer scaffolds provide a larger difference in size between the MPI and polyP resulting in a slightly less favorable assembly compared to the 20 kDa scaffolds. When comparing another set of MPI built on the same scaffold, MPI 7 and MPI 9, both built on the same 10 kDa polymer with ca. 15 CBGs per polymer as shown in FIG. 11, the apparent binding affinities were alike despite one having a higher degree of protonation in these conditions. This indicates that not all charges present on the MPI may be necessary for binding to polyP.

### Example 7

### Switchable Protonation Behavior of MPIs

In experiments conducted in the development of the present invention, we investigated the switchable protonation properties of MPIs using ITC experiments. The ITC analysis showed that even though MPIs have lower charge at physiological pH, comparable binding affinities are observed when compared to the higher charge density UHRA due to the switchable protonation states associated with our CBGs of the present invention on binding to polyP. This results in a higher cationic charge density for MPI upon binding to polyP. In the selected CBGs, a second protonation is possible with pKₐ of 6-7.

Potentiometric titration analyses demonstrate that the CBGs of the present invention exhibit slightly more than one positive charge per ligand at physiological pH, indicating that once MPI is partially bound to the polyanionic partner, the local dielectric conditions surrounding the binding site due to the presence of a high density of negative charges stabilize an additional CBG protonation. This causes in an increased population of doubly protonated CBGs, whose additional proton is acquired via proton recruitment from the aqueous surroundings, thereby resulting in higher cationic charge density on the MPI on polyP binding, and stabilizing the multidentate binding reaction without requiring a highly cationic conformation at physiological pH.

To confirm the switchable protonation ability of the generated MPIs, a series of ITC measurements were conducted using several buffers with a range of values for ΔH_{ion,buffer}⁰. If proton recruitment plays a significant role in the binding process, the effect of the heat of ionization of the buffer will be observed as a change in the observed ΔHₑₓₚ⁰. Therefore, to confirm the role of proton recruitment in the MPI-polyP binding process, the observed ΔHₑₓₚ⁰ was measured in different buffers and plotted against the ΔHᵢₒₙ⁰ on of each buffer. (Goldberg, R. et al. Thermodynamic Quantities for the Ionization Reactions of Buffers. J. Phys. Chem. Ref Data 2002, 31 (2), 231-370.) (FIG. 12). Using Equation 2, we estimated the number of protons involved in the recruitment process for MPI 3, by measuring the slope of the resulting plot. In a less complex binding process, such as a ligand-protein binding event, this slope can be directly correlated to the number of charges recruited in the binding process. (Neeb, ibid.) In the case of the MPI-polyP binding process, however, the dispersity of both polymeric binding partners provides a challenge to extract an exact value for the number of protons recruited. The line of best fit, plotted in FIG. 12, indicates that approximately 14 protons are recruited when MPI 3 binds to P75, but the errors on this measurement give a 95% confidence interval on this slope from 7 to 20 protons recruited per binding event. Because MPI 3 is composed of approximately 31 CBGs with a charge density of 42 charges per MPI at pH 7.4, this indicates a significant increase in the number of positive charges on MPI, with each CBG recruiting between 0.23 and 0.65 protons per ligand resulting in a 16 to 48% increase in cationic charges upon binding. The data indicate that during the binding process between MPI and polyP, a significant increase in the cationic density of the MPI is observed consistent with binding in which bound MPI exhibits significantly higher charge density upon binding than when it is at physiological pH. The initial multivalent binding of the MPI is strong enough such that the protonatable sites on MPI with pKₐ values 6-7 overcome the energy barrier of recruiting a proton from the surroundings and satisfy the overall charge requirement to strongly bind polyP. Higher charge density polyanions such as polyP overcome this initial energy barrier resulting in selective binding to polyanions with higher charge density that match the charge map of the MPI, in contrast to the situation resulting in the poor binding to low charge density polyanions such as proteins and cell surface. (Kalathottukaren and Abbina, ibid.)

$$Δ {H}_{exp}^{0} = Δ {H}_{bind}^{0} + n Δ {H}_{ion , buffer}^{0} \qquad Eq . ( 2 )$$

### Example 8

Effects of MPI Binding on polyP Via ³¹P NMR

While ITC experiments indicate the behavior of the amine functionalities on the CBGs during the MPI-polyP binding event, the effect of the MPI on the polyP phosphate backbone have previously been unknown. To determine the interaction between the MPI and polyP backbone, and how the interaction effects the electronic environment of the phosphorus nuclei, ³¹P NMR titration experiments were performed. PolyP of 45 chain length was used for the measurement because for this molecule both internal and terminal phosphorus units may be visualized. In these experiments, the binding properties of small molecule CBGs were compared directly to the behavior of the same CBGs bound to an MPI (FIG. 13). While it is not possible to quantify the number of bound phosphate units with MPI or CBG, it is possible to visualize the change in electronic environment of the phosphorus nuclei and extrapolate the binding behavior between MPI and polyP.

Upon titrating polyP (P45) with MPI 3 and CBG I, a distinct binding behavior was observed. This titration showed only one major internal phosphate peak at each concentration of CBG I tested, with approximately equal intensity between the runs (FIG. 13a). This finding of a broad average peak for all internal phosphates in the polyP chain shifting with increasing concentration is indicative of rapid exchange of the phosphate-CBG complex formed on time scales much faster than the NMR observation window. This implies that the binding between this small molecule CBG and polyP is a weak interaction by nature, and that the weak interaction results in one broad peak shifting as additional CBG I is added to the titration, while the peak intensity is maintained.

The interaction between MPI 3 and P45 does not exhibit this behavior. The ³¹P NMR spectra obtained during the binding of polyP to MPI showed the phosphorus nuclei exists in two potential states, an unbound state and a bound state (FIGS. 13b and d). Upon addition of MPI 3, the volume of the peak arising from unbound polyP decreases indicating a decrease in unbound phosphates, while a peak arising from bound phosphates gives rise to a new bound peak. Because the phosphates that have bound MPI are tightly bound (strong interaction with MPI), they are not rapidly dissociating and associating with the P45 backbone and give distinct ³¹P signals for the bound and unbound internal phosphates.

These observations indicate that the multivalent nature of the MPI-polyP binding event results in a much stronger interaction than would be expected for just the small molecule CBG I alone, even with a much higher molar equivalent (millimolar range). This experiment was carried out with a representative 20 kDa MPI, MPI 3 with P45, as a direct comparison to CBG I, as well as additional experiments with MPI 3 and P700 and MPI 9 with P45 to demonstrate the consistency of this binding behavior over different core sizes, CBGs, and polyP binding partners (FIG. 14).

In addition, observing the electronic behavior of the phosphorus nuclei by ³¹P NMR while slowly titrating a consistent amount of MPI 3 enabled the visualization of the identity of phosphorus on the entire chain that would first bind to MPI. Upon the initial addition of MPI, a change in chemical shift was observed for the internal phosphorus groups, while the terminal phosphorus peaks remained unchanged (FIG. 13e). These data indicate a tendency for the internal phosphate groups to participate in the binding event, over the terminal groups attributed to the fact that the internal P-O are strongly acidic, while the terminal P-O are weakly acidic. (Lamm, O. and Malmgren, H. Dispersitatsmessungen an Einem Hochpolymeren Metaphosphat Nach Tammann. Z. Für Anorg. Allg. Chem. 1940, 245 (2), 103-120.)

### Example 9

Inhibition of Long Chain polyP Measured by Plasma Clot Time

Plasma clotting triggered by recalcification and LC polyP (P700) was used to investigate the inhibition activity of MPI candidates. LC polyP, UHRA-8 and buffer were used as controls. The ability of MPIs to inhibit the procoagulant effects of polyP is shown in FIG. 15. As can be seen, plasma with no polyP added (clotting was initiated by calcium only) gave an average clot time of 166±3 seconds, while the control with LC polyP had an average clot time of 117±1 seconds, demonstrating the procoagulant activity of LC polyP. Increasing concentrations of MPIs inhibited the procoagulant activity of polyP, as evidenced by the normalization of clotting time in comparison to the buffer control. Some MPIs, e.g., MPI 5 and MPI 9, normalized the clot time to that of buffer at concentrations as low as 12.5 μg/mL. Other candidates such as MPI 1, MPI 2 and MPI 3 showed lower activity at the same concentration. UHRA-8 demonstrated a prolongation of clot time at higher concentrations, with clot times greater than 550 seconds, while MPI candidates did not show this increase even at high concentrations of 100 μg/mL (FIG. 15).

### Example 10

Inhibition of Long Chain and Short Chain polyP Via Thrombin Generation Measurement

Thrombin generation was investigated in presence of LC and SC polyP using calibrated automated thrombography, a sensitive method for evaluating the effect of the added blood coagulation modulators. In this assay, a calibrated fluorogenic substrate is used to infer the quantity of thrombin generated. Addition of LC polyP, a potent activator of clotting, significantly shortens the clot time of plasma, while the addition of MPI normalized the thrombin generation curve (FIG. 16). Clotting parameters such as lag time, endogenous thrombin potential (the total amount of thrombin generated, quantified as the area under the curve), time to peak and peak thrombin were evaluated in these experiments (FIG. 17). Based on these data, we identified the novel MPI compounds of the present invention. The dose-dependent response of polyP inhibition activity was notable in the case of the lead MPI candidates (MPIs 1, 6, and 8) for which a nearly complete return of thrombin generation parameters (i.e., lag time, peak thrombin, and time to peak thrombin) to that of buffer control was observed. Together, these data show the inhibition activity of MPIs of the present invention against LC polyP.

The inhibition potential of MPI against SC polyP was investigated by measuring plasma clotting triggered by tissue factor (TF), using thrombin generation assay. While SC polyP (platelet sized polyP) is a less potent activator of the contact pathway, it plays many roles in the downstream events of clotting and remains a useful target to develop antithrombotic agents. Less pronounced effects are seen than in the case of LC polyP in thrombin generation; the short chain polyP remains a strong accelerant for blood clotting (FIG. 18).

A titration of the MPI candidate library provides a dose dependent response of short chain polyP inhibition, normalizing thrombin generation parameters to that of the buffer control (FIG. 19). MPIs 1, 6 and 8 demonstrate the normalization of lag time, endogenous thrombin potential, peak thrombin, and time to peak thrombin: values close to those observed for the buffer control. From the dose-response curves generated for each MPI compound, we calculated the half maximal inhibitory concentration (IC₅₀) as shown in Table 7.

TABLE 7
Half maximal inhibitory concentrations of MPI towards long chain
and short chain polyP via the thrombin generation assay.

| | IC₅₀ Long chain polyP | IC₅₀ Short chain polyP |
|---|---|---|
| | (μg/mL) | (μg/mL) |
| MPI 1 | 6.6 ± 0.2 | 1.9 ± 0.1 |
| MPI 2 | 11.5 ± 1.5 | 2.8 ± 0.4 |
| MPI 3 | 3.8 ± 0.5 | 1.7 ± 0.3 |
| MPI 4 | 14.3 ± 5.3 | 2.9 ± 0.5 |
| MPI 5 | 6.0 ± 2.0 | 1.6 ± 0.2 |
| UHRA-8 | N/A | 1.9 ± 0.5 |
| MPI 6 | 5.3 ± 2.1 | 1.7 ± 0.4 |
| MPI 7 | 5.6 ± 2.3 | 1.3 ± 0.1 |
| MPI 8 | 8.5 ± 2.9 | 1.8 ± 0.2 |
| MPI 9 | 7.6 ± 2.4 | 0.9 ± 0.1 |
| UHRA-10 | 11.9 ± 10.1 | 1.4 ± 0.1 |

While MPI compounds and UHRA-10 generated classical sigmoidal dose-response curves which could be fitted using the Hill equation, UHRA-8 did not fit this model. The effects of each MPI on plasma clotting triggered by LC polyP on 4 clotting parameters were assessed. Since LC polyP is a potent activator of the contact pathway, the lag time was taken as the primary parameter affected by the addition of LC polyP, and was used to generate the IC₅₀ of each MPI. This methodology was also used to determine the half maximal inhibitory concentration of each MPI towards SC polyP in this plasma clotting system. While a range of IC₅₀ values towards SC and LC polyP were observed for each MPI and UHRA compounds, all 20 kDa and 10 kDa MPI candidates demonstrated a lower IC₅₀ value towards LC polyP than UHRA-8 and UHRA-10 respectively (Table 7). Moreover, MPI compounds of the present invention possess significantly less charge than their UHRA (about half to two thirds lower), and no significant loss of inhibition efficacy against both LC and SC polyP was observed.

### Example 11

Biocompatibility of MPI Candidates—Low Charge State of MPI Resulted in High Compatibility with Blood Components and Blood Clotting In Vitro.

To characterize the hemocompatibility of the MPI library, the effect of the polycationic molecules on plasma clotting kinetics was tested in the absence of added polyP. Previous studies on polycations have shown that they adversely impact blood coagulation. (Malik, N. et al. Relationship between Structure and Biocompatibility in Vitro, and Preliminary Studies on the Biodistribution of 125I-Labelled Polyamidoamine Dendrimers in Vivo. J. Control. Release Off. J. Control. Release Soc. 2000, 65 (1-2), 133-148., and Moreau, ibid.) Additionally, we investigated the effect of MPI candidates on whole blood coagulation, platelet activation and their influence on clot structure. These in vitro studies together with polyP inhibition activity allowed us to identify lead candidates for further investigation in vivo using mouse models.

To demonstrate the utility of the charge-switchable MPIs over previously reported polyP inhibitors, the biocompatibility and activity in vitro of these MPIs were characterized. The blood compatibility of the MPIs were measured using blood clotting analysis using pooled normal human plasma of each MPI relative to a buffer control (FIG. 20). Significantly increased or decreased lag time in this analysis in comparison to buffer control implies that they alter blood clotting and could potentially interfere with hemostasis. (Kalathottukaren and Abraham, ibid.) Up to 200 μg/mL of MPI did not significantly prolong plasma clot time compared to the buffer control. However, even 75 μg/mL of UHRA did prolong plasma clot time (FIG. 20a). Polycationic therapeutics such as protamine sulfate (PS) performed significantly worse than MPI candidates.

Further advantages of the low charge state of MPIs at physiological conditions were evidenced by their compatibility with blood components, such as platelets. Platelets can be activated by polycations such as PEI and PAMAM dendrimers which cause clotting complications and affect normal hemostasis. (Jones, ibid.) Platelet activation in presence of MPI in comparison to other controls are shown in FIG. 20b. Even at high concentrations of 100 μg/mL, the highest charged MPI candidates of the library did not induce a significant amount of platelet activation compared to that of the buffer control.

Additional assessment of the influence of MPIs on the clotting characteristics of human whole blood containing all of its components demonstrated no significant effect on the clot time or maximum clot firmness compared to the negative control with no MPI added (FIGS. 20c and 20d). On the other hand, UHRA at the same concentration of MPI tested at 100 μg/mL did significantly prolong the clot time and minimize the maximum clot firmness.

### Example 12

### MPI Influence in a Recalcification Triggered Plasma Clotting System

The clotting properties of citrated pooled PPP were assessed in a turbidity microplate assay in the presence of two different concentrations (75 and 200 μg/mL) of MPIs. Clotting was initiated by recalcification and the time taken to form a turbid clot from absorbance measurement was taken as the lag time. (Carter A. M. et al. Heritability of Clot Formation, Morphology, and Lysis. Arterioscler. Thromb. Vasc. Biol. 2007, 27 (12), 2783-2789.) We used UHRA-10, UHRA-8, UFH, protamine and buffer as controls added to PPP. Any significant deviation in lag time by the MPI candidates with respect to buffer control is considered as a change in clotting profile. As shown in FIG. 21, results indicate that most MPIs did not significantly change the lag time, even when measured at concentrations as high as 200 μg/mL. Previous polyP inhibitor, UHRA-10, and polycationic polypeptide protamine showed an increase in lag time compared to MPI candidates at 75 μg/mL. These results demonstrate that most MPIs do not affect plasma coagulation at the conditions studied.

### Example 13

### MPI Influence on Clot Time in a TF-Initiated Plasma Clotting System

We investigated the influence of MPI on clotting in a TF-initiated system measured on a coagulometer using FXII deficient plasma. In addition to MPI candidates, control polyP inhibitors and buffer control were evaluated. Results are shown in FIG. 22. In all cases except UHRA-8 there was no significant difference between the clot time with MPI and the buffer control, implying that MPI did not cause any significant changes to clot time in this TF-triggered clotting system. These data indicate that the prolongation of clot time observed when clotting is triggered by polyP is directly a function of inhibition of polyP, and not due to non-specific downstream effects on other steps in the coagulation cascade. Improvements over UHRA-8 highlight the improved biocompatibility of this new generation of MPI compounds of the present invention.

### Example 14

MPI influence on thrombin generation in TF-initiated plasma clotting

In view of the minimal effect of MPIs on clot time in the coagulation assays described, we further characterized the influence of the MPI library on the clotting process. This was performed utilizing a calibrated automated thrombography analysis, e.g., a thrombin generation assay (TGA). This assay provides enhanced sensitivity to thrombin generation, providing more insight into the effect MPI has on the clotting process by allowing for measurement of the quantity of thrombin generated over time compared to other methods. This assay also provides a direct method to measure additional parameters including endogenous thrombin potential, time to peak thrombin, and peak thrombin concentration. FIG. 23 shows data generated from these studies. The shaded area in the figure panels represent the buffer control. As shown, there are improvements are associated with the different MPI candidates on various TGA parameters compared to conventional polyP inhibitors (UHRA-10 and UHRA-8) with increasing concentration. While most MPI compounds do not cause variation in TGA parameters compared to buffer control at low concentrations (10-20 μg/mL), certain compounds showed large changes in thrombin generation behavior at high concentrations (most notably MPI 3 and MPI 9). Select MPI compounds (MPI 1, MPI 2, MPI 4, MPI 6, MPI 8) demonstrated no influence on TGA parameters suggesting that they are not influencing the normal hemostasis process, and have high biocompatibility compared to the prior polyP inhibitors.

### Example 15

### Lead MPI Agents

Preferred MPIs exhibit minimal deviation over a range of concentrations from the buffer control in the case of thrombin generated over time, while maximizing the polyphosphate inhibition effects. Each of the parameters that can be extracted from thrombin generation assays, as shown in FIG. 23, should lie within the standard deviation of the buffer control. A preferred MPI for in vivo use may be identified by excluding potential MPIs with undesirable deviation from this control. From the above data, it is noted that while most MPI candidates do not cause variance from the behavior observed for the buffer control at low concentrations (10-20 μg/mL), certain agents have large changes in thrombin generation behavior at high concentrations (most notably MPI 3 and MPI 9). However, specific MPI candidates demonstrate excellent biocompatibility (MPI 1, MPI 2, MPI 4, MPI 6, MPI 8), with enhanced safety over MPIs that resulted in large changes in coagulation. MPI 3, MPI 5, MPI 7, and MPI 9 were therefore not advanced due to their minor effects on thrombin generation in the TF triggered system. Three candidates stood out as strong agents which demonstrate no influence on blood clotting parameters in the absence of polyP while also demonstrating highly effective polyP inhibition activity at much lower concentrations compared to other MPI candidates: MPI 1, MPI 6 and MPI 8. These properties are extracted from the lower IC₅₀ values (Table 7) of these three candidates relative to both UHRA-8 and UHRA-10, as well as other MPI compounds.

Although favorable compatibility behavior is seen in MPI 2 and MPI 4, neither produced efficient high polyP inhibition. Furthermore, all the MPI compounds exhibit less impact on thrombin generation than that observed for UHRA-8 and UHRA-10, thereby highlighting the enhanced compatibility and minimal effect on hemostasis that this new family of polyphosphate inhibitors possesses.

Selected lead MPI agents were further characterized in depth to investigate their compatibility with more complex and sensitive systems. These measurements were performed to confirm that the MPI candidates do not exhibit incompatibility with blood components that might preclude them from therapeutic applications, an issue that is common to many conventional polycationic macromolecules. These studies also confirm that the new design of molecules resulted in significant improvements, which were enabled by their switchable protonation behavior and minimal charge under physiological conditions.

### Example 16

### Influence of MPIs on Human Platelets

Platelet activation was determined via expression of platelet activation marker CD62P in human platelet rich plasma (PRP) after incubation with MPIs. TRAP-6 treated platelets were used as a positive control and buffer added to PRP was a normal control. The degree of platelet activation was measured via flow cytometry with results shown in FIG. 24. The activation levels of platelets in the presence of lead MPI candidates were not significantly different compared to the buffer control. While many polycationic species exhibit unfavorable interactions with blood components due to non-specific interactions (e.g., polyamidoamine dendrimers) (Jones, ibid.) MPIs examined in the present assay did not induce significant platelet activation indicating improved biocompatibility.

### Example 17

### Influence of MPIs on Whole Blood Coagulation

Because the interaction of whole blood provides a closer representation of exposure to MPI polyP inhibitors, for example, during injection into the body the influence of MPIs on whole blood clotting were investigated. Blood components include many anionic biomolecules and blood cells that can potentially interact with these polycations via undesired non-specific pathways. Therefore, demonstrating compatibility with whole blood clotting is critical to establish the biocompatibility of these compounds. This information was obtained by measuring the whole blood clotting using rotational thromboelastometry (ROTEM). ROTEM measurements are considered to be closer to in vivo conditions than other coagulation assays because all blood components are present. (Bolliger, D. et al. Principles and Practice of Thromboelastography in Clinical Coagulation Management and Transfusion Practice. Transfus. Med. Rev. 2012, 26 (1), 1-13.) The clot time of whole blood, and strength of the final clot formed in presence of lead MPI compounds of the present invention, along with UHRA-8 and UHRA-10 were measured.

Buffer added to whole blood was used as a normal control. As shown in FIG. 25, MPIs did not alter the clot time or clot strength in comparison to the buffer control. However, UHRA-10 and UHRA-8 delayed the clot formation time (i.e., increased clot time) and also decreased clot strength as interpolated from the decreased maximum clot firmness. Although MPI 6 had slightly lower clot strength than MPI 1 or MPI 8, it was not significant compared to the control with buffer added to whole blood. Together, these data demonstrate that the lead MPI compounds of the present invention do not affect blood coagulation and may not interfere with hemostasis, a significant improvement from previous generation polyP inhibitors.

### Example 18

Influence of MPIs on fibrin clot morphology and fibril thickness To investigate the influence of MPIs of the present invention on final clot structure, clot structure was visualized using scanning electron microscopy. By comparing the fibrin clots formed in the presence and absence of MPIs, the influence of MPI on clot morphology and fibrin dimensions may be observed. As shown in FIG. 26, MPI 8 has minimal visible differences in the overall morphology and microstructure of the clot. Notably, there is no significant change in the thickness of the individual fibers formed in presence of MPI compared to the control. Thus, the MPI alone does not play a role in fibril thickening unlike other conventional polycationic polymers. The presence of charged polymers have previously been shown to increase the mass to length ratio of fibrils in fibrin clots, thickening the individual fibrils. (Carr, M. E. et al. Effect of Homo Poly(L-Amino Acids) on Fibrin Assembly: Role of Charge and Molecular Weight. Biochemistry 1989, 28 (3), 1384-1388.) Fibril thickening may cause instability in the final clot, resulting in increased susceptibility to clot lysis resulting in bleeding. (Shenkman, ibid.) However, certain polycations also increase thrombotic risk. (Jones, ibid.) Cationic polymers such as poly(L-arginine) or poly(L-lysine exhibit a concentration dependent increase in fibril thickness. (Carr, ibid.) The present results agree with the ROTEM data shown previously, confirming that the MPI compounds of the present invention are attractive therapeutic candidates.

In addition, we tested whether the addition of MPIs normalize clot structure formed in the presence of polyP. Fibrin clots formed in the presence of polyP, MPI 8 alone, MPI 8 together with polyP and buffer control were investigated using scanning electron microscopy (SEM). Micrographs obtained from SEM showing the clot microstructure and the calculated fibrin thickness are illustrated in FIG. 26. The overall clot morphology of the clot formed in the presence of MPI 8 is similar to the buffer control. PolyP added to fibrin clots shows a slight change in the density of the fibrils formed. However, this was normalized when MPI was also added (FIG. 26d). The average fiber diameters calculated from the micrographs are shown in FIG. 26e. The fibrin fibers formed in the presence of polyP have an increased thickness compared to buffer control consistent with previous reports. In the presence of only MPI 8, the clot fibers formed fibrils of a similar thickness to that of the buffer control. Thus, inhibition of polyP by MPI 8 normalized the clot microstructure.

### Example 19

### In Vivo Assessment of the Antithrombotic Activity of MPI Compounds—Mouse Cremaster Arteriole Thrombosis Model

Based on the in vitro studies detailed above, MPIs 1, 6, and 8 demonstrate strong binding behavior with minimal nonspecific interactions. MPIs 1, 6, and 8 also exhibited efficient reversal of both SC and LC polyP in human plasma. Investigations of the antithrombotic activities of MPI 1, 6, and 8, were performed in a mouse cremaster arteriole thrombosis model using intravital microscopy. MPIs were injected and the platelet accumulation and fibrin deposition were measured in comparison to the buffer control. Platelets and fibrin were then labelled with fluorescent antibodies to allow direct observation of the clot formation process. Clotting was then initiated via laser injury to the cremaster arteriole followed by observation of the clot formation over time, as shown in FIG. 27. Based on the results obtained from the average of 8 injuries, the rate of accumulation of platelets and fibrin measured from the fluorescence intensity, which is used as an indication of thrombus growth rate, showed a reduction in both platelet accumulation rate and total platelet accumulated for mice treated with 100 mg/kg MPI 1, MPI 6, and MPI 8 compared to mice that received only the saline control. Moreover, in mice treated with MPI 8, a reduction in the fibrin accumulation following the injuries was also observed, thereby underscoring antithrombotic action of these MPI candidates.

### Example 20

### In Vivo Assessment of the Antithrombotic Activity of MPI Compounds—Mouse Carotid Artery Thrombosis Model

The ability of MPI 8 to inhibit mouse carotid artery thrombosis in a FeCl₃ model was evaluated, wherein topical application of FeCl₃ was used to induce thrombosis (FIG. 28). UHRA-10 was used as a control. As shown in FIG. 28a, a dose of 100 mg/kg of MPI 8 was more effective in preventing occlusion, whereas a same dose of 100 mg/kg UHRA-10 did not prevent occlusion. MPI 8 at this concentration was also more effective at delaying the patency time as compared to a dose of 100 mg/kg UHRA-10, which displayed antithrombotic activity in this complex thrombosis model. Higher concentrations such as 200 mg/kg were tested, as shown in FIG. 28b, swith similar results for MPI 8 and UHRA-10. Because polyP is an accelerant in blood coagulation which does not affect normal hemostasis, it is possible that 100% patency will not be achieved by polyP inhibitors, unlike what one would expect with a higher concentration of UFH. (Travers, ibid.) As shown in FIG. 28c, a still higher dose of 300 mg/kg of MPI 8 and UHRA-10 were tested, further evidencing maximum patency in this model by these inhibitors. While the proportion of patentency achieved by MPI 8 (ca. 40% patent) remains the same from 200 mg/kg to 300 mg/kg, the efficacy of UHRA-10 decreases from 40% patent to 20% in this thrombosis model.

### Example 21

In vivo assessment of MPI safety—mouse tail bleeding

In view of the positive results over the broad range of in vitro and ex vivo measurements used to probe the biocompatibility of the MPI library, MPIs of the present invention were selected for further in vivo studies. To investigate the safety of the MPI candidates, mice were treated with high doses of MPI to confirm three key parameters: bleeding effect, acute toxicity, and long-term toxicity. The effect of the MPIs on bleeding was assessed in a mouse-tail bleeding model. In this experiment, mice were treated with the 3 MPIs, as well as saline and UFH as normal and positive controls, respectively. Following injection of MPI or controls, the bleeding time and hemoglobin loss were recorded after a tail clip to determine bleeding. As shown in FIG. 29, doses of MPIs of 100 mg/kg revealed minimal variance relative to the saline control. Conventional antithrombotic therapeutics often result in undesired bleeding side effects, even when administered at doses within the therapeutic window. When compared to the MPIs studied, UFH showed significantly increased bleeding time and a concomitant increase in hemoglobin loss. MPI 8 was examined at a dose much greater (300 mg/kg) than that at which it is effective as an antithrombotic in both the cremaster arteriole and the carotid artery models. Even at such high doses, MPI 8 showed no signs of bleeding compared to the saline control. This demonstrates a key benefit of these specifically designed polyP inhibitors, with the potential of developing antithrombotic therapeutics without bleeding issues.

### Example 21

In vivo assessment of MPI safety—acute toxicity Based on the antithrombotic activity of MPI compounds, MPI 8 was selected for a dose tolerance study. A single escalating dose tolerance study in mice was performed to determine the acute toxicity. Mice were injected with MPI 8 intravenously at high doses (250 mg/kg and 500 mg/kg) and sacrificed after 24 h. Mice injected with saline were used as a control. Determination of LDH, AST and ALT levels in serum was used as a measure of acute toxicity. As shown in FIG. 30, no significant changes were observed in comparison to control mice. LDH activity, a measure of necrotic or apoptotic cell damage, did not change significantly relative to the saline control. Aspartate aminotransferase (AST) levels in blood are commonly used as a marker for liver function. Alanine aminotransferase (ALT) is found primarily in liver and serum but occurs in other tissues as well. Hepatocellular injury often results in an increase of serum ALT levels and hence, these levels can be used as a marker for liver injury. MPI 8 did not elicit toxicity under the experimental conditions studied.

### Example 21

### In Vivo Assessment of MPI Safety—Chronic Toxicity

The long-term effects of exposure to MPI 8 in mice was quantified using an escalating dose injection study (100 to 500 mg/kg). Over the course of the 14-day study, mice body weights were monitored, as shown in FIG. 31. No significant difference between the test and control mice was observed. LDH activity in serum measured at the sacrifice also showed no significant difference relative to the controls. These results show that the reduced charge density and switchable protonation results in molecules with enhanced biocompatibility.

### Example 22

### Design and Synthesis of Macromolecular Heparin Antidotes

A schematic representation of MPIs as heparin reversal agents is shown in FIG. 32 Characteristics are described in Table 3.

### Example 23

### MPI Library Screening for Heparin Reversal Activity

Following structural characterization of the MPI library, heparin reversal activities of MPIs were determined in vitro using heparinized pooled normal plasma. As a method to determine optimal activity against LMWH (tinzaparin in this case) and UFH, the MPI library was screened using an activated partial thrombin time (aPTT) coagulation assay. A known heparin reversal agent, UHRA (Travers, ibid, Kalathottukaren and Abraham, ibid) was re-synthesized and used as a reference with demonstrated heparin reversal ability in plasma. To identify suitable MPIs for heparin reversal, the clot time was measured for each MPI at several concentrations as shown in FIG. 33a and FIG. 33b. A high concentration of UFH (4 U/mL) and LMWH (tinzaparin) at 1 U/mL were used for the initial neutralization studies. Two parameters were used for the initial selection process of a molecule which neutralized both heparins at low concentration, and without change in clotting time over a range of concentrations. As shown in FIG. 33c and FIG. 33d, these studies eliminated several MPI candidates that demonstrated reduced neutralization of UFH at a lower concentration, leaving MPIs 2, 3, 5, and 7 as compounds for further investigation. These compounds exhibited reversal of both tinzaparin and UFH at low concentrations, without change in clot times even at higher concentrations. As shown in FIGS. 33b and 33d, MPIs 1, 4, 6, and 8 displayed minimal to no reversal of UFH at 25 μg/mL in plasma with UFH, while exhibiting some reversal behavior at higher doses (50 μg/mL).

A preferred heparin antidote would demonstrate effective heparin reversal activity starting at a low concentration over a wide range of doses to ensure a broad therapeutic window, with activity unchanged over the range of concentrations examined. Of the compounds, MPIs 3, 5, and 7 demonstrated increased clotting time at higher concentrations of MPI, with significant deviances from the non-heparinized (buffer) control at a concentration of 150 μg/mL. MPI 2, on however, demonstrated complete heparin reversal and virtually unchanged clotting times in the dose range explored, from 10 to 50 μg/mL with tinzaparin (I U/mL), and 25 to 150 μg/mL with UFH (4 U/mL). In comparison to UHRA, this represents an increased activity for MPI 2, and the neutralization activity of MPI did not change over a range of concentrations studied. These results were further confirmed in a calibrated automated thrombography assay of all MPI compounds in heparinized (UFH, 0.5 U/mL) plasma by measuring thrombin generation (FIG. 34).

### Example 24

### MPI 2 Provides Potent Reversal Activity in Plasma Against UFH, LMWH and Fondaparinux

To further investigate the efficacy of MPI 2 as a universal heparin reversal agent, calibrated automated thrombography was used to evaluate the impact of MPI on thrombin generation in heparinized plasma. The amount of thrombin generated was assayed by measuring fluorescence intensity over time using a fluorogenic substrate calibrated to thrombin (FIG. 35a to 35_{c}). The effects of MPI 2 on thrombin generation were recorded at a series of concentrations from 0 (buffer control) to 10 μg/mL, against three different types of heparin: UFH, enoxaparin (LMWH), and fondaparinux (FIG. 35d to 35f). By measuring over these range of concentrations, it was possible to directly extract the half maximal inhibitory concentration (IC₅₀) of MPI 2 towards each of the heparins to evaluate its efficacy as a heparin reversal agent. In this concentration dependent study, it was found that MPI 2 exhibited an IC₅₀ of 1.0±0.6 μg/mL against 0.5 U/mL of UFH and 1.2±0.2 μg/mL against 0.3 U/mL of enoxaparin, a low molecular weight heparin. These values provide evidence of effective reversal of the naturally derived heparin therapeutics. In turn, MPI 2 demonstrated exceptional neutralization of fondaparinux, with an IC₅₀ of 3.0±2.1 μg/mL against 0.5 μg/mL fondaparinux.

Beyond demonstrating high heparin neutralization activity of MPI 2, the results from thrombin generation curves demonstrate that upon reversal of the heparin therapeutic, clotting properties in neutralized samples return to conditions analogous to those observed when no heparin was added to the plasma as in the buffer control. This is shown in FIG. 36, in which different doses of MPI 2 are plotted against lag time, total thrombin generation, time to peak thrombin generation, and maximum thrombin generation. This combination of factors characterizes clotting response in a system composed of plasma, heparin, and MPI 2. An ideal heparin reversal agent returns all of the above factors to the conditions observed in heparin free plasma, resulting in minimal disturbances in the clotting cascade when these compounds are applied in more complex biological systems. As shown in FIG. 36, MPI 2 nearly completely reverses the effect of all 3 heparin therapeutics including enoxaparin (LMWH) and fondaparinux against which it was measured, demonstrating favorable properties.

### Example 25

### MPI 2 Provides Minimal Clotting Disruption in Overdose Models in Plasma

To determine the range of concentrations that MPI is effective against UFH, the neutralization activity of MPI 2 was compared to both commercially available protamine sulfate (PS) and UHRA. The dose range studied was from 0-250 μg/mL. As shown in FIG. 37, the green shaded regions depict the effective range and overdose behavior shown in the yellow shaded regions. While PS exhibits heparin neutralization at lower doses, with clotting times returning to values close to the buffer control at optimum dose, PS exhibits clear anticoagulant behavior at higher doses. Similarly, UHRA shows excellent heparin neutralization over a range of doses studied, and slight anticoagulant behavior at higher doses. MPI 2 nearly completely neutralizes UFH in heparinized plasma at concentrations lower than UHRA. When higher doses were tested, there is almost no anticoagulant effect as typical of PS and UHRA.

The same assay was performed with plasma that was heparinized with a LMWH, tinzaparin. This experiment is critical because it highlights the significant increase in utility of MPI 2 over PS as a heparin antidote. PS has almost no neutralization activity on LMWH, shown in FIG. 37b. While both UHRA and MPI 2 demonstrated LMWH neutralization ability, there is a lack of change in clotting time when MPI 2 is used at high concentrations as is desired in a clinical therapeutic.

### Example 26

### MPI 2 Reverses UFH in Whole Blood

To determine whether the heparin reversal activity of MPI 2 persists in whole blood, rotational thromboelastometry (ROTEM) was performed. This viscoelastic method allows for direct investigation of the effect of anticoagulant alone (UFH) as well as the effect of added heparin antidotes (MPI 2 and UHRA are shown above, and FIG. 38) on hemostasis. In this experiment, fresh human whole blood was combined with combinations of UFH and heparin antidote, demonstrating that near complete return to buffer conditions was observed upon addition of only 20 μg/mL of MPI 2. When no heparin antidote is used, no noticeable clotting is observed in the observation window (5000s). Upon treatment with MPI 2, both the lag time and the profile of the thromboelastogram return a trace that is nearly identical to that observed in untreated whole blood (FIG. 38a). With UHRA there is no return to the clotting profile of the buffer control, even upon addition of 100 μg/mL. Although this dose exhibited heparin neutralization in aPTT, whole blood is composed of the complex mixture of all blood components including blood cells, platelets, proteins and other anionic macromolecules that have potential interactions with the MPI. The resulting ROTEM profile and clot time observed for UHRA in heparinized whole blood that is significantly different than that of the buffer control and of the positive UFH control (FIG. 5a and FIG. 38b) indicates that charges on UHRA may interact with specific blood components.

### Example 27

MPI 2 Compatibility with Blood Components and Whole Blood Clotting in the Absence of Heparin—Flow Cytometry

While heparin reversal efficacy is a key property of the MPI compounds of the present invention, a heparin antidote must further exhibit hemocompatibility to be used as a safe heparin reversal agent, and the antidote must not interfere with normal hemostasis, clot properties or clot severity whether bound to heparin or freely circulating. Accordingly, we determined whether the addition of MPIs of the present invention to whole blood have an impact on hemostasis. To demonstrate that even at extremely high doses, MPIs do not induce platelet activation, (activation of platelets is a frequent effect of highly positively charged macromolecules, such as polyethyleneimine (PEI) or polyamidoamine (PAMAM) dendrimers (Dubois, ibid, Hu, ibid)), platelet activation in platelet rich plasma (PRP) was studied using flow cytometry. When compared to positive and negative controls, thrombin receptor activating peptide 6 (TRAP) and buffer/platelet poor plasma respectively, addition of MPI 2 did not cause an increase in platelet activation, even at concentrations as high as 200 μg/mL, while the IC₅₀ in plasma of MPI 2 ranged from 1-5 μg/mL towards various heparins.

### Example 28

MPI 2 Compatibility with Blood Components and Whole Blood Clotting in the Absence of Heparin—ROTEM

To further demonstrate the compatibility of MPI 2 with blood components, blood coagulation in whole blood using ROTEM was performed (FIG. 39b-d) at 100 μg/mL, to model an overdose scenario of five times the 20 μg/mL dose of MPI 2 shown to be sufficient to completely reverse UFH in the same system (FIG. 39). MPI 2 demonstrated no significant change in the clot time when compared to the negative control (not significant, FIG. 6c), with a strong potential of compatibility with normal hemostasis. A second key parameter that can be extracted from ROTEM is maximum clot firmness (MCF), an indicator of the stability of the overall clot structure. As seen in FIG. 39d, the maximum clot firmness also remained unchanged when whole blood was clotted in the presence of buffer or with 100 μg/mL of MPI 2. However, a more pronounced effect on normal clotting were observed for 100 μg/mL dose of UHRA, exhibiting both a significantly increased clot time and a decrease in the maximum clot firmness of more than 20%. The increased clot time for UHRA implies that high doses of this heparin reversal agent interfere with normal hemostasis. The trend in both clot time and MCF were related to the quantity of charge presented on the polymer scaffold. More positive charge translated directly to a higher degree of interference with whole blood clotting.

### Example 29

### MPI 2 Reverses Both UFH and Enoxaparin—Mouse Bleeding Model

The efficacy of MPI 2 as a universal heparin reversal agent in vivo was determined using a mouse tail bleeding model. Mice were administered either 200 U/kg UFH or 200 U/kg enoxaparin followed by the antidote (MPI 2) or negative saline control. The bleeding time was recorded, and the hemoglobin loss was quantified. To examine the effect of MPI 2 as a UFH antidote, two MPI 2 doses were studied. When given 20 mg/kg of MPI 2, bleeding times and hemoglobin loss were both significantly decreased relative to UFH alone, however, when the dose was increased to 30 mg/kg, bleeding times and hemoglobin loss were decreased to levels similar to the saline control (FIGS. 40a and 40b). When the bleeding study was carried out on mice that were administered 200 U/kg enoxaparin, similar results were observed when heparin reversal was achieved with MPI 2. After addition of 20 mg/kg of MPI 2, a decrease in bleeding time and hemoglobin loss to levels similar to those in the saline control was observed (FIGS. 40c and 40d). These results indicate that the in vivo heparin reversal activity of MPI 2 parallels vitro studies. Moreover, MPI 2 demonstrates reversal of enoxaparin in the mouse bleeding model at similar doses to those used for UFH, demonstrating its efficacy against LMWHs.

### Example 30

MPI 2 does not Affect Bleeding in Mice in the Absence of Heparin

To determine the effect of MPI heparin antidotes of the present invention on bleeding in vivo, a mouse tail bleeding model was used in the absence of heparins. C57/BL6 mice were administered doses of MPI 2 that were five-fold (100 mg/kg) and ten-fold (200 mg/kg) the therapeutic doses demonstrated in the heparin reversal studies above. As illustrated in FIG. 41, no significant increases in bleeding time and hemoglobin loss were observed in mice dosed with either overdose quantity of MPI 2, with bleeding behavior for these mice closely matching the corresponding saline control.

### Example 31

### Inhibition of Nucleic Acids by MPI 3

To determine the activities of MPIs as nucleic acid inhibitors. PolyIC (Polyinosinic:polycytidylic acid) was used as a nucleic acid for this screening studies of MPI compounds of the present invention. A plate-based coagulation assay was used to compare candidate compounds Data is shown in FIG. 42. Among MPIs tested MPI-3 was found to inhibit polyIC induced blood coagulation in human plasma. We further investigated the inhibition of accelerated plasma clotting by low molecular Poly I:C at different concentrations. Results are shown in FIG. 43. Inhibition of contact pathway of blood coagulation is shown in FIG. 44. We used a substrate S2302 that may be cleaved by FXIIa as a measure of the inhibition activity by MPI 3. MPI 3 was shown to be effective in inhibiting FXIIa generation with cleavage of the substrate. Thrombin generation in presence of poly I:C and inhibition in presence of MPI 3 is shown in FIG. 45. These data show that MPI 3 inhibits thrombin generation. Divese parameters from CAT assay are shown. MPI 3 is also effective in correcting the anti-fibrinolytic effect of poly I:C (FIG. 46).

### Example 32

### Cecal Puncture and Ligation (Sepsis) Model

Thrombotic complications and cytokine storm are hallmarks of disease conditions such as sepsis. Organ damage associated with such complications is highly prevalent. Extracellular DNA-induced activation of blood coagulation and induction of inflammation may occur. Molecules which can prevent extracellular DNA-induced blood coagulation are hypothesized to prevent such complications. MPI 3 is directed against the polyanionic neutrophil extracellular traps (NETs) that neutrophils release in response to infection. To ensure inhibitor bioavailability throughout the study, 3 three doses of MPI 3 were administered every 2 hours after CLP surgery. Eight hours after surgery, mice were euthanized, and blood was collected for further analysis. FIG. 47 B shows an increase in extracellular DNA levels in the test mice in which sepsis was induced. Extracellular DNA levels were lower in mice that received 100 mg/kg MPI 3 following CLP compared to the control mice that received vehicle only. The levels of the TAT complex in the plasma collected at euthanasia were higher in the CLP group than in the SHAM procedure control group (FIG. 47A). TAT concentrations were lower in the CLP mice that received MPI 3 than those receiving vehicle, but the difference did not achieve statistical significance. Thrombin generation assays (FIGS. 47C and 47D) indicated that thrombin peak height was higher and time to peak was shorter in CLP mice compared to sham-operated controls. Administration of MPI 3 at 100 mg/kg was associated with a lower thrombin peak height. There was an increase in cytokines and chemokines in the plasma of the CLP mice as compared to the SHAM group, indicating an inflammatory response. The heatmap of the cytokine panel indicates that the cytokines/chemokine response was blunted in CLP mice receiving MPI 3 at 100 mg/kg (FIG. 48).

### Example 33

### Specificity of MPI 8 Toward Polyp in ADP-Mediated Platelet Activation

Studies were designed to evaluate the specificity of MPI 8 as a polyP inhibitor. Adenosine diphosphate (ADP) is a naturally occurring negatively charged phosphate containing molecule. We investigated whether polycationic MPI 8 binds to ADP (which is anionic) and alters its activity. For this purpose, we used an assay studying ADP-mediated platelet activation. FIG. 49 shows platelet activation when ADP and MPI 8 were mixed together. ADP (final [ADP]=40 μM) was mixed with human PRP (90 μL) for 15 minutes at 37° C., and then platelet activation was measured by measuring binding of anti-CD62P-PE antibody using flow cytometry (N=3 donors). There was no CD62P signal when MPI-8 at any concentration was incubated with PRP in the absence of ADP. These results show that the presence of MPI 8 does not alter the activity of ADP, indicating that MPI 8 does not inhibit ADP-mediated platelet activation. When PRP was pre-incubated with MPI 8 and then platelets were activated by the addition of ADP, there was no significant difference in CD62P signal between platelets activated with and without MPI 8 indicating that ADP is not interacting with MPI 8. (FIG. 50.)

### Example 34

### Saphenous Vein Hemostasis Model

The saphenous vein hemostasis model was used to assess hemostatic clot formation at the site of vascular injury following laser-induced rupture of the saphenous vein wall under intravital microscopy, with results shown in FIGS. 51 and 52. After the initial injury in vehicle-treated wild-type (WT) mice and drug treated WT mice, platelets immediately began adhering to the site of vascular injury to form a visible platelet-rich clot. Fibrin also began forming around the platelets at the site of vascular injury. The hemostatic response to vascular injury and clot stability increased after each subsequent injury and continued to grow. Platelet fibrin hemostatic clot formation in MPI 8 treated mice was similar to vehicle control with no detectable decrease in fibrin formation or platelet accumulation as analyzed by the dynamics of platelet mean fluorescent intensity (MFI) and fibrin MFI through repetitive injury. These findings indicate that administering MPI 8 intravenously to mice did not result in increased bleeding.

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

We claim:

1. A method of preventing and/or treating thrombosis, comprising administering a macromolecular polyphosphate inhibitor (MPI) to a subject wherein said administering prevents and/or treats said thrombosis.

2. The method of claim 1, wherein said MPI comprises one or more cationic binding groups (CBGs,) and one or more biocompatible scaffolds.

3. The method of claim 2, wherein said one or more CBGs is a linear alkyl amine.

4. The method of claim 2, wherein said one or more biocompatible scaffolds is a polyethylene glycol (PEG) scaffold and/or a polyglycerol scaffold.

5. The method of claim 4, wherein said MPI is MPI 8.

6. The method of claim 1, wherein said subject is a human subject.

7. The method of claim 1, wherein said administering is parenteral administering.

8. A method of reversing anticoagulation, comprising administering a macromolecular polyphosphate inhibitor (MPI) to a subject wherein said administering prevents and/or treats said anticoagulation.

9. The method of claim 8, wherein said anticoagulation is heparin anticoagulation, UFH heparin anticoagulation, enoxaparin anticoagulation, tinzaparin anticoagulation, dalteparin anticoagulation, and fondaparinux anticoagulation.

10. The method of claim 8, wherein said MPI comprises one or more cationic binding groups (CBGs,) and one or more biocompatible scaffolds.

12. The method of claim 10, wherein said one or more CBGs is a linear alkyl amine.

13. The method of claim 10, wherein said one or more biocompatible scaffolds is a polyethylene glycol (PEG) scaffold and/or a polyglycerol scaffold.

14. The method of claim 13, wherein said MPI is MPI 2.

15. The method of claim 8, wherein said subject is a human subject.

16. The method of claim 8, wherein said administering is parenteral administering.

17. A composition comprising:
a) one or more cationic binding groups (CBGs);
b) one or more biocompatible scaffolds; and
c) a pharmaceutically acceptable carrier.

18. The composition of claim 17, wherein said one or more CBGs is a linear alkyl amine.

19. The composition of claim 17, wherein said one or more biocompatible scaffolds is a polyethylene glycol (PEG) scaffold and/or a polyglycerol scaffold.

20. The composition of claim 17, wherein said composition is MPI 8.

21. The composition of claim 17, wherein said composition is MPI 2.

22. Use of a composition of any of claims 17-21.

23. Use of a composition of any of claims 17-21 for the treatment of disease in a subject.

24. A composition comprising a polymeric compound, comprising:
a) a hyperbranched polyglyercol core;
b) a plurality of polyethylene glycol (PEG) chains covalently attached to said hyperbranched polyglyercol core; and
c) a plurality of linear alkylamine moieties covalently attached to said hyperbranched polyglyercol core.

25. The composition of claim 1, wherein said plurality of linear alkylamine moieties have structures of formula (I): 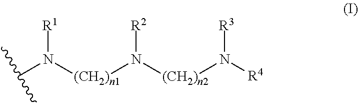
or a pharmaceutically salt thereof, wherein:
a) n1 and n2 are independently selected from 2 and 3;
b) R¹, R², R³, and R⁴ are independently selected from C₁-C₃ alkyl; and
c) 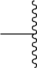
is a point of attachment to said hyperbranched polyglyercol core.

26. The composition of claim 25, wherein n1 and n2 are 2.

27. The composition of claim 25, wherein n1 and n2 are 3.

28. The composition of claim 25, wherein R¹, R², R³, and R⁴ are methyl.

29. The composition of claim 24, wherein said linear alkylamines have a structure selected from: 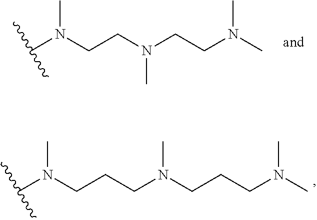 [SMILES: *N(C)CCN(C)CCN(C)C ; *N(C)CCCN(C)CCCN(C)C]
or a salt thereof.

30. The composition of claim 24, wherein said polymeric compound has a number average molecular weight of about 8 kDa to about 25 kDa, or about 10 kDa to about 23 kDa. In some embodiments, the core has a molecular weight of about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, or about 25 kDa, or any range therebetween.

31. The composition of claim 24, wherein said polymeric compound has an average of about 10 to about 25 linear alkylamine moieties covalently attached to said hyperbranched polyglyercol core.

32. The composition of claim 31, wherein said polymeric compound has an average of about 10, about 11, about 12, about 13, about 14, about 15, about 16, about 17, about 18, about 19, about 20, about 21, about 22, about 23, about 24, or about 25 linear alkylamine moieties covalently attached to said core.
